(19) United States
(12) Patent Application Publication
(10) Pub. No.: US 2022/0162643 A1
(43) Pub. Date: May 26, 2022

(54) OPTIMIZED PHENYLANANINE HYDROXYLASE EXPRESSION

(71) Applicant: American Gene Technologies International Inc., Rockville, MD (US)

(72) Inventors: Tyler Lahusen, Rockville, MD (US); Lingzhi Xiao, Rockville, MD (US); Charles David Pauza, Rockville, MD (US)

(73) Assignee: American Gene Technologies International Inc., Rockville, MD (US)

(21) Appl. No.: 17/610,111

(22) PCT Filed: Jun. 1, 2020
(86) PCT No.: PCT/US2020/035584

§ 371 Date: Nov. 9, 2021

Related U.S. Application Data
(60) Provisional application No. 62/855,506, filed on May 31, 2019.

(51) Int. Cl. C12N 15/86; C12N 9/02

(52) U.S. Cl. CPC C12N 15/86; C12N 9/0071; A61K 48/00; C12N 2740/15043; C12N 2740/15023; C12Y 114/16001

(57) ABSTRACT

A lentiviral vector system for expressing a lentiviral particle is disclosed. The lentiviral vector system includes a therapeutic vector. The lentiviral vector system produces a lentiviral particle that encodes a codon-optimized PAH for upregulating PAH expression in the cells of a subject afflicted with phenylketonuria (PKU).

### PRIORITY AND INCORPORATION BY REFERENCE

This application claims priority to U.S. Provisional Application No. 62/855,506 entitled Codon-Optimized Phenylalanine Hydroxylase, filed May 31, 2019, which is incorporated by reference in its entirety.

### FIELD

Aspects of the disclosure relate to genetic medicines for treating phenylketonuria (PKU). More specifically, aspects of the disclosure relate to lentiviral vectors, including codon-optimized PAH-containing lentiviral vectors.

### BACKGROUND

Phenylketonuria (PKU) refers to a heterogeneous group of disorders that can lead to intellectual disability, seizures, behavioral problems, and impaired growth and development in affected children if left untreated. The mechanisms by which hyperphenylalaninemia results in intellectual impairment reflect the surprising toxicity of high dose phenylalanine and involve hypomyelination or demyelination of nervous system tissues. PKU has an average reported incidence rate of 1 in 12,000 in North America, affecting males and females equally. The disorder is most common in people of European or Native American ancestry and reaches much higher levels in the eastern Mediterranean region.

Neurological changes in patients with PKU have been demonstrated within one month of birth, and magnetic resonance imaging (MRI) in adult PKU patients has shown white matter lesions in the brain. The size and number of these lesions relate to blood phenylalanine concentrations. The cognitive profile of adolescents and adults with PKU compared with control subjects can include significantly reduced IQ, processing speed, motor control and inhibitory abilities, and reduced performance on tests of attention.

The majority of PKU is caused by a deficiency of hepatic phenylalanine hydroxylase (PAH). PAH is a multimeric hepatic enzyme that catalyzes the hydroxylation of phenylalanine (Phe) to tyrosine (Tyr) in the presence of molecular oxygen and catalytic amounts of tetrahydrobiopterin (BH₄), its nonprotein cofactor. In the absence of sufficient expression of PAH, phenylalanine levels in the blood increase leading to hyperphenylalaninemia and harmful side effects in PKU patients. Decreased or absent PAH activity can lead to a deficiency of tyrosine and its downstream products, including melanin, 1-thyroxine and the catecholamine neurotransmitters including dopamine.

PKU can be caused by mutations in PAH and/or a defect in the synthesis or regeneration of PAH cofactors (i.e., BH₄). Notably, several PAH mutations have been shown to affect protein folding in the endoplasmic reticulum resulting in accelerated degradation and/or aggregation due to missense mutations (63%) and small deletions (13%) in protein structure that attenuate or largely abolish enzyme catalytic activity.

In general, three major phenotypic groups are used to classify PKU based on blood plasma Phe levels, dietary tolerance to Phe and potential responsiveness to therapy. These groups include classical PKU (Phe >1200 μM), atypical or mild PKU (Phe is 600-1200 μM), and permanent mild hyperphenylalaninemia (HPA, Phe 120-600 μM).

Detection of PKU relies on universal newborn screening (NBS). A drop of blood collected from a heel stick is tested for phenylalanine levels in a screen that is mandatory in all 50 states of the USA.

Currently, lifelong dietary restriction of Phe and BH₄ supplementation are the only two available treatment options for PKU, where early therapeutic intervention is critical to ensure optimal clinical outcomes in affected infants. However, costly medication and special low-protein foods impose a major burden on patients that can lead to malnutrition, psychosocial or neurocognitive complications notably when these products are not fully covered by private health insurance. Moreover, BH₄ therapy is primarily effective for treatment of mild hyperphenylalaninemia as related to defects in BH₄ biosynthesis, whereas only 20-30% of patients with mild or classical PKU are responsive. Thus, there is need for new treatment modalities for PKU as an alternative to burdensome Phe-restriction diets.

Genetic medicines have the potential to effectively treat PKU. Genetic medicines may involve delivery and expression of genetic constructs for the purposes of disease therapy or prevention. Expression of genetic constructs may be modulated by various promoters, enhancers, and/or combinations thereof.

### SUMMARY

In an aspect, a viral vector is provided comprising a therapeutic cargo portion, wherein the therapeutic cargo portion comprises a modified PAH sequence or variant thereof, for modulated phenylalanine hydroxylase (PAH) expression. In further aspects, a viral vector is provided comprising a therapeutic cargo portion, wherein the therapeutic cargo portion comprises a codon-optimized PAH sequence or variant thereof, for enhanced PAH expression, and optionally a promoter and a liver-specific enhancer, wherein the PAH sequence or variant thereof is operatively controlled by both the promoter and the liver-specific enhancer.

In embodiments, the viral vector comprises a codon-optimized PAH sequence or variant thereof having at least 75 percent, at least 80 percent, at least 85 percent, at least 90 percent, at least 95 percent sequence identity with SEQ ID NO: 70. In embodiments, the viral vector comprises a codon-optimized PAH sequence or variant thereof comprising the sequence of SEQ ID NO: 70.

In an aspect, a viral vector is provided comprising a codon-optimized PAH sequence or variant thereof, wherein the codon-optimized PAH sequence or variant thereof having at least 75 percent, at least 80 percent, at least 85 percent, at least 90 percent, or at least 95 percent sequence identity to SEQ ID NO: 71. In embodiments, the codon-optimized PAH sequence or variant thereof comprises the sequence of SEQ ID NO: 71. In embodiments, the viral vector further comprises a therapeutic cargo portion that comprises the codon-optimized PAH sequence or variant thereof, a promoter, and a liver-specific enhancer, wherein the codon-optimized PAH sequence or variant thereof is operatively controlled by both the promoter and the liver-specific enhancer.

In an aspect, a viral vector is provided comprising a codon-optimized PAH sequence or variant thereof, wherein the codon-optimized PAH sequence or variant thereof having at least 75 percent, at least 80 percent, at least 85 percent, at least 90 percent, or at least 95 percent sequence identity to SEQ ID NO: 72. In embodiments, the codon-optimized PAH sequence or variant thereof comprises the sequence of SEQ ID NO: 72. In embodiments, the viral vector further comprises a therapeutic cargo portion that comprises the codon-optimized PAH sequence or variant thereof, a promoter, and a liver-specific enhancer, wherein the codon-optimized PAH sequence or variant thereof is operatively controlled by both the promoter and the liver-specific enhancer.

In embodiments, the liver-specific enhancer comprises a prothrombin enhancer. In embodiments the prothrombin enhancer comprises a sequence having at least 75 percent, at least 80 percent, at least 85 percent, at least 90 percent, or at least 95 percent identity with SEQ ID NO: 3. In embodiments, the prothrombin enhancer comprises the sequence of SEQ ID NO: 3.

In embodiments, the promoter comprises a liver-specific promoter. In embodiments, the liver-specific promoter comprises a hAAT promoter. In embodiments, the hAAT promoter comprises a sequence having at least 75 percent, at least 80 percent, at least 85 percent, at least 90 percent, or at least 95 percent identity with SEQ ID NO: 4. In embodiments, the hAAT promoter comprises the sequence of SEQ ID NO: 4.

In embodiments, the therapeutic cargo portion further comprises a beta globin intron. In embodiments, the beta globin intron comprises a sequence having at least 75 percent, at least 80 percent, at least 85 percent, at least 90 percent, or at least 95 percent identity with SEQ ID NOS: 5 or 6. In embodiments, the beta globin intron comprises the sequence of SEQ ID NOS: 5 or 6.

In embodiments, the therapeutic cargo portion further comprises at least one hepatocyte nuclear factor binding site. In embodiments, the hepatocyte nuclear factor binding site comprises a sequence having at least 75 percent, at least 80 percent, at least 85 percent, at least 90 percent, or at least 95 percent identity with SEQ ID NOS: 7 (1XHNF1), 8 (5XHNF1), 9 (1XHNF1/4), or 10 (3XHNF1/4). In embodiments, the hepatocyte nuclear factor binding site comprises the sequence of SEQ ID NOS: 7, 8, 9, or 10.

In embodiments, the at least one hepatocyte nuclear factor binding site is disposed downstream of the prothrombin enhancer.

In embodiments, the therapeutic cargo portion further comprises at least one small RNA sequence. In embodiments, the at least one small RNA sequence comprises a sequence having at least 75 percent, at least 80 percent, at least 85 percent, at least 90 percent, or at least 95 percent identity with SEQ ID NOS: 11 or 12. In embodiments, the at least one small RNA sequence is under the control of a first promoter and the PAH sequence is under the control of a second promoter. In embodiments, the first promoter is a H1 promoter. In embodiments, the second promoter is a liver-specific promoter.

In embodiments, the viral vector is a lentiviral vector or an adeno-associated viral vector. In embodiments, the viral vector is a lentiviral vector or another viral vector or non-viral system suitable for delivering the codon-optimized PAH sequence described herein. In embodiments, the viral vector is a lentiviral vector.

In an aspect, a viral vector is provided comprising a therapeutic cargo portion, wherein the therapeutic cargo portion comprises a codon-optimized PAH sequence or variant thereof comprising a sequence that shares greater than 95 percent sequence identity to SEQ ID NO: 70. In embodiments, the codon-optimized PAH sequence or variant thereof comprises SEQ ID NO: 70.

In an aspect, a viral vector is provided comprising a therapeutic cargo portion, wherein the therapeutic cargo portion comprises a codon-optimized PAH sequence or variant thereof comprising a sequence having at least 75 percent, at least 80 percent, at least 85 percent, at least 90 percent, or at least 95 sequence identity to SEQ ID NO 71. In embodiments, the codon-optimized PAH sequence or variant thereof comprises SEQ ID NO: 71.

In an aspect, a viral vector is provided comprising a therapeutic cargo portion: wherein the therapeutic cargo portion comprises a codon-optimized PAH sequence or variant thereof comprising a sequence having at least 75 percent, at least 80 percent, at least 85 percent, at least 90 percent, or at least 95 sequence identity to SEQ ID NO: 72. In embodiments, the codon-optimized sequence or variant thereof comprises SEQ ID NO: 72.

In an aspect, a viral vector is provided comprising a therapeutic cargo portion: wherein the therapeutic cargo portion comprises a codon-optimized PAH sequence or variant thereof comprising a sequence having at least 75 percent, at least 80 percent, at least 85 percent, at least 90 percent, or at least 95 percent sequence identity to SEQ ID NO: 73. In embodiments, the codon-optimized PAH sequence or variant thereof comprises the sequence of SEQ ID NO: 73.

In an aspect, a viral vector is provided comprising a therapeutic cargo portion: wherein the therapeutic cargo portion comprises a codon-optimized PAH sequence or variant thereof comprising a sequence having at least 75 percent, at least 80 percent, at least 85 percent, at least 90 percent, or at least 95 percent sequence identity to SEQ ID NO: 74. In embodiments, the codon-optimized PAH sequence or variant thereof comprises the sequence of SEQ ID NO: 74.

In an aspect, a viral vector is provided comprising a therapeutic cargo portion, wherein the therapeutic cargo portion comprises a codon-optimized PAH sequence or variant thereof comprising a sequence having at least 75 percent, at least 80 percent, at least 85 percent, at least 90 percent, or at least 95 percent sequence identity to SEQ ID NO: 75. In embodiments, the codon-optimized PAH sequence or variant thereof comprises the sequence of SEQ ID NO: 75.

In an aspect, a viral vector is provided comprising a therapeutic cargo portion: wherein the therapeutic cargo portion comprises a codon-optimized PAH sequence or variant thereof comprising a sequence having at least 75 percent, at least 80 percent, at least 85 percent, at least 90 percent, or at least 95 percent sequence identity to SEQ ID NO: 76. In embodiments, the codon-optimized PAH sequence or variant thereof comprises the sequence of SEQ ID NO: 76.

In an aspect, a viral vector is provided comprising a codon-optimized PAH sequence or variant thereof comprising a sequence having at least 75 percent, at least 80 percent, at least 85 percent, at least 90 percent, or at least 95 percent sequence identity to SEQ ID NO: 73. In embodiments, the codon-optimized PAH sequence or variant thereof comprises the sequence of SEQ ID NO: 73. In embodiments, the viral vector further comprises a therapeutic cargo portion that comprises a codon-optimized PAH sequence or variant thereof, a promoter, and a liver-specific enhancer, wherein the codon-optimized PAH sequence or variant thereof is operatively controlled by both the promoter and the liver-specific enhancer.

In an aspect, a viral vector is provided comprising a codon-optimized PAH sequence or variant thereof comprising a sequence having at least 75 percent, at least 80 percent, at least 85 percent, at least 90 percent, or at least 95 percent sequence identity to SEQ ID NO: 74. In embodiments, the codon-optimized PAH sequence or variant thereof comprises the sequence of SEQ ID NO: 74. In embodiments, the viral vector further comprises a therapeutic cargo portion that comprises the codon-optimized PAH sequence or variant thereof, and further comprises a promoter, and a liver-specific enhancer, wherein the codon-optimized PAH sequence or variant thereof is operatively controlled by both the promoter and the liver-specific enhancer.

In an aspect, a viral vector is provided comprising a codon-optimized PAH sequence or variant thereof comprising a sequence having at least 75 percent, at least 80 percent, at least 85 percent, at least 90 percent, or at least 95 percent sequence identity to SEQ ID NO: 75. In embodiments, the codon-optimized sequence or variant thereof comprises SEQ ID NO: 75. In embodiments, the viral vector further comprises a therapeutic cargo portion that comprises the codon-optimized PAH sequence or variant thereof, and further comprises a promoter, and a liver-specific enhancer, wherein the codon-optimized PAH sequence or variant thereof is operatively controlled by both the promoter and the liver-specific enhancer.

In an aspect, a viral vector is provided comprising a codon-optimized PAH sequence or variant thereof comprising a sequence having at least 75 percent, at least 80 percent, at least 85 percent, at least 90 percent, or at least 95 percent sequence identity to SEQ ID NO: 76. In embodiments, the codon-optimized sequence or variant thereof comprises SEQ ID NO: 76. In embodiments, the viral vector further comprises a therapeutic cargo portion that comprises the codon-optimized PAH sequence or variant thereof, and further comprises a promoter, and a liver-specific enhancer, wherein the codon-optimized PAH sequence or variant thereof is operatively controlled by both the promoter and the liver-specific enhancer.

In an aspect, a lentiviral particle produced by a packaging cell and capable of infecting a target cell is disclosed. In embodiments, the lentiviral particle comprises an envelope protein capable of infecting a target cell, and a viral vector as detailed herein.

In an aspect, a method of treating phenylketonuria (PKU) in a subject is disclosed. The method involves administering to the subject a therapeutically effective amount of a lentiviral particle as detailed herein.

In an aspect, use of a codon-optimized PAH sequence or variant thereof for treating PKU in a subject is provided. In another aspect, use of a codon-optimized PAH sequence or variant thereof to formulate a medicament for treating PKU in a subject is provided.

In an aspect, a codon-optimized PAH sequence or variant thereof for use in treating PKU in a subject is provided. In another aspect, a codon-optimized PAH sequence or variant thereof to formulate a medicament for use in treating PKU in a subject is provided.

### BRIEF DESCRIPTION OF THE DRAWINGS

FIG. 1 depicts an exemplary 3-vector lentiviral vector system in a circularized form.

FIG. 2 depicts an exemplary 4-vector lentiviral vector system in a circularized form.

FIG. 3 depicts linear maps of four exemplary lentiviral vectors containing variations of the prothrombin enhancer and hAAT promoter to regulate the expression of PAH.

FIGS. 4A-4B depict immunoblot data comparing levels of PAH in Hepa1-6 cells after transduction of hPAH and various forms of codon-optimized PAH sequences. FIG. 4A compares hPAH with the OPT2 codon-optimized PAH. FIG. 4B compares hPAH with the OPT3, OPT2/3, and OPT3/2 versions of codon-optimized PAH.

FIG. 5 depicts PAH RNA expression in Hepa1-6 cells transduced with lentiviral vectors expression hPAH and codon-optimized versions of PAH.

FIGS. 6A-6B depict immunoblot data comparing levels of codon-optimized PAH with HNF1 and HNF1/4 binding sites upstream of the prothrombin enhancer. FIG. 6A depicts immunoblot data in Hepa1-6 cells. FIG. 6B depicts immunoblot data in Hep3B cells.

FIG. 7 depicts immunoblot data comparing levels of codon-optimized PAH with a regulatory sequence containing either prothrombin enhancer/hAAT promoter/Minute Virus of Mouse intron or hAAT enhancer/transthyretin promoter/Minute Virus of Mouse intron.

FIG. 8 depicts immunoblot data comparing levels of codon-optimized PAH with a regulatory sequence containing a mutant WPRE sequence or short WPRE (WPREs) sequence, or a PAH or albumin 3′ UTR sequence.

### DETAILED DESCRIPTION

### Overview of the Disclosure

This disclosure relates to therapeutic vectors and delivery of the same to cells. In an aspect, the therapeutic vector is a viral vector comprising a therapeutic cargo portion: wherein the therapeutic cargo portion comprises: a codon-optimized PAH sequence or variant thereof; a promoter; and a liver-specific enhancer, wherein the PAH sequence or variant thereof is operatively controlled by both the promoter and the liver-specific enhancer. In embodiments, the vectors include codon-optimized PAH sequences or variants thereof, and/or a liver-specific enhancer. In embodiments, the vectors include a small RNA that regulates host (i.e., endogenous) PAH protein expression. In embodiments, the viral vector is a lentiviral vector.

### Definitions

Unless otherwise defined herein, scientific and technical terms used in connection with this disclosure shall have the meanings that are commonly understood by those of ordinary skill in the art. Further, unless otherwise required by context, singular terms shall include pluralities and plural terms shall include the singular. Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the disclosure are generally performed according to conventional methods well-known in the art and as described in various general and more specific references that are cited and discussed throughout the specification unless otherwise indicated. See, e.g.: Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual; Cold Spring Harbor Laboratory Press, Cold Spring Harbor, N.Y. (1998); and Coligan et al., Short Protocols in Protein Science, Wiley, John & Sons, Inc. (2003). Any enzymatic reactions or purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art.

As used herein, the singular forms “a”, “an” and “the” are used interchangeably and intended to include the plural forms as well and fall within each meaning, unless the context indicates otherwise. Also, as used herein, “and/or” refers to and encompasses any and all possible combinations of one or more of the listed items, as well as the lack of combinations when interpreted in the alternative (“or”).

All numerical designations, e.g., percent, pH, temperature, time, concentration, and molecular weight, including ranges, are approximations which can include variation, for example (+) or (−) an increment of 0.1% or 0.1. It is to be understood, although not always explicitly stated that all numerical designations are preceded by the term “about”. It also is to be understood, although not always explicitly stated, that the reagents described herein are merely exemplary and that equivalents of such are known in the art.

As used herein, the term “about” will be understood by persons of ordinary skill in the art and will vary to some extent depending upon the context in which it is used. If there are uses of the term which are not clear to persons of ordinary skill in the art given the context in which it is used, “about” will include the value and up to plus or minus 10% of the value. The term “about” also includes the exact value “X” in addition to minor increments of “X” such as “X”+0.1% or X−0.1%.

As used herein, the term “administration of” or “administering” means providing any of the disclosed vectors, vector compositions, pharmaceutical compositions, or other active agents disclosed herein to a subject in need of treatment in a form that can be introduced into that individual's body in a therapeutically useful form and therapeutically effective amount. Methods of administering the disclosed vectors, vector compositions, or other active agents can be any of the methods disclosed herein.

As used herein, the phrase “coding sequence” describes any viral vector sequence capable of being transcribed or reverse transcribed. A “coding sequence” includes, without limitation, exogenous sequences (e.g., sequences on vectors that have been transduced or transfected into cells) capable of being transcribed or reverse transcribed.

As used herein, the term “codon-optimized” means modulating a coding sequence according to at least one of the following; (i) substituting naturally occurring codon sequences with alternative codons that preserve the amino acid sequence of the encoded protein but alter the composition and/or structure of the encoding RNA; (ii) modulating the guanosine cytosine content of the coding sequence relative to the naturally occurring guanosine cytosine content of the coding sequence; (iii) modulating the number of CpG sites of the coding sequence relative to the number of CpG sites in naturally occurring coding sequence; and (iv) substituting the naturally occurring codon sequences with alternative codons relative to (ii) the guanosine cytosine content and/or (iii) the number of CpG sites. Codon optimization may comprise adjustment of codons in the context of tRNA expression in specific tissues and/or may comprise methods for evading the action of natural, tissue-specific shRNA or miRNA.

As used herein, the term “comprising” means that the compositions and methods include the recited elements, but not excluding others. “Consisting essentially of” when used to define compositions and methods, means excluding other elements of any essential significance to the composition or method. “Consisting of” means excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, the term “CpG site,” refers to regions of DNA where a cytosine nucleotide is followed by a guanine nucleotide in the linear sequence of bases along its 5′-3′ direction. CpG sites occur with high frequency in genomic regions called CpG islands (or CG islands). Cytosines in CpG dinucleotides can be methylated to form 5-methylcytosines. In mammals, 70% to 80% of CpG cytosines are methylated. Methylating the cytosine within a gene can change its expression.

As used here, the term “UTR” refers generally to an untranslated region of messenger RNA (mRNA) that remains after RNA splicing is completed. As used herein, “3′ UTR” refers to an untranslated region of mRNA that immediately follows the translation termination codon. The 3′UTR is not translated into a resulting protein.

As used herein, the term “adeno-associated viral vector,” refers to a synthetic delivery system which makes use of structural components of adeno-associated virus to deliver therapeutic DNA cargo into cells or tissues. The term “adeno-associated viral vector” may also be referred to herein as an “AAV vector”.

As used herein, the term “adeno-associated virus,” refers to a small virus that generates a mild immune response, is capable of depositing an extrachromosomal DNA copy of itself in a host cell, occasionally integrates a DNA copy into the host genome, and is relatively non-pathogenic. Adeno-associated virus includes numerous natural and synthetic serotypes, including but not limited to AAV2, as described herein.

As used herein, the term “AAV/DJ” (also referred to herein as “AAV-DJ”) is a serotype of an AAV vector engineered from different AAV serotypes, which mediates higher transduction and infectivity rates than wild type AAV serotypes.

As used herein, the term “AAV2” (also referred to herein as “AAV/2” or “AAV-2”) is a naturally occurring AAV serotype.

As used herein, the term “ApoE enhancer” refers to an Apolipoprotein E enhancer.

As used herein, the term “expression”, “expressed”, or “encodes” refers to the process by which polynucleotides are transcribed into mRNA or reverse transcribed into DNA and/or the process by which transcribed mRNA is subsequently translated into peptides, polypeptides, or proteins. Expression may include splicing of the mRNA in a eukaryotic cell or other forms of post-transcriptional modification or post-translational modification.

As used herein, the term “genetic medicine” or “genetic medicines” refers generally to therapeutics and therapeutic strategies that focus on genetic targets to treat a clinical disease or manifestation. The term “genetic medicine” encompasses gene therapy and the like.

As used herein, the term “hAAT” refers to a hAAT promoter.

As used herein, the term “hepatocyte nuclear factors” refers to transcription factors that are predominantly expressed in the liver. Types of hepatocyte nuclear factors include, but are not limited to, hepatocyte nuclear factor 1, hepatocyte nuclear factor 2, hepatocyte nuclear factor 3, and hepatocyte nuclear factor 4.

As used herein, the term “HNF” refers to hepatocyte nuclear factor. Accordingly, HNF1 refers to hepatocyte nuclear factor 1, HNF2 refers to hepatocyte nuclear factor 2, HNF3 refers to hepatocyte nuclear factor 3, and HNF4 refers to hepatocyte nuclear factor 4.

As used herein, the term “HNF binding site,” refers to a region of DNA to which an HNF transcription factor can bind. Accordingly, a HNF1 binding site is a region of DNA to which HNF1 can bind, and a HNF4 binding site is a region of DNA to which HNF4 can bind.

As used herein, the term “human beta globin intron” refers to a nucleic acid segment within the human beta globin gene that is spliced out during RNA maturation, and does not code for a protein.

As used herein, the terms “individual,” “subject,” and “patient” are used interchangeably herein, and refer to any individual mammal subject, e.g., murine, porcine, bovine, canine, feline, equine, nonhuman primate or human primate.

As used herein, the term “LV” refers generally to “lentivirus.” As a non-limiting example, reference to “LV-PAH” is reference to a lentivirus that contains a PAH sequence and expresses PAH. The PAH sequence may be a hPAH sequence or a codon-optimized PAH sequence.

As used herein, the term “LV-Pro-hAAT-PAH” refers to an LV vector comprising a prothrombin enhancer, a hAAT promoter, and a PAH sequence.

As used herein, the term “packaging cell line” refers to any cell line that can be used to express a lentiviral particle.

As used herein, the term “percent identity” or “percent sequence identity”, in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the “percent identity” or “percent sequence identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

As used herein, the term “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, the term “phenylalanine hydroxylase” may also be referred to herein as PA. The term phenylalanine hydroxylase includes nucleotide and peptide sequences of all wild type, variant, and codon-optimized PAH sequences, including fragments of PAH sequences. Without limitation, the term phenylalanine hydroxylase includes reference to SEQ ID NOS: 1, 2, and 70-76, and further includes variants having at least about 75% identity therewith.

As used herein, the term “hPAH” refers to a PAH sequence derived from a human or a human source, the codons of which have not been synthetically altered.

As used herein, the term “phenylketonuria”, which is also referred to herein as “PKU”, refers to the chronic deficiency of phenylalanine hydroxylase, as well as all symptoms related thereto including mild and classical forms of disease. Treatment of “phenylketonuria”, therefore, may relate to treatment for all or some of the symptoms associated with PKU.

As used herein, the term “prothrombin enhancer” is a region on the prothrombin gene that can be bound by proteins, which results in transcription of the prothrombin gene.

As used herein, the term “Pro” refers to a prothrombin enhancer.

As used herein, the term “rabbit beta globin intron” refers to a nucleic acid segment within the rabbit beta globin gene that is spliced out during RNA maturation, and does not code for a protein.

As used herein, the term “small RNA” refers to non-coding RNA that are generally about 200 nucleotides or less in length and possess a silencing or interference function. In other embodiments, the small RNA is about 175 nucleotides or less, about 150 nucleotides or less, about 125 nucleotides or less, about 100 nucleotides or less, or about 75 nucleotides or less in length. Such RNAs include microRNA (miRNA), small interfering RNA (siRNA), double stranded RNA (dsRNA), and short hairpin RNA (shRNA), small nuclear RNA (snRNA), and small nucleolar RNA (snoRNA). “Small RNA” of the disclosure should be capable of inhibiting or knocking-down gene expression of a target gene, generally through pathways that result in the degradation of the target gene mRNA or pathways that prevent translation of the target gene mRNA.

As used herein, the term “shPAH” refers to a small hairpin RNA that targets PAH.

As used herein, the term “SEQ ID NO” is synonymous with the term “Sequence ID No.”

As used herein, the term “thyroxin binding globulin,” is a transport protein responsible for carrying thyroid hormones in the bloodstream. As used herein, the abbreviation “TBG” refers to thyroxin binding globulin.

As used herein, the term “therapeutically effective amount” refers to a sufficient quantity of the active agents of the present disclosure, in a suitable composition, and in a suitable dosage form to treat or prevent the symptoms, progression, or onset of the complications seen in patients suffering from a given ailment, injury, disease, or condition. The therapeutically effective amount will vary depending on the state of the patient's condition or its severity, and the age, weight, etc., of the subject to be treated. A therapeutically effective amount can vary, depending on any of a number of factors, including, e.g., the route of administration, the condition of the subject, as well as other factors understood by those in the art.

As used herein, the term “therapeutic vector” includes, without limitation, reference to a lentiviral vector or an adeno-associated viral (AAV) vector. Additionally, as used herein with reference to the lentiviral vector system, the term “vector” is synonymous with the term “plasmid”. For example, the 3-vector and 4-vector systems, which include the 2-vector and 3-vector packaging systems, can also be referred to as 3-plasmid and 4-plasmid systems.

As used herein, the term “treatment” or “treating” generally refers to an intervention in an attempt to alter the natural course of the subject being treated, and can be performed either for prophylaxis or during the course of clinical pathology. Desirable effects include, but are not limited to, preventing occurrence or recurrence of disease, alleviating symptoms, suppressing, diminishing or inhibiting any direct or indirect pathological consequences of the disease, ameliorating or palliating the disease state, and causing remission or improved prognosis. A “treatment” is intended to target the disease state and combat it, i.e., ameliorate or prevent the disease state. The particular treatment thus will depend on the disease state to be targeted and the current or future state of medicinal therapies and therapeutic approaches. A treatment may have associated toxicities.

As used herein, the term “truncated” may also be referred to herein as “shortened” or “without”.

As used herein, the term “variant” refers to a nucleotide sequence that, when compared to a reference sequence, contains at least one of a single nucleotide polymorphism, a single nucleotide variation, a conversion, an inversion, a duplication, a deletion, or a substitution. A “variant” includes amino acid sequences that derive from “variant” nucleotide sequences, as well as post-transcriptional and post-translational modifications thereto.

As considered herein, optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information website.

The nucleic acid and protein sequences of the present disclosure can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules provided in the disclosure. BLAST protein searches can be performed with the XBLAST program, score=50, word length=3 to obtain amino acid sequences homologous to the protein molecules of the disclosure. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

### Description of Aspects and Embodiments

In an aspect, a viral vector is provided comprising a therapeutic cargo portion, wherein the therapeutic cargo portion comprises a codon-optimized PAH sequence or variant thereof, a promoter, and an enhancer.

In an aspect, a viral vector is provided comprising a therapeutic cargo portion, wherein the therapeutic cargo portion comprises a codon-optimized PAH sequence or variant thereof and a promoter.

In an aspect, a viral vector is provided comprising a therapeutic cargo portion, wherein the therapeutic cargo portion comprises a codon-optimized PAH sequence or variant thereof and an enhancer.

In an aspect, a viral vector is provided comprising a therapeutic cargo portion, wherein the therapeutic cargo portion comprises a codon-optimized PAH sequence or variant thereof, a promoter, and a liver-specific enhancer.

In an aspect, a viral vector is provided comprising a therapeutic cargo portion, wherein the therapeutic cargo portion comprises a codon-optimized PAH sequence or variant thereof, a promoter, and a liver-specific enhancer, wherein the codon-optimized PAH sequence or variant thereof is operatively controlled by both the promoter and the liver-specific enhancer.

In an aspect, a viral vector is provided comprising a therapeutic cargo portion, wherein the therapeutic cargo portion comprises a codon-optimized PAH sequence or variant thereof and a promoter, wherein the codon-optimized PAH sequence or variant thereof is operatively controlled by the promoter.

In an aspect, a viral vector is provided comprising a therapeutic cargo portion, wherein the therapeutic cargo portion comprises a codon-optimized PAH sequence or variant thereof and an enhancer, wherein the codon-optimized PAH sequence or variant thereof is operatively controlled by the enhancer. In embodiments, the enhancer is a liver-specific enhancer.

In embodiments, any of the promoters described herein are at least one of a tissue-specific promoter, a constitutive promoter, and a synthetic promoter.

In embodiments, the tissue-specific promoter is a liver-specific promoter. In embodiments, the liver-specific promoter is a hAAT promoter. In embodiments, the hAAT promoter comprises a sequence having at least 75 percent, at least 80 percent, at least 85 percent, at least 90 percent, or at least 95 percent with SEQ ID NO: 4. For example, in embodiments, the hAAT promoter comprises a sequence that is 75 percent, 76 percent, 77 percent, 78 percent, 79 percent, 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent 97 percent, 98 percent, or 99 percent identical to SEQ ID NO: 4. In embodiments, the hAAT promoter comprises the sequence of SEQ ID NO: 4.

In embodiments, any of the liver-specific enhancers described herein are at least one of a naturally occurring enhancer and a synthetic enhancer.

In embodiments, the liver-specific enhancer is a prothrombin enhancer. In embodiments, the prothrombin enhancer comprises a sequence having at least 75 percent, at least 80 percent, at least 85 percent, at least 90 percent, or at least 95 percent identity with SEQ ID NO: 3. For example, in embodiments, the prothrombin enhancer comprises a sequence that is 75 percent, 76 percent, 77 percent, 78 percent, 79 percent, 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent, 97 percent, 98 percent, or 99 percent identical to SEQ ID NO: 3. In embodiments, the prothrombin enhancer comprises SEQ ID NO: 3.

In embodiments, the viral vector comprises an enhancer that is 5′ to a promoter. In embodiments, the viral vector comprises an enhancer that is 3′ to a promoter.

In embodiments, any of the codon-optimized PAH sequences or variants thereof are variants of a naturally occurring PAH sequence. In embodiments, any of the codon-optimized PAH sequences or variants thereof are variants of a synthetic PAH sequence.

In embodiments, the viral vector comprises a codon-optimized PAH sequence or variant thereof comprising a sequence having at least 75 percent, at least 80 percent, at least 85 percent, at least 90 percent, at least 95 percent sequence identity with SEQ ID NO: 70. For example, in embodiments, the codon-optimized PAH sequence is 75 percent, 76 percent, 77 percent, 78 percent, 79 percent, 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent, 97 percent, 98 percent, or 99 percent identical to SEQ ID NO: 70. In embodiments, the viral vector comprises a codon-optimized PAH sequence or variant thereof comprising the sequence of SEQ ID NO: 70. In embodiments, the codon-optimized PAH sequence or variant thereof comprises a sequence having 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%. 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% sequence identity with SEQ ID NO: 70.

In embodiments, any of the therapeutic cargo portions described herein further comprises an intron. In embodiments, the intron is derived from any plant or animal species. In embodiments, the intron is a beta globin intron. In embodiments, the beta globin intron is a human beta globin intron. In embodiments, the beta globin intron is a rabbit beta globin intron. In embodiments, the beta globin intron comprises a sequence having at least 75 percent, at least 80 percent, at least 85 percent, at least 90 percent, or at least 95 percent identity with SEQ ID NOS: 5 or 6. For example, in embodiments, the beta globin intron is 75 percent, 76 percent, 77 percent, 78 percent, 79 percent, 80 percent, 81 percent, 82 percent, 83 percent, 84 percent, 85 percent, 86 percent, 87 percent, 88 percent, 89 percent 90 percent, 91 percent, 92 percent, 93 percent, 94 percent, 95 percent, 96 percent 97 percent, 98 percent, or 99 percent identical to SEQ ID NOS: 5 or 6. In embodiments, the beta globin intron comprises the sequence of SEQ ID NOS: 5 or 6.

In embodiments, any of the therapeutic cargo portions described herein further comprise a site capable of being bound by a nuclear receptor. In embodiments, the nuclear receptor is expressed in the liver. In embodiments, the site is a hepatocyte nuclear factor binding site.

In embodiments, the hepatocyte nuclear factor binding site comprises a sequence having at least 75 percent, at least 80 percent, at least 85 percent, at least 90 percent, or at least 95 percent identity with SEQ ID NOS: 7, 8, 9, or 10. For example, in embodiments, the hepatocyte nuclear factor binding site is 75 percent, 76 percent, 77 percent, 78 percent, 79 percent, 80 percent, 81 percent, 82 percent, 83 percent, 84 percent, 85 percent 86 percent, 87 percent, 88 percent 89 percent, 90 percent, 91 percent, 92 percent, 93 percent, 94 percent, 95 percent, 96 percent, 97 percent, 98 percent, or 99 percent identical to SEQ ID NOS: 7, 8, 9, or 10. In embodiments, the hepatocyte nuclear factor binding site comprises the sequence of SEQ ID NOS: 7, 8, 9, or 10.

In embodiments, any of the hepatocyte nuclear factor binding sites described herein are disposed downstream of a prothrombin enhancer. In embodiments, any of the hepatocyte nuclear factor binding sites described herein are disposed upstream of a prothrombin enhancer. As used herein, downstream refers to a distance measured in contiguous nucleotide positions along the direction of transcription for the functional RNA. Upstream refers to a distance measured in contiguous positions opposite to the direction of transcription for the functional RNA.

In embodiments, any of the therapeutic cargo portions described herein further comprise at least one small RNA sequence that is capable of binding to at least one pre-determined PAH mRNA sequence.

In embodiments, any of the at least one small RNA described herein is a small nuclear RNA. In embodiments, the at least one small RNA is a small nucleolar RNA. In embodiments, the at least one small RNA, is a microRNA. In embodiments, the at least one small RNA is a small interfering RNA. In embodiments, the at least one small RNA is a short hairpin RNA.

In embodiments, the at least one small RNA sequence comprises a sequence having at least 75 percent, at least 80 percent, at least 85 percent, at least 90 percent, or at least 95 percent identity with SEQ ID NOS: 11 or 12. For example, in embodiments, the at least one small RNA sequence is 75 percent, 76 percent, 77 percent, 78 percent, 79 percent, 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent 97 percent, 98 percent, or 99 percent identical to SEQ ID NOS: 11 or 12. In embodiments, the at least one small RNA sequence comprises the sequence of SEQ ID NOS: 11 or 12.

In embodiments, any of the viral vectors described herein are at least one of a lentiviral vector and an AAV vector. In further embodiments, the following viral vectors can also be used in accordance with aspects of the present disclosure: Herpes simplex virus Type 1; Adenovirus, Moloney Murine Leukosis Virus; vectors based on oncoretroviruses including but not limited to HTLV-1 and HTLV-2; lentivirus vectors based on equine infectious anemia virus simian immunodeficiency virus, feline immunodeficiency virus, or Visna maedi lentivirus; measles virus vector; mumps virus vector; arbovirus vectors; equine infectious anemia virus vector; and vectors based on arenaviruses. In an aspect, gene delivery in accordance with the present disclosure may result in integration of a complementary gene copy at a location other than the gene encoding PAH, may result in creation of an extrachromosomal DNA or RNA element encoding PAH, may substitute for the natural PAH gene through homologous recombination, may utilize genome editing to insert a complementary gene sequence at or distant from the normal PAH gene or to exploit gene conversion to modify the sequence of chromosomal PAH genes. In another aspect, complementing DNA may be delivered in circular or linear forms through DNA transfection of liver, isolated hepatocytes or hepatocyte stem cells implanted into liver. In another aspect, complementing RNA may be delivered through transfection of liver, isolated hepatocytes or hepatocyte stem cells implanted into liver. In another aspect, isolated DNA or RNA may be delivered directly to accomplish gene conversion of the PAH gene, insert a complementing gene at a nearby or distant locus, or to modulate expression of negatively complementing chromosomal alleles of the PAH gene.

In an aspect, a viral vector is provided comprising a codon-optimized PAH sequence or variant thereof, wherein the codon-optimized sequence or variant thereof having at least 75 percent, at least 80 percent, at least 85 percent, at least 90 percent, or at least 95 percent sequence identity to SEQ ID NO: 71. For example, in embodiments, the codon-optimized PAH sequence or variant thereof is 75 percent, 76 percent, 77 percent, 78 percent, 79 percent, 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent 97 percent, 98 percent, or 99 percent identical to SEQ ID NO: 71. In embodiments, the codon-optimized sequence or variant thereof comprises the sequence of SEQ ID NO: 71. In embodiments, the codon-optimized PAH sequence or variant thereof comprises a sequence having 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%. 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% sequence identity with SEQ ID NO: 71.

In embodiments, the viral vector further comprises a therapeutic cargo portion that comprises the codon-optimized PAH sequence or variant thereof, a promoter, and a liver-specific enhancer, wherein the codon-optimized sequence or variant thereof is operatively controlled by both the promoter and the liver-specific enhancer.

In embodiments, the viral vector further comprises a therapeutic cargo portion that comprises the codon-optimized PAH sequence or variant thereof, a promoter, and an enhancer.

In embodiments, the promoter can be any promoter described herein. In embodiments, the enhancer can be any enhancer described herein.

In an aspect, a viral vector is provided comprising a codon-optimized PAH sequence or variant thereof, wherein the codon-optimized sequence or variant thereof having at least 75 percent, at least 80 percent, at least 85 percent, at least 90 percent, or at least 95 percent sequence identity to SEQ ID NO: 72. For example, in embodiments, the codon-optimized PAH sequence or variant thereof is 75 percent, 76 percent, 77 percent, 78 percent, 79 percent, 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent 97 percent, 98 percent, or 99 percent identical to SEQ ID NO: 72. In embodiments, the codon-optimized sequence or variant thereof comprises the sequence of SEQ ID NO: 72. In embodiments, the codon-optimized PAH sequence or variant thereof comprises a sequence having 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%. 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% sequence identity with SEQ ID NO: 72.

In embodiments, the viral vector further comprises a therapeutic cargo portion that comprises the codon-optimized PAH sequence or variant thereof, a promoter, and a liver-specific enhancer, wherein the codon-optimized sequence or variant thereof is operatively controlled by both the promoter and the liver-specific enhancer.

In embodiments, the viral vector further comprises a therapeutic cargo portion that comprises the codon-optimized PAH sequence or variant thereof, a promoter, and an enhancer.

In embodiments, the promoter can be any promoter described herein. In embodiments, the enhancer can be any enhancer described herein.

In an aspect, a viral vector is provided comprising a codon-optimized PAH sequence or variant thereof, wherein the codon-optimized PAH sequence or variant thereof having at least 75 percent, at least 80 percent, at least 85 percent, at least 90 percent, or at least 95 percent sequence identity to SEQ ID NO: 73. For example, in embodiments, the codon-optimized PAH sequence or variant thereof is 75 percent, 76 percent, 77 percent, 78 percent, 79 percent, 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent 97 percent, 98 percent, or 99 percent identical to SEQ ID NO: 73. In embodiments, the codon-optimized PAH sequence or variant thereof comprises the sequence of SEQ ID NO: 73. In embodiments, the codon-optimized PAH sequence or variant thereof comprises a sequence having 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%. 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% sequence identity with SEQ ID NO: 73.

In embodiments, the viral vector further comprises a therapeutic cargo portion that comprises the codon-optimized PAH sequence or variant thereof, a promoter, and a liver-specific enhancer, wherein the codon-optimized PAH sequence or variant thereof is operatively controlled by both the promoter and the liver-specific enhancer.

In embodiments, the viral vector further comprises a therapeutic cargo portion that comprises the codon-optimized PAH sequence or variant thereof, a promoter, and an enhancer.

In embodiments, the promoter can be any promoter described herein. In embodiments, the enhancer can be any enhancer described herein.

In an aspect, a viral vector is provided comprising a codon-optimized PAH sequence or variant thereof, wherein the codon-optimized PAH sequence or variant thereof having at least 75 percent, at least 80 percent, at least 85 percent, at least 90 percent, or at least 95 percent sequence identity to SEQ ID NO: 74. For example, in embodiments, the codon-optimized PAH sequence or variant thereof is 75 percent, 76 percent, 77 percent, 78 percent, 79 percent, 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent 97 percent, 98 percent, or 99 percent identical to SEQ ID NO: 74. In embodiments, the codon-optimized PAH sequence or variant thereof comprises the sequence of SEQ ID NO: 74. In embodiments, the codon-optimized PAH sequence or variant thereof comprises a sequence having 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%. 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% sequence identity with SEQ ID NO: 74.

In embodiments, the viral vector further comprises a therapeutic cargo portion that comprises the codon-optimized PAH sequence or variant thereof, a promoter, and a liver-specific enhancer, wherein the codon-optimized PAH sequence or variant thereof is operatively controlled by both the promoter and the liver-specific enhancer.

In embodiments, the viral vector further comprises a therapeutic cargo portion that comprises the codon-optimized PAH sequence or variant thereof, a promoter, and an enhancer.

In embodiments, the promoter can be any promoter described herein. In embodiments, the enhancer can be any enhancer described herein.

In an aspect, a viral vector is provided comprising a codon-optimized PAH sequence or variant thereof, wherein the codon-optimized PAH sequence or variant thereof having at least 75 percent, at least 80 percent, at least 85 percent, at least 90 percent, or at least 95 percent sequence identity to SEQ ID NO: 75. For example, in embodiments, the codon-optimized PAH sequence or variant thereof is 75 percent, 76 percent, 77 percent, 78 percent, 79 percent, 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent 97 percent, 98 percent, or 99 percent identical to SEQ ID NO: 75. In embodiments, the codon-optimized PAH sequence or variant thereof comprises the sequence of SEQ ID NO: 75. In embodiments, the codon-optimized PAH sequence or variant thereof comprises a sequence having 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%. 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% sequence identity with SEQ ID NO: 75.

In embodiments, the viral vector further comprises a therapeutic cargo portion that comprises a codon-optimized PAH sequence or variant thereof, a promoter, and a liver-specific enhancer, wherein the codon-optimized PAH sequence or variant thereof is operatively controlled by both the promoter and the liver-specific enhancer.

In embodiments, the viral vector further comprises a therapeutic cargo portion that comprises the codon-optimized PAH sequence or variant thereof, a promoter, and an enhancer.

In embodiments, the promoter can be any promoter described herein. In embodiments, the enhancer can be any enhancer described herein.

In an aspect, a viral vector is provided comprising a codon-optimized PAH sequence or variant thereof, wherein the codon-optimized PAH sequence or variant thereof having at least 75 percent, at least 80 percent, at least 85 percent, at least 90 percent, or at least 95 percent sequence identity to SEQ ID NO: 76. For example, in embodiments, the codon-optimized PAH sequence is 75 percent, 76 percent, 77 percent, 78 percent, 79 percent, 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent 97 percent, 98 percent, or 99 percent identical to SEQ ID NO: 76. In embodiments, the codon-optimized PAH sequence or variant thereof comprises the sequence of SEQ ID NO: 76. In embodiments, the codon-optimized PAH sequence or variant thereof comprises a sequence having 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%. 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% sequence identity with SEQ ID NO: 73.

In embodiments, the viral vector further comprises a therapeutic cargo portion that comprises a codon-optimized PAH sequence or variant thereof, a promoter, and a liver-specific enhancer, wherein the codon-optimized PAH sequence or variant thereof is operatively controlled by both the promoter and the liver-specific enhancer.

In embodiments, the viral vector further comprises a therapeutic cargo portion that comprises the codon-optimized PAH sequence or variant thereof, a promoter, and an enhancer.

In embodiments, the promoter can be any promoter described herein. In embodiments, the enhancer can be any enhancer described herein.

In an aspect, a viral vector is provided comprising a therapeutic cargo portion, wherein the therapeutic cargo portion comprises a codon-optimized PAH sequence or variant thereof comprising a sequence that shares greater than 90 percent sequence identity to SEQ ID NO: 70. For example, in embodiments, the codon-optimized PAH sequence or variant thereof is 91 percent, 92 percent, 93 percent, 94 percent, 95 percent, 96 percent, 97 percent, 98 percent, or 99 percent identical to SEQ ID NO: 70. In embodiments, the codon-optimized PAH sequence or variant thereof comprises SEQ ID NO: 70. In embodiments, the codon-optimized PAH sequence or variant thereof comprises a sequence having 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%. 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% sequence identity with SEQ ID NO: 70.

In an aspect, a viral vector is provided comprising a therapeutic cargo portion, wherein the therapeutic cargo portion comprises a codon-optimized PAH sequence or variant thereof comprising a sequence having at least 75 percent, at least 80 percent, at least 85 percent, at least 90 percent, or at least 95 sequence identity to SEQ ID NO 71. For example, in embodiments, the codon-optimized PAH sequence or variant thereof is 75 percent, 76 percent, 77 percent, 78 percent, 79 percent, 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent 97 percent, 98 percent, or 99 percent identical to SEQ ID NO: 71. In embodiments, the codon-optimized PAH sequence or variant thereof comprises SEQ ID NO: 71. In embodiments, the codon-optimized PAH sequence or variant thereof comprises a sequence having 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%. 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% sequence identity with SEQ ID NO: 71.

In an aspect, a viral vector is provided comprising a therapeutic cargo portion, wherein the therapeutic cargo portion comprises a codon-optimized PAH sequence or variant thereof comprising a sequence having at least 75 percent, at least 80 percent, at least 85 percent, at least 90 percent, or at least 95 sequence identity to SEQ ID NO: 72. For example, in embodiments, the codon-optimized PAH sequence or variant thereof is 75 percent, 76 percent, 77 percent, 78 percent, 79 percent, 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent 97 percent, 98 percent, or 99 percent identical to SEQ ID NO: 72. In embodiments the codon-optimized PAH sequence or variant thereof comprises SEQ ID NO: 72. In embodiments, the codon-optimized PAH sequence or variant thereof comprises a sequence having 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%. 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% sequence identity with SEQ ID NO: 72.

In an aspect, a viral vector is provided comprising a therapeutic cargo portion, wherein the therapeutic cargo portion comprises a codon-optimized PAH sequence or variant thereof comprising a sequence having at least 75 percent, at least 80 percent, at least 85 percent, at least 90 percent, or at least 95 percent sequence identity to SEQ ID NO: 73. For example, in embodiments, the codon-optimized PAH sequence or variant thereof is 75 percent, 76 percent, 77 percent, 78 percent, 79 percent, 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent 97 percent, 98 percent, or 99 percent identical to SEQ ID NO: 73. In embodiments, the codon-optimized PAH sequence or variant thereof comprises SEQ ID NO: 73. In embodiments, the codon-optimized PAH sequence or variant thereof comprises a sequence having 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%. 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% sequence identity with SEQ ID NO: 73.

In an aspect, a viral vector is provided comprising a therapeutic cargo portion, wherein the therapeutic cargo portion comprises a codon-optimized PAH sequence or variant thereof comprising a sequence having at least 75 percent, at least 80 percent, at least 85 percent, at least 90 percent, or at least 95 percent sequence identity to SEQ ID NO: 74. For example, in embodiments, the codon-optimized PAH sequence or variant thereof is 75 percent, 76 percent, 77 percent, 78 percent, 79 percent, 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent 97 percent, 98 percent, or 99 percent identical to SEQ ID NO: 74. In embodiments, the codon-optimized PAH sequence or variant thereof comprises SEQ ID NO: 74. In embodiments, the codon-optimized PAH sequence or variant thereof comprises a sequence having 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%. 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% sequence identity with SEQ ID NO: 74.

In an aspect, a viral vector is provided comprising a therapeutic cargo portion, wherein the therapeutic cargo portion comprises a codon-optimized PAH sequence or variant thereof comprising a sequence having at least 75 percent, at least 80 percent, at least 85 percent, at least 90 percent, or at least 95 percent sequence identity to SEQ ID NO: 75. For example, in embodiments, the codon-optimized PAH sequence or variant thereof is 75 percent, 76 percent, 77 percent, 78 percent, 79 percent, 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent 97 percent, 98 percent, or 99 percent identical to SEQ ID NO: 75. In embodiments, the codon-optimized PAH sequence or variant thereof comprises the sequence of SEQ ID NO: 75. In embodiments, the codon-optimized PAH sequence or variant thereof comprises a sequence having 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%. 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% sequence identity with SEQ ID NO: 75.

In an aspect, a viral vector is provided comprising a therapeutic cargo portion, wherein the therapeutic cargo portion comprises a codon-optimized PAH sequence or variant thereof comprising a sequence having at least 75 percent, at least 80 percent, at least 85 percent, at least 90 percent, or at least 95 percent sequence identity to SEQ ID NO: 76. For example, in embodiments, the codon-optimized PAH sequence or variant thereof is 75 percent, 76 percent, 77 percent, 78 percent, 79 percent, 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent 97 percent, 98 percent, or 99 percent identical to SEQ ID NO: 76. In embodiments, the codon-optimized PAH sequence or variant thereof comprises SEQ ID NO: 76. In embodiments, the codon-optimized PAH sequence or variant thereof comprises a sequence having 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%. 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% sequence identity with SEQ ID NO: 76.

In embodiments, the viral vector further comprises a therapeutic cargo portion that comprises the codon-optimized PAH sequence or variant thereof, and further comprises a promoter, and a liver-specific enhancer, wherein the codon-optimized PAH sequence or variant thereof is operatively controlled by both the promoter and the liver-specific enhancer.

In an aspect, a lentiviral particle produced by a packaging cell and capable of infecting a target cell is disclosed. In embodiments, the lentiviral particle comprises an envelope protein capable of infecting a target cell, and a viral vector as detailed herein.

In an aspect, a method of treating phenylketonuria (PKU) in a subject is disclosed. The method involves administering to the subject a therapeutically effective amount of a lentiviral particle as detailed herein.

In an aspect, use of a codon-optimized PAH sequence or variant thereof for treating PKU in a subject is provided. In another aspect, use of a codon-optimized PAH sequence or variant thereof to formulate a medicament for treating PKU in a subject is provided.

In an aspect, a codon-optimized PAH sequence or variant thereof for use in treating PKU in a subject is provided. In another aspect, a codon-optimized PAH sequence or variant thereof to formulate a medicament for use in treating PKU in a subject is provided.

In an aspect, a lentiviral vector is provided which enhances PAH sequence expression. In embodiments, at least one of a PAH sequence or PAH 3′UTR sequence is modified. In further embodiments, such modification alters the secondary structure of an mRNA transcript of the PAH sequence. In further embodiments, such modification comprises alteration of at least one of the mRNA PAH secondary structure sequence and the mRNA 3′ UTR secondary structure sequence. In further embodiments, such modification alters interactions of the coding region and 3′UTR region of PAH mRNA. In further embodiments, such modification inhibits the negative regulatory effects of PAH secondary structure on PAH protein production.

In embodiments, a modulated PAH sequence comprises any sequence in which the naturally occurring PAH sequence has been modified, including any addition, deletion, substitution, or modification of any one or more of its nucleotides, including any variants thereof. In embodiments, the modification comprises modulating one or more of the guanosine cytosine content of the naturally occurring sequence, one or more codons of the naturally occurring sequence, or one or more CpG sites of the naturally occurring sequence. In embodiments, the modification comprises a a codon-optimized PAH sequence. The PAH codon-optimized sequence may be any suitable PAH codon-optimized sequence, including those set forth and described herein. In embodiments, a vector that encodes a modified PAH sequence (including a codon-optimized sequence) results in higher PAH expression relative to a vector that encodes a PAH sequence that is not modified (e.g., that is not codon-optimized).

In embodiments, a modified PAH sequences comprises a sequence having at least 70%, 75%, 80%, at least 85%, at least 90%, or at least 95%, but less than 100%, sequence identity with any of SEQ ID NOs: 1, 70, 71 or 72. In embodiments the modified PAH comprises any of sequence of SEQ ID NOs: 70, 71 or 72.

In embodiments, a modulated PAH 3′UTR sequence comprises any sequence in which the naturally occurring PAH 3′ UTR sequence has been modified, including any addition, deletion, substitution, or modification of any one or more of its nucleotides, including any variants thereof. In embodiments, the modulated PAH 3′ UTR sequence comprises at least one of substitution or deletion of one or more of its nucleotides. In further embodiments all, or substantially all, of the 3′ UTR nucleotides are substituted or deleted.

In embodiments, the modified 3′UTR sequence comprises a 3′UTR sequence that is derived from a 3′UTR sequence of a different gene. In embodiments, the 3′UTR sequence of PAH is substituted with a 3′UTR sequence of a different gene. In embodiments, the 3′UTR sequence comprises albumin 3′UTR. In embodiments, the albumin 3′UTR comprises a sequence having at least 70%, 75%, 80%, at least 85%, at least 90%, or at least 95%, but less than 100%, sequence identity with SEQ ID NO: 86. In embodiments, the albumin 3′UTR comprises the sequence of SEQ ID NO: 86.

In embodiments, a lentiviral vector that encodes a PAH sequence that comprises a modified PAH 3′UTR sequence results in higher PAH expression than a lentiviral vector that encodes a PAH sequence in which the PAH 3′UTR is not disrupted.

In embodiments, a lentiviral vector that encodes a modified PAH 3′UTR and a modified PAH sequence (including a codon-optimized sequence) results in higher PAH expression relative to a vector that encodes any of PAH 3′UTR that is not modified and/or a PAH sequence that is not modified (e.g., that is not codon-optimized).

Phenylketonuria

PKU is believed to be caused by mutations of PAH and/or a defect in the synthesis or regeneration of PAH cofactors (i.e., BH₄). Notably, several PAH mutations have been shown to affect protein folding in the endoplasmic reticulum resulting in accelerated degradation and/or aggregation due to missense mutations (about 63%) and small deletions (about 13%) in protein structure that attenuates or largely abolishes enzyme catalytic activity. As there are numerous mutations that can affect the functionality of PAH, an effective therapeutic approach for treating PKU will need to address the aberrant PAH and a mode by which replacement PAH can be administered and/or generated.

In general, three major phenotypic groups are classified in PKU based on Phe levels measured at diagnosis, dietary tolerance to Phe and potential responsiveness to therapy. These groups include classical PKU (about Phe >1200 μM), atypical or mild PKU (Phe is about 600-1200 μM), and permanent mild hyperphenylalaninemia (HPA, Phe 120-600 μM).

Detection of PKU relies on universal newborn screening (NBS). A drop of blood collected from a heel stick is tested for phenylalanine levels in a screen that is mandatory in all 50 states of the USA and used routinely in most developed countries.

### Genetic Medicines

Genetic medicine includes reference to viral vectors that are used to deliver genetic constructs to host cells for the purposes of disease therapy or prevention.

Genetic constructs can include, but are not limited to, functional genes or portions of genes to correct or complement existing defects, DNA sequences encoding regulatory proteins, DNA sequences encoding regulatory RNA molecules including antisense, short hairpin RNA, short homology RNA, long non-coding RNA, small interfering RNA or others, and decoy sequences encoding either RNA or proteins designed to compete for critical cellular factors to alter a disease state. In embodiments, genetic medicine involves delivering these therapeutic genetic constructs to target cells to provide treatment or alleviation of a particular disease.

By delivering a functional PAH gene to the liver in vivo, PAH activity may be reconstituted leading to normal clearance of Phe in the blood therefore eliminating the need for dietary restrictions or frequent enzyme replacement therapies. The effect of this therapeutic approach may be improved by the targeting of a shRNA against endogenous PAN. In an aspect of the disclosure, a functional PAH gene or a variant thereof can also be delivered in utero if a fetus has been identified as being at risk to a PKU genotype. In embodiments, the functional PAH gene or a variant thereof is a codon-optimized PAH gene. In embodiments, the diagnostic step can be carried out to determine whether the fetus is at risk for a PKU phenotype. If the diagnostic step determines that the fetus is at risk for a PKU phenotype, then the fetus can be treated with the genetic medicines detailed herein. Treatment can occur in utero or in vitro.

### Lentiviral Vector System

A lentiviral virion (particle) in accordance with various aspects and embodiments herein is expressed by a vector system encoding the necessary viral proteins to produce a virion (viral particle). In various embodiments, one vector containing a nucleic acid sequence encoding the lentiviral Pol proteins is provided for reverse transcription and integration, operably linked to a promoter. In another embodiment, the Pol proteins are expressed by multiple vectors. In other embodiments, vectors containing a nucleic acid sequence encoding the lentiviral Gag proteins for forming a viral capsid, operably linked to a promoter, are provided. In embodiments, this gag nucleic acid sequence is on a separate vector than at least some of the pol nucleic acid sequence. In other embodiments, the gag nucleic acid sequence is on a separate vector from all the pol nucleic acid sequences that encode pol proteins.

Numerous modifications can be made to the vectors herein, which are used to create the particles to further minimize the chance of obtaining wild type revertants. These include, but are not limited to deletions of the U3 region of the LTR, tat deletions and matrix (MA) deletions. In embodiments, the gag, pol and env vector(s) do not contain nucleotides from the lentiviral genome that package lentiviral RNA, referred to as the lentiviral packaging sequence.

In embodiments, the vector(s) forming the particle do not contain a nucleic acid sequence from the lentiviral genome that expresses an envelope protein. In embodiments, a separate vector that contains a nucleic acid sequence encoding an envelope protein operably linked to a promoter is used. In embodiments, this separate vector encoding the envelop protein does not contain a lentiviral packaging sequence. In one embodiment the sequence encoding the envelope nucleic acid sequence encodes a lentiviral envelope protein.

In another embodiment the envelope protein is not from the lentivirus, but from a different virus. The resultant particle is referred to as a pseudotyped particle. By appropriate selection of envelopes one can “infect” virtually any cell. For example, one can use an env gene that encodes an envelope protein that targets an endocytic compartment. Examples of viruses from which such env genes and envelope proteins can derive include the influenza virus (e.g., the Influenza A virus, Influenza B virus, Influenza C virus, Influenza D virus, Isavirus, Quaranjavirus, and Thogotovirus), the Vesiculovirus (e.g., Indiana vesiculovirus), alpha viruses (e.g., the Semliki forest virus, Sindbis virus, Aura virus, Barmah Forest virus, Bebaru virus, Cabassou virus, Getah virus, Highlands J virus, Trocara virus, Una Virus, Ndumu virus, and Middleburg virus, among others), arenaviruses (e.g., the lymphocytic choriomeningitis virus, Machupo virus, Junin virus and Lassa Fever virus), flaviviruses (e.g., the tick-borne encephalitis virus, Dengue virus, hepatitis C virus, GB virus, Apoi virus, Bagaza virus, Edge Hill virus, Jugra virus, Kadam virus, Dakar bat virus, Modoc virus, Powassan virus, Usutu virus, and Sal Vieja virus, among others), rhabdoviruses (e.g., vesicular stomatitis virus, rabies virus), paramyxoviruses (e.g., mumps or measles) and orthomyxoviruses (e.g., influenza virus).

Other envelope proteins that can preferably be used include those derived from endogenous retroviruses (e.g., feline endogenous retroviruses and baboon endogenous retroviruses) and closely related gammaretroviruses (e.g., the Moloney Leukemia Virus, MLV-E, MLV-A, Gibbon Ape Leukemia Virus, GALV, Feline leukemia virus, Koala retrovirus, Trager duck spleen necrosis virus, Viper retrovirus, Chick syncytial virus, Gardner-Arnstein feline sarcoma virus, and Porcine type-C oncovirus, among others). These gammaretroviruses can be used as sources of env genes and envelope proteins for targeting primary cells. The gammaretroviruses are particularly preferred where the host cell is a primary cell.

Envelope proteins can be selected to target a specific desired host cell. For example, targeting specific receptors such as a dopamine receptor can be used for brain delivery. Another target can be vascular endothelium. These cells can be targeted using an envelope protein derived from any virus in the Filoviridae family (e.g., Cuevaviruses, Dianloviruses, Ebolaviruses, and Marburgviruses). Species of Ebolaviruses include Tai Forest ebolavirus, Zaire ebolavirus, Sudan ebolavirus, Bundibugyo ebolavirus, and Reston ebolavirus.

In addition, in embodiments, glycoproteins can undergo post-transcriptional modifications. For example, in an embodiment, the GP of Ebola, can be modified after translation to become the GP1 and GP2 glycoproteins. In another embodiment, one can use different lentiviral capsids with a pseudotyped envelope (e.g., FIV or SHIV [U.S. Pat. No. 5,654,195]). A SHIV pseudotyped vector can readily be used in animal models such as monkeys.

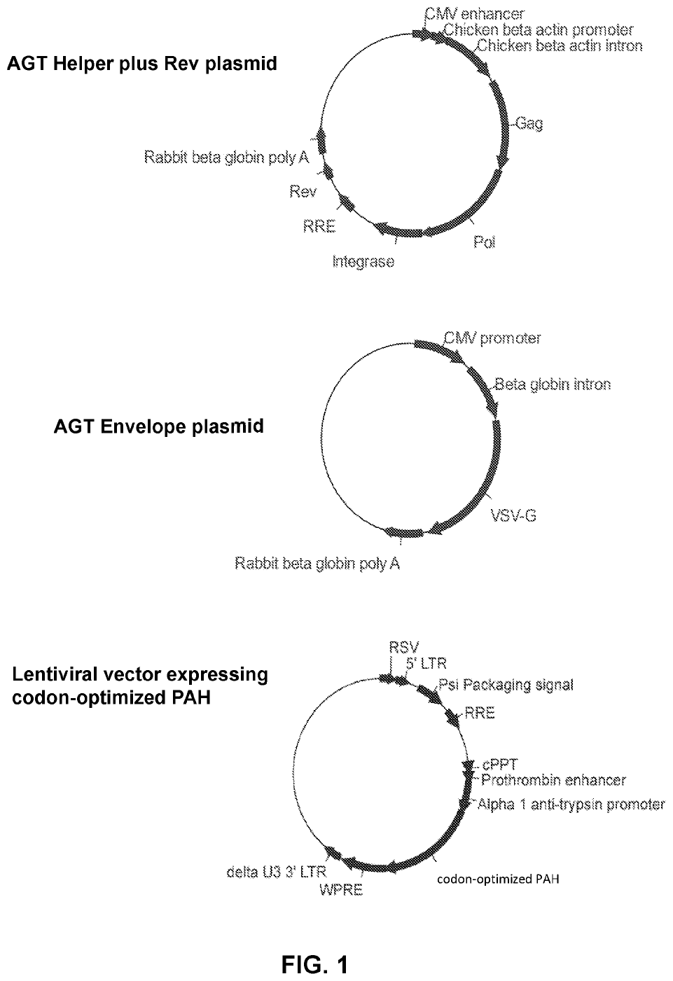

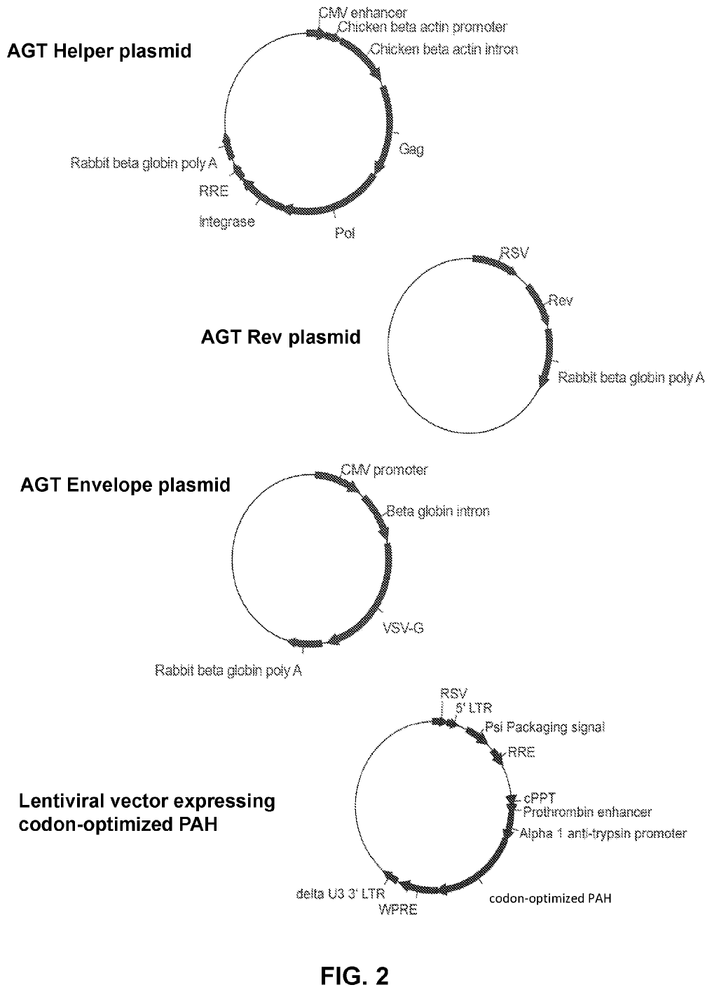

Lentiviral vector systems as provided herein typically include at least one helper plasmid comprising at least one of a gag, pol, or rev gene. Each of the gag, pol and rev genes may be provided on individual plasmids, or one or more genes may be provided together on the same plasmid. In one embodiment, the gag, pol, and rev genes are provided on the same plasmid (e.g., FIG. 1). In another embodiment, the gag and pol genes are provided on a first plasmid and the rev gene is provided on a second plasmid (e.g., FIG. 2). Accordingly, both 3-vector (e.g., FIG. 1) and 4-vector (e.g., FIG. 2) systems can be used to produce a lentivirus as described herein. In embodiments, the therapeutic vector, at least one envelope plasmid and at least one helper plasmid are transfected into a packaging cell, for example a packaging cell line. A non-limiting example of a packaging cell line is the 293T/17 HEK cell line. When the therapeutic vector, the envelope plasmid, and at least one helper plasmid are transfected into the packaging cell line, a lentiviral particle is ultimately produced. Lentiviral vector systems as provided herein typically include at least one helper plasmid comprising at least one of a gag, pol, or rev gene. Each of the gag, pol and rev genes may be provided on individual plasmids, or one or more genes may be provided together on the same plasmid. In one embodiment, the gag, pol, and rev genes are provided on the same plasmid (e.g., FIG. 1). In another embodiment, the gag and pol genes are provided on a first plasmid and the rev gene is provided on a second plasmid (e.g., FIG. 2). Accordingly, both 3-vector and 4-vector systems can be used to produce a lentivirus as described herein. In embodiments, the therapeutic vector, at least one envelope plasmid and at least one helper plasmid are transfected into a packaging cell, for example a packaging cell line. A non-limiting example of a packaging cell line is the 293T/17 HEK cell line. When the therapeutic vector, the envelope plasmid, and at least one helper plasmid are transfected into the packaging cell line, a lentiviral particle is ultimately produced.

In another aspect, a lentiviral vector system for expressing a lentiviral particle is disclosed. The system includes a lentiviral vector as described herein; an envelope plasmid for expressing an envelope protein optimized for infecting a cell; and at least one helper plasmid for expressing gag, pol, and rev genes, wherein when the lentiviral vector, the envelope plasmid, and the at least one helper plasmid are transfected into a packaging cell line, a lentiviral particle is produced by the packaging cell line, wherein the lentiviral particle is capable of inhibiting production of PAH.

In another aspect, the lentiviral vector, which is also referred to herein as a therapeutic vector, includes the following elements: hybrid 5′ long terminal repeat (Rous Sarcoma virus (RSV) promoter/5′ long terminal repeat (LTR)) (SEQ ID NOS: 13-14), Psi packaging signal (RNA packaging site) (SEQ ID NO: 15), Rev-response element (RRE) (SEQ ID NO: 16), central polypurine tract (cPPT) (polypurine tract) (SEQ ID NO: 17), human alpha-1 anti-trypsin promoter (hAAT) (SEQ ID NO: 4), Phenylalanine hydroxylase (PAH) (SEQ ID NOS: 1, 2, and 70-76), long Woodchuck Post-Transcriptional Regulatory Element (WPRE) sequence (SEQ ID NO: 18), and delta U3 3′ LTR (SEQ ID NO: 19). In embodiments, the lentiviral vector, which is also referred to herein as a therapeutic vector, includes the following elements: hybrid 5′ long terminal repeat (Rous Sarcoma virus (RSV) promoter/5′ long terminal repeat (LTR)) (SEQ ID NOS: 13-14), Psi packaging signal (RNA packaging site) (SEQ ID NO: 15), Rev-response element (RRE) (SEQ ID NO: 16), central polypurine tract (cPPT) (polypurine tract) (SEQ ID NO: 17), H1 promoter (SEQ ID NO: 20), PAH shRNA (SEQ ID NOS: 11 and 12), human alpha-1 anti-trypsin promoter (hAAT) (SEQ ID NO: 4), long Woodchuck Post-Transcriptional Regulatory Element (WPRE) sequence (SEQ ID NO: 18), and delta U3 3′ LTR (SEQ ID NO: 19). In embodiments, sequence variation, by way of substitution, deletion, addition, or mutation can be used to modify the sequences references herein.

In another aspect, a helper plasmid includes the following elements: CMV enhancer/chicken beta actin promoter (SEQ ID NO: 21); HIV component gag (SEQ ID NO: 22); HIV component pol (SEQ ID NO: 23); HIV Int (SEQ ID NO: 24); HIV RRE (SEQ ID NO: 25); and HIV Rev (SEQ ID NO: 26). In another aspect, the helper plasmid may be modified to include a first helper plasmid for expressing the gag gene (SEQ ID NO: 22) and pol gene (SEQ ID NO: 23), and a second and separate plasmid for expressing the rev gene (SEQ ID NO: 26). In embodiments, sequence variation, by way of substitution, deletion, addition, or mutation can be used to modify the sequences references herein.

In another aspect, an envelope plasmid includes the following elements: cytomegalovirus (CMV) promoter (SEQ ID NO: 27) and vesicular stomatitis virus G glycoprotein (VSV-G) (SEQ ID NO: 28). In embodiments, sequence variation, by way of substitution, deletion, addition, or mutation can be used to modify the sequences references herein.

In various aspects, the plasmids used for lentiviral packaging are modified by substitution, addition, subtraction or mutation of various elements without loss of vector function. For example, and without limitation, the following elements can replace similar elements in the plasmids that comprise the packaging system: Elongation Factor-1 alpha (EF-1 alpha) and ubiquitin C (UbC) promoters can replace the CMV or CAG promoter. SV40 poly A and bGH poly A can replace the rabbit beta globin poly A. In another aspect, the HIV sequences in the helper plasmid can be constructed from different HIV strains or clades. For example, the VSV-G glycoprotein can be substituted with membrane glycoproteins derived from gammaretroviruses (e.g., gibbon ape leukemia virus, GALV, murine leukemia virus 10A1, MLV, Koala retrovirus, Trager duck spleen necrosis virus, Viper retrovirus, Chick syncytial virus, Gardner-Arnstein feline sarcoma virus, and Porcine type-C oncovirus, among others), endogenous retroviruses (e.g., feline endogenous virus (RD114), human endogenous retrovirus such as HERV-W, and baboon endogenous retrovirus, BaEV, among others), Lyssavirus (e.g., Rabies virus, FUG), mammarenavirus (e.g., lymphocytic choriomeningitis virus, LCMV, Influenza viruses such as the Influenza A virus, Influenza A fowl plague virus, FPV, Influenza B virus, Influenza C virus, Influenza D virus, Isavirus, Quaranjavirus, and Thogotovirus), Alphavirus (e.g., Ross River alphavirus, RRV, or Ebola viruses, EboV, such as Sudan ebolavirus, Tai Forest ebolavirus, Zaire ebolavirus, Bundibugyo ebolavirus, and Reston ebolavirus).

Various lentiviral packaging systems can be acquired commercially (e.g., Lenti-vpak packaging kit from OriGene Technologies, Inc., Rockville, Md.), and can also be designed as described herein. Moreover, it is within the skill of a person ordinarily skilled in the relevant art to substitute or modify aspects of a lentiviral packaging system to improve any number of relevant factors, including the production efficiency of a lentiviral particle.

In another aspect, adeno-associated viral (AAV) vectors can also be used. In embodiments, the AAV vector is an AAV-DJ serotype. In embodiments, the AAV vector is any of serotypes 1-11. In embodiments, the AAV serotype is AAV-2. In embodiments, the AAV vector is a non-natural type engineered for optimal transduction of human hepatocytes.

AAV Vector Construction. In aspects of the disclosure, the PAH coding sequence (SEQ ID NOS: 1, 2, and 70-76) and the prothrombin enhancer (SEQ ID NO: 3) with hAAT promoter (SEQ ID NO: 4) are inserted into the pAAV plasmid (Cell Biolabs, San Diego, Calif.). The PAH coding sequence with flanking EcoRI and SalI restriction sites is synthesized by Eurofins Genomics (Louisville, Ky.). The pAAV plasmid and PAH sequence are digested with EcoRI and SalI enzyme and ligated together. Insertion of the PAH sequence is verified by sequencing. Next, the prothrombin enhancer and hAAT promoter are synthesized by Eurofins Genomics (Louisville, Ky.) with flanking MluI and EcoRI restriction sites. The pAAV plasmid containing the PAH coding sequence and the prothrombin enhancer/hAAT promoter sequence are digested with MluI and EcoRI enzymes and ligated together. Insertion of the prothrombin enhancer/hAAT promoter are verified by sequencing.

Further, a representative AAV plasmid system for expressing PAH may comprise an AAV Helper plasmid, an AAV plasmid, and an AAV Rev/Cap plasmid. The AAV Helper plasmid may contain a Left ITR (SEQ ID NO: 29), a Prothrombin enhancer (SEQ ID NO: 3), a human Anti alpha trypsin promoter (SEQ ID NO: 4), a PAH element (SEQ ID NOS: 1, 2 and 70-76), a PolyA element (SEQ ID NO: 30), and a Right ITR (SEQ ID NO: 31). The AAV plasmid may contain a suitable promoter element (SEQ ID NO: 21 or SEQ ID NO: 27), an E2A element (SEQ ID NO: 32), an E4 element (SEQ ID NO: 33), a viral associated (VA) RNA element (SEQ ID NO: 34), and a PolyA element (SEQ ID NO: 30). The AAV Rep/Cap plasmid may contain a suitable promoter element (SEQ ID NO: 21 or SEQ ID NO: 27), a Rep element (SEQ ID NO: 35; AAV2 Rep), a Cap element (SEQ ID NOS: 36 (AAV2 Cap), 37 (AAV8 Cap), or 38 (AAV DJ Cap)), and a PolyA element (SEQ ID NO: 30).

In embodiments, an AAV/DJ plasmid is provided comprising a prothrombin enhancer and a PAH sequence (AAV/DJ-Pro-PAH). In embodiments, the PAH sequence is any of the codon-optimized PAH sequences disclosed herein. In embodiments, an AAV/DJ plasmid is provided comprising a prothrombin enhancer, an intron, and a PAH sequence (AAV/DJ-Pro-Intron-PAH). In embodiments, the intron is a human beta globin intron. In embodiments, the intron is a rabbit beta globin intron. In embodiments, an AAV/DJ plasmid is provided comprising GFP (AAV/DJ-GFP).

In embodiments, an AAV2 plasmid is provided comprising a prothrombin enhancer and a PAH sequence (AAV2-Pro-PAH). In embodiments, the PAH sequence is any of the codon-optimized PAH sequences disclosed herein. In embodiments, an AAV2 plasmid is provided comprising a prothrombin enhancer, an intron, and a PAH sequence (AAV2-Pro-Intron-PAH). In embodiments, the intron is a human beta globin intron. In embodiments, the intron is a rabbit beta globin intron. In embodiments, an AAV2 is provided comprising GFP (AAV2-GFP).

In embodiments, any of the AAV vectors disclosed herein may contain a coding sequence that expresses a regulatory RNA. In embodiments, the regulatory RNA is a lncRNA. In embodiments, the regulatory RNA is a microRNA. In embodiments, the regulatory RNA is a piRNA. In embodiments, the regulatory RNA is a shRNA. In embodiments, the regulatory RNA is a small RNA sequence comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95% or more percent identity with SEQ ID NOS: 11 or 12.

Production of AAV particles. The AAV-PAH plasmid may be combined with the plasmids pAAV-RC2 (Cell Biolabs) and pHelper (Cell Biolabs). The pAAV-RC2 plasmid may contain the Rep and AAV-2 capsid genes and pHelper may contain the adenovirus E2A, E4, and VA genes. The AAV capsid may also comprise the AAV-8 (SEQ ID NO: 39) or AAV-DJ (SEQ ID NO: 40) sequences. To produce AAV particles, these plasmids may be transfected in the ratio 1:1:1 (pAAV-PAH: pAAV-RC2: pHelper) into 293T cells. For transfection of cells in 150 mm dishes (BD Falcon), 10 micrograms of each plasmid may be added together in 1 ml of DMEM. In another tube, 60 microliters of the transfection reagent PEI (1 microgram/ml) (Polysciences) may be added to 1 ml of DMEM. The two tubes may be mixed together and allowed to incubate for 15 minutes. Then the transfection mixture may be added to cells and the cells are collected after 3 days. The cells may be lysed by freeze/thaw lysis in dry ice/isopropanol. Benzonase nuclease (Sigma) may be added to the cell lysate for 30 minutes at 37 degrees Celsius. Cell debris may then be pelleted by centrifugation at 4 degrees Celsius for 15 minutes at 12,000 rpm. The supernatant may be collected and then added to target cells.

### Dosage and Dosage Forms

The disclosed compositions can be used for treating PKU patients during various stages of the disease. The disclosed vector compositions allow for short, medium, or long-term expression of genes or sequences of interest and episomal maintenance of the disclosed vectors. Accordingly, dosing regimens may vary based upon the condition being treated and the method of administration.

In embodiments, vector compositions may be administered to a subject in need in varying doses. Specifically, a subject may be administered about ≥10⁶ infectious doses (where 1 dose is needed on average to transduce 1 target cell). More specifically, a subject may be administered about ≥10⁷, about ≥10⁸, about ≥b 10⁹, about ≥10¹⁰, about ≥10¹¹, or about ≥10¹² infectious doses per kilogram of body weight, or any number of doses in-between these values. Upper limits of dosing will be determined for each disease indication, and will depend on toxicity/safety profiles for each individual product or product lot.

Additionally, vector compositions of the present disclosure may be administered periodically, such as once or twice a day, or any other suitable time period. For example, vector compositions may be administered to a subject in need once a week, once every other week, once every three weeks, once a month, every other month, every three months, every six months, every nine months, once a year, every eighteen months, every two years, every thirty months, or every three years.

In embodiments, the disclosed vector compositions are administered as a pharmaceutical composition. In embodiments, the pharmaceutical composition can be formulated in a wide variety of dosage forms, including but not limited to nasal, pulmonary, oral, topical, or parenteral dosage forms for clinical application. Each of the dosage forms can comprise various solubilizing agents, disintegrating agents, surfactants, fillers, thickeners, binders, diluents such as wetting agents or other pharmaceutically acceptable excipients. The pharmaceutical composition can also be formulated for injection, insufflation, infusion, or intradermal exposure. For instance, an injectable formulation may comprise the disclosed vectors in an aqueous or non-aqueous solution at a suitable pH and tonicity.

The disclosed vector compositions may be administered to a subject via direct injection into the liver with guided injection. In some embodiments, the vectors can be administered systemically via arterial or venous circulation. In some embodiments, the vector compositions can be administered via guided cannulation to tissues immediately surrounding liver including spleen or pancreas. In some embodiments, the vector compositions can be administered via guided cannulation or needle to kidney. In some embodiments, the vector compositions can be administered via guided cannulation or needle to specific regions of the brain including the substantia nigra. In some embodiments, the vector composition may be delivered by injection into the portal vein or portal sinus, and may be delivered by injection into the umbilical vein.

The disclosed vector compositions can be administered using any pharmaceutically acceptable method, such as intranasal, buccal, sublingual, oral, rectal, ocular, parenteral (intravenously, intradermally, intramuscularly, subcutaneously, intraperitoneally), pulmonary, intravaginal, locally administered, topically administered, topically administered after scarification, mucosally administered, via an aerosol, in semi-solid media such as agarose or gelatin, or via a buccal or nasal spray formulation.

Further, the disclosed vector compositions can be formulated into any pharmaceutically acceptable dosage form, such as a solid dosage form, tablet, pill, lozenge, capsule, liquid dispersion, gel, aerosol, pulmonary aerosol, nasal aerosol, ointment, cream, semi-solid dosage form, a solution, an emulsion, and a suspension. Further, the pharmaceutical composition may be a controlled release formulation, sustained release formulation, immediate release formulation, or any combination thereof. Further, the pharmaceutical composition may be a transdermal delivery system.

In embodiments, the pharmaceutical composition can be formulated in a solid dosage form for oral administration, and the solid dosage form can be powders, granules, capsules, tablets or pills. In embodiments, the solid dosage form can include one or more excipients such as calcium carbonate, starch, sucrose, lactose, microcrystalline cellulose or gelatin. In addition, the solid dosage form can include, in addition to the excipients, a lubricant such as talc or magnesium stearate. In some embodiments, the oral dosage form can be immediate release, or a modified release form. Modified release dosage forms include controlled or extended release, enteric release, and the like. The excipients used in the modified release dosage forms are commonly known to a person of ordinary skill in the art.

In embodiments, the pharmaceutical composition can be formulated as a sublingual or buccal dosage form. Such dosage forms comprise sublingual tablets or solution compositions that are administered under the tongue and buccal tablets that are placed between the cheek and gum.

In embodiments, the pharmaceutical composition can be formulated as a nasal dosage form. Such dosage forms of this disclosure comprise solution, suspension, and gel compositions for nasal delivery.

In embodiments, the pharmaceutical composition can be formulated in a liquid dosage form for oral administration, such as suspensions, emulsions or syrups. In embodiments, the liquid dosage form can include, in addition to commonly used simple diluents such as water and liquid paraffin, various excipients such as humectants, sweeteners, aromatics or preservatives. In embodiments, the composition can be formulated to be suitable for administration to a pediatric patient.

In embodiments, the pharmaceutical composition can be formulated in a dosage form for parenteral administration, such as sterile aqueous solutions, suspensions, emulsions, non-aqueous solutions or suppositories. In embodiments, the solutions or suspensions can include propylene glycol, polyethylene glycol, vegetable oils such as olive oil or injectable esters such as ethyl oleate.

The dosage of the pharmaceutical composition can vary depending on the patient's weight, age, gender, administration time and mode, excretion rate, and the severity of disease.

In embodiments, the treatment of PKU is accomplished by guided direct injection of the disclosed vector constructs into liver, using needle, or intravascular cannulation. In embodiments, the vectors compositions are administered into the cerebrospinal fluid, blood or lymphatic circulation by venous or arterial cannulation or injection, intradermal delivery, intramuscular delivery or injection into a draining organ near the liver.

The following examples are given to illustrate aspects of the present invention. It should be understood, however, that the inventions are not to be limited to the specific conditions or details described in these examples. All printed publications referenced herein are specifically incorporated by reference.

### EXAMPLES

### Example 1. Development of a Lentiviral Vector System

A lentiviral vector system was developed as summarized in FIG. 1 (circularized form).

Lentiviral particles were produced in 293T/17 HEK cells (purchased from American Type Culture Collection, Manassas, Va.) following transfection with the therapeutic vector, the envelope plasmid, and the helper plasmid. The transfection of 293T/17 HEK cells, which produced functional viral particles, employed the reagent Poly(ethylenimine) (PEI) to increase the efficiency of plasmid DNA uptake. The plasmids and DNA were initially added separately in culture medium without serum in a ratio of 3:1 (mass ratio of PEI to DNA). After 2-3 days, cell medium was collected and lentiviral particles were purified by high-speed centrifugation and/or filtration followed by anion-exchange chromatography. The concentration of lentiviral particles can be expressed in terms of transducing units/ml (TU/ml). The determination of TU was accomplished by measuring HIV p24 levels in culture fluids (p24 protein is incorporated into lentiviral particles), measuring the number of viral DNA copies per transduced cell by quantitative PCR, or by infecting cells and using light (if the vectors encode luciferase or fluorescent protein markers).

A 3-vector system (i.e., which includes a 2-vector lentiviral packaging system) was designed for the production of lentiviral particles. A schematic of the 3-vector system is shown in FIG. 1. Briefly, and with reference to FIG. 1, the top-most vector is a helper plasmid, which, in this case, includes Rev. The vector appearing in the middle of FIG. 1 is the envelope plasmid. The bottom-most vector is the therapeutic vector, as described herein.

Referring to FIG. 1, the Helper plus Rev plasmid includes a CMV enhancer/chicken beta actin promoter (SEQ ID NO: 21); a chicken beta actin intron (SEQ ID NO: 39); a HIV Gag (SEQ ID NO: 22); a HIV Pol (SEQ ID NO: 23); a HIV Integrase (SEQ ID NO: 24); a HIV RRE (SEQ ID NO: 25); a HIV Rev (SEQ ID NO: 26); and a rabbit beta globin poly A (SEQ ID NO: 40).

The envelope plasmid includes a CMV promoter (SEQ ID NO: 27); a beta globin intron (SEQ ID NO: 5 or 6); a VSV-G envelope glycoprotein (SEQ ID NO: 28); and a rabbit beta globin poly A (SEQ ID NO: 40).

Synthesis of a 3-vector system, which includes a 2-vector lentiviral packaging system containing the Helper (plus Rev) and Envelope plasmids, is disclosed.

Materials and Methods:

Construction of the helper plasmid: The helper plasmid was constructed by initial PCR amplification of a DNA fragment from the pNL4-3 HIV plasmid (NIH Aids Reagent Program) containing Gag, Pol, and Integrase genes. Primers were designed to amplify the fragment with EcoRI and NotI restriction sites which could be used to insert at the same sites in the pCDNA3 plasmid (Invitrogen). The forward primer was (5′-TAAGCAGAATTCATGAATTTGCCAGGAAGAT-3′) (SEQ ID NO: 41) and reverse primer was (5′-CCATACAATGAATGGACACTAGGCGGCCGCACGAAT-3′) (SEQ ID NO: 42).

The sequence for the Gag, Pol, Integrase fragment was as follows:

(SEQ ID NO: 43)
GAATTCATGAATTTGCCAGGAAGATGGAAACCAAA
AATGATAGGGGGAATTGGAGGTTTTATCAAAGTAA
GACAGTATGATCAGATACTCATAGAAATCTGCGGA
CATAAAGCTATAGGTACAGTATTAGTAGGACCTAC
ACCTGTCAACATAATTGGAAGAAATCTGTTGACTC
AGATTGGCTGCACTTTAAATTTTCCCATTAGTCCT
ATTGAGACTGTACCAGTAAAATTAAAGCCAGGAAT
GGATGGCCCAAAAGTTAAACAATGGCCATTGACAG
AAGAAAAAATAAAAGCATTAGTAGAAATTTGTACA
GAAATGGAAAAGGAAGGAAAAATTTCAAAAATTGG
GCCTGAAAATCCATACAATACTCCAGTATTTGCCA
TAAAGAAAAAAGACAGTACTAAATGGAGAAAATTA
GTAGATTTCAGAGAACTTAATAAGAGAACTCAAGA
TTTCTGGGAAGTTCAATTAGGAATACCACATCCTG
CAGGGTTAAAACAGAAAAAATCAGTAACAGTACTG
GATGTGGGCGATGCATATTTTTCAGTTCCCTTAGA
TAAAGACTTCAGGAAGTATACTGCATTTACCATAC
CTAGTATAAACAATGAGACACCAGGGATTAGATAT
CAGTACAATGTGCTTCCACAGGGATGGAAAGGATC
ACCAGCAATATTCCAGTGTAGCATGACAAAAATCT
TAGAGCCTTTTAGAAAACAAAATCCAGACATAGTC
ATCTATCAATACATGGATGATTTGTATGTAGGATC
TGACTTAGAAATAGGGCAGCATAGAACAAAAATAG
AGGAACTGAGACAACATCTGTTGAGGTGGGGATTT
ACCACACCAGACAAAAAACATCAGAAAGAACCTCC
ATTCCTTTGGATGGGTTATGAACTCCATCCTGATA
AATGGACAGTACAGCCTATAGTGCTGCCAGAAAAG
GACAGCTGGACTGTCAATGACATACAGAAATTAGT
GGGAAAATTGAATTGGGCAAGTCAGATTTATGCAG
GGATTAAAGTAAGGCAATTATGTAAACTTCTTAGG
GGAACCAAAGCACTAACAGAAGTAGTACCACTAAC
AGAAGAAGCAGAGCTAGAACTGGCAGAAAACAGGG
AGATTCTAAAAGAACCGGTACATGGAGTGTATTAT
GACCCATCAAAAGACTTAATAGCAGAAATACAGAA
GCAGGGGCAAGGCCAATGGACATATCAAATTTATC
AAGAGCCATTTAAAAATCTGAAAACAGGAAAGTAT
GCAAGAATGAAGGGTGCCCACACTAATGATGTGAA
ACAATTAACAGAGGCAGTACAAAAAATAGCCACAG
AAAGCATAGTAATATGGGGAAAGACTCCTAAATTT
AAATTACCCATACAAAAGGAAACATGGGAAGCATG
GTGGACAGAGTATTGGCAAGCCACCTGGATTCCTG
AGTGGGAGTTTGTCAATACCCCTCCCTTAGTGAAG
TTATGGTACCAGTTAGAGAAAGAACCCATAATAGG
AGCAGAAACTTTCTATGTAGATGGGGCAGCCAATA
GGGAAACTAAATTAGGAAAAGCAGGATATGTAACT
GACAGAGGAAGACAAAAAGTTGTCCCCCTAACGGA
CACAACAAATCAGAAGACTGAGTTACAAGCAATTC
ATCTAGCTTTGCAGGATTCGGGATTAGAAGTAAAC
ATAGTGACAGACTCACAATATGCATTGGGAATCAT
TCAAGCACAACCAGATAAGAGTGAATCAGAGTTAG
TCAGTCAAATAATAGAGCAGTTAATAAAAAAGGAA
AAAGTCTACCTGGCATGGGTACCAGCACACAAAGG
AATTGGAGGAAATGAACAAGTAGATAAATTGGTCA
GTGCTGGAATCAGGAAAGTACTATTTTTAGATGGA
ATAGATAAGGCCCAAGAAGAACATGAGAAATATCA
CAGTAATTGGAGAGCAATGGCTAGTGATTTTAACC
TACCACCTGTAGTAGCAAAAGAAATAGTAGCCAGC
TGTGATAAATGTCAGCTAAAAGGGGAAGCCATGCA
TGGACAAGTAGACTGTAGCCCAGGAATATGGCAGC
TAGATTGTACACATTTAGAAGGAAAAGTTATCTTG
GTAGCAGTTCATGTAGCCAGTGGATATATAGAAGC
AGAAGTAATTCCAGCAGAGACAGGGCAAGAAACAG
CATACTTCCTCTTAAAATTAGCAGGAAGATGGCCA
GTAAAAACAGTACATACAGACAATGGCAGCAATTT
CACCAGTACTACAGTTAAGGCCGCCTGTTGGTGGG
CGGGGATCAAGCAGGAATTTGGCATTCCCTACAAT
CCCCAAAGTCAAGGAGTAATAGAATCTATGAATAA
AGAATTAAAGAAAATTATAGGACAGGTAAGAGATC
AGGCTGAACATCTTAAGACAGCAGTACAAATGGCA
GTATTCATCCACAATTTTAAAAGAAAAGGGGGGAT
TGGGGGGTACAGTGCAGGGGAAAGAATAGTAGACA
TAATAGCAACAGACATACAAACTAAAGAATTACAA
AAACAAATTACAAAAATTCAAAATTTTCGGGTTTA
TTACAGGGACAGCAGAGATCCAGTTTGGAAAGGAC
CAGCAAAGCTCCTCTGGAAAGGTGAAGGGGCAGTA
GTAATACAAGATAATAGTGACATAAAAGTAGTGCC
AAGAAGAAAAGCAAAGATCATCAGGGATTATGGAA
AACAGATGGCAGGTGATGATTGTGTGGCAAGTAGA
CAGGATGAGGATTAA.

Next, a DNA fragment containing the RRE, Rev, and rabbit beta globin poly A sequence with XbaI and XmaI flanking restriction sites was synthesized by Eurofins Genomics. The DNA fragment was then inserted into the plasmid at the XbaI and XmaI restriction sites The DNA sequence was as follows:

(SEQ ID NO: 44)
TCTAGAATGGCAGGAAGAAGCGGAGACAGCGACGA
AGAGCTCATCAGAACAGTCAGACTCATCAAGCTTC
TCTATCAAAGCAACCCACCTCCCAATCCCGAGGGG
ACCCGACAGGCCCGAAGGAATAGAAGAAGAAGGTG
GAGAGAGAGACAGAGACAGATCCATTCGATTAGTG
AACGGATCCTTGGCACTTATCTGGGACGATCTGCG
GAGCCTGTGCCTCTTCAGCTACCACCGCTTGAGAG
ACTTACTCTTGATTGTAACGAGGATTGTGGAACTT
CTGGGACGCAGGGGGTGGGAAGCCCTCAAATATTG
GTGGAATCTCCTACAATATTGGAGTCAGGAGCTAA
AGAATAGAGGAGCTTTGTTCCTTGGGTTCTTGGGA
GCAGCAGGAAGCACTATGGGCGCAGCGTCAATGAC
GCTGACGGTACAGGCCAGACAATTATTGTCTGGTA
TAGTGCAGCAGCAGAACAATTTGCTGAGGGCTATT
GAGGCGCAACAGCATCTGTTGCAACTCACAGTCTG
GGGCATCAAGCAGCTCCAGGCAAGAATCCTGGCTG
TGGAAAGATACCTAAAGGATCAACAGCTCCTAGAT
CTTTTTCCCTCTGCCAAAAATTATGGGGACATCAT
GAAGCCCCTTGAGCATCTGACTTCTGGCTAATAAA
GGAAATTTATTTTCATTGCAATAGTGTGTTGGAAT
TTTTTGTGTCTCTCACTCGGAAGGACATATGGGAG
GGCAAATCATTTAAAACATCAGAATGAGTATTTGG
TTTAGAGTTTGGCAACATATGCCATATGCTGGCTG
CCATGAACAAAGGTGGCTATAAAGAGGTCATCAGT
ATATGAAACAGCCCCCTGCTGTCCATTCCTTATTC
CATAGAAAAGCCTTGACTTGAGGTTAGATTTTTTT
TATATTTTGTTTTGTGTTATTTTTTTCTTTAACAT
CCCTAAAATTTTCCTTACATGTTTTACTAGCCAGA
TTTTTCCTCCTCTCCTGACTACTCCCAGTCATAGC
TGTCCCTCTTCTCTTATGAAGATCCCTCGACCTGC
AGCCCAAGCTTGGCGTAATCATGGTCATAGCTGTT
TCCTGTGTGAAATTGTTATCCGCTCACAATTCCAC
ACAACATACGAGCCGGAAGCATAAAGTGTAAAGCC
TGGGGTGCCTAATGAGTGAGCTAACTCACATTAAT
TGCGTTGCGCTCACTGCCCGCTTTCCAGTCGGGAA
ACCTGTCGTGCCAGCGGATCCGCATCTCAATTAGT
CAGCAACCATAGTCCCGCCCCTAACTCCGCCCATC
CCGCCCCTAACTCCGCCCAGTTCCGCCCATTCTCC
GCCCCATGGCTGACTAATTTTTTTTATTTATGCAG
AGGCCGAGGCCGCCTCGGCCTCTGAGCTATTCCAG
AAGTAGTGAGGAGGCTTTTTTGGAGGCCTAGGCTT
TTGCAAAAAGCTAACTTGTTTATTGCAGCTTATAA
TGGTTACAAATAAAGCAATAGCATCACAAATTTCA
CAAATAAAGCATTTTTTTCACTGCATTCTAGTTGT
GGTTTGTCCAAACTCATCAATGTATCTTATCAGCG
GCCGCCCCGGG

Finally, the CMV promoter of pCDNA3.1 was replaced with the CAG promoter (CMV enhancer, chicken beta actin promoter plus a chicken beta actin intron sequence). A DNA fragment containing the CAG enhancer/promoter/intron sequence with MluI and EcoRI flanking restriction sites was synthesized by Eurofins Genomics. The DNA fragment was then inserted into the plasmid at the MluI and EcoRI restriction sites. The DNA sequence was as follows:

(SEQ ID NO: 45)
ACGCGTTAGTTATTAATAGTAATCAATTACGGGGT
CATTAGTTCATAGCCCATATATGGAGTTCCGCGTT
ACATAACTTACGGTAAATGGCCCGCCTGGCTGACC
GCCCAACGACCCCCGCCCATTGACGTCAATAATGA
CGTATGTTCCCATAGTAACGCCAATAGGGACTTTC
CATTGACGTCAATGGGTGGACTATTTACGGTAAAC
TGCCCACTTGGCAGTACATCAAGTGTATCATATGC
CAAGTACGCCCCCTATTGACGTCAATGACGGTAAA
TGGCCCGCCTGGCATTATGCCCAGTACATGACCTT
ATGGGACTTTCCTACTTGGCAGTACATCTACGTAT
TAGTCATCGCTATTACCATGGGTCGAGGTGAGCCC
CACGTTCTGCTTCACTCTCCCCATCTCCCCCCCCT
CCCCACCCCCAATTTTGTATTTATTTATTTTTTAA
TTATTTTGTGCAGCGATGGGGGCGGGGGGGGGGGG
GGCGCGCGCCAGGCGGGGCGGGGCGGGGCGAGGGG
CGGGGCGGGGCGAGGCGGAGAGGTGCGGCGGCAGC
CAATCAGAGCGGCGCGCTCCGAAAGTTTCCTTTTA
TGGCGAGGCGGCGGCGGCGGCGGCCCTATAAAAAG
CGAAGCGCGCGGCGGGCGGGAGTCGCTGCGTTGCC
TTCGCCCCGTGCCCCGCTCCGCGCCGCCTCGCGCC
GCCCGCCCCGGCTCTGACTGACCGCGTTACTCCCA
CAGGTGAGCGGGCGGGACGGCCCTTCTCCTCCGGG
CTGTAATTAGCGCTTGGTTTAATGACGGCTCGTTT
CTTTTCTGTGGCTGCGTGAAAGCCTTAAAGGGCTC
CGGGAGGGCCCTTTGTGCGGGGGGGAGCGGCTCGG
GGGGTGCGTGCGTGTGTGTGTGCGTGGGGAGCGCC
GCGTGCGGCCCGCGCTGCCCGGCGGCTGTGAGCGC
TGCGGGCGCGGCGCGGGGCTTTGTGCGCTCCGCGT
GTGCGCGAGGGGAGCGCGGCCGGGGGCGGTGCCCC
GCGGTGCGGGGGGGCTGCGAGGGGAACAAAGGCTG
CGTGCGGGGTGTGTGCGTGGGGGGGTGAGCAGGGG
GTGTGGGCGCGGCGGTCGGGCTGTAACCCCCCCCT
GCACCCCCCTCCCCGAGTTGCTGAGCACGGCCCGG
CTTCGGGTGCGGGGCTCCGTGCGGGGCGTGGCGCG
GGGCTCGCCGTGCCGGGCGGGGGGTGGCGGCAGGT
GGGGGTGCCGGGCGGGGCGGGGCCGCCTCGGGCCG
GGGAGGGCTCGGGGGAGGGGCGCGGCGGCCCCGGA
GCGCCGGCGGCTGTCGAGGCGCGGCGAGCCGCAGC
CATTGCCTTTTATGGTAATCGTGCGAGAGGGCGCA
GGGACTTCCTTTGTCCCAAATCTGGCGGAGCCGAA
ATCTGGGAGGCGCCGCCGCACCCCCTCTAGCGGGC
GCGGGCGAAGCGGTGCGGCGCCGGCAGGAAGGAAA
TGGGCGGGGAGGGCCTTCGTGCGTCGCCGCGCCGC
CGTCCCCTTCTCCATCTCCAGCCTCGGGGCTGCCG
CAGGGGGACGGCTGCCTTCGGGGGGGACGGGGCAG
GGCGGGGTTCGGCTTCTGGCGTGTGACCGGCGGGA
ATTC

Construction of the VSV-Envelope Plasmid:

The vesicular stomatitis Indiana virus glycoprotein (VSV-G) sequence was synthesized by Eurofins Genomics with flanking EcoRI restriction sites. The DNA fragment was then inserted into the pCDNA3.1 plasmid (Invitrogen) at the EcoRI restriction site and the correct orientation was determined by sequencing using a CMV specific primer.

The DNA sequence was as follows:

(SEQ ID NO: 28)
ATGAAGTGCCTTTTGTACTTAGCCTTTTTATTCAT
TGGGGTGAATTGCAAGTTCACCATAGTTTTTCCAC
ACAACCAAAAAGGAAACTGGAAAAATGTTCCTTCT
AATTACCATTATTGCCCGTCAAGCTCAGATTTAAA
TTGGCATAATGACTTAATAGGCACAGCCTTACAAG
TCAAAATGCCCAAGAGTCACAAGGCTATTCAAGCA
GACGGTTGGATGTGTCATGCTTCCAAATGGGTCAC
TACTTGTGATTTCCGCTGGTATGGACCGAAGTATA
TAACACATTCCATCCGATCCTTCACTCCATCTGTA
GAACAATGCAAGGAAAGCATTGAACAAACGAAACA
AGGAACTTGGCTGAATCCAGGCTTCCCTCCTCAAA
GTTGTGGATATGCAACTGTGACGGATGCCGAAGCA
GTGATTGTCCAGGTGACTCCTCACCATGTGCTGGT
TGATGAATACACAGGAGAATGGGTTGATTCACAGT
TCATCAACGGAAAATGCAGCAATTACATATGCCCC
ACTGTCCATAACTCTACAACCTGGCATTCTGACTA
TAAGGTCAAAGGGCTATGTGATTCTAACCTCATTT
CCATGGACATCACCTTCTTCTCAGAGGACGGAGAG
CTATCATCCCTGGGAAAGGAGGGCACAGGGTTCAG
AAGTAACTACTTTGCTTATGAAACTGGAGGCAAGG
CCTGCAAAATGCAATACTGCAAGCATTGGGGAGTC
AGACTCCCATCAGGTGTCTGGTTCGAGATGGCTGA
TAAGGATCTCTTTGCTGCAGCCAGATTCCCTGAAT
GCCCAGAAGGGTCAAGTATCTCTGCTCCATCTCAG
ACCTCAGTGGATGTAAGTCTAATTCAGGACGTTGA
GAGGATCTTGGATTATTCCCTCTGCCAAGAAACCT
GGAGCAAAATCAGAGCGGGTCTTCCAATCTCTCCA
GTGGATCTCAGCTATCTTGCTCCTAAAAACCCAGG
AACCGGTCCTGCTTTCACCATAATCAATGGTACCC
TAAAATACTTTGAGACCAGATACATCAGAGTCGAT
ATTGCTGCTCCAATCCTCTCAAGAATGGTCGGAAT
GATCAGTGGAACTACCACAGAAAGGGAACTGTGGG
ATGACTGGGCACCATATGAAGACGTGGAAATTGGA
CCCAATGGAGTTCTGAGGACCAGTTCAGGATATAA
GTTTCCTTTATACATGATTGGACATGGTATGTTGG
ACTCCGATCTTCATCTTAGCTCAAAGGCTCAGGTG
TTCGAACATCCTCACATTCAAGACGCTGCTTCGCA
ACTTCCTGATGATGAGAGTTTATTTTTTGGTGATA
CTGGGCTATCCAAAAATCCAATCGAGCTTGTAGAA
GGTTGGTTCAGTAGTTGGAAAAGCTCTATTGCCTC
TTTTTTCTTTATCATAGGGTTAATCATTGGACTAT
TCTTGGTTCTCCGAGTTGGTATCCATCTTTGCATT
AAATTAAAGCACACCAAGAAAAGACAGATTTATAC
AGACATAGAGATGAACCGACTTGGAAAGTGA

A 4-vector system, which includes a 3-vector lentiviral packaging system, has also been designed and produced using the methods and materials described herein. A schematic of the 4-vector system is shown in FIG. 2. Briefly, and with reference to FIG. 2, the top-most vector is a helper plasmid, which, in this case, does not include Rev. The second vector is a separate Rev plasmid. The third vector is the envelope plasmid. The bottom-most vector is the therapeutic vector as described herein.

Referring to FIG. 2, the Helper plasmid includes a CMV enhancer/chicken beta actin promoter (SEQ ID NO: 21); a chicken beta actin intron (SEQ ID NO: 39); a HIV Gag (SEQ ID NO: 22); a HIV Pol (SEQ ID NO: 23); a HIV Integrase (SEQ ID NO: 24); a HIV RRE (SEQ ID NO: 25); and a rabbit beta globin poly A (SEQ ID NO: 40).

The Rev plasmid includes a RSV promoter and HIV Rev (SEQ ID NO: 46); and a rabbit beta globin poly A (SEQ ID NO: 40).

The Envelope plasmid includes a CMV promoter (SEQ ID NO: 27); a beta globin intron (SEQ ID NO: 5 or 6); a VSV-G envelope glycoprotein (SEQ ID NO: 28); and a rabbit beta globin poly A (SEQ ID NO: 40).

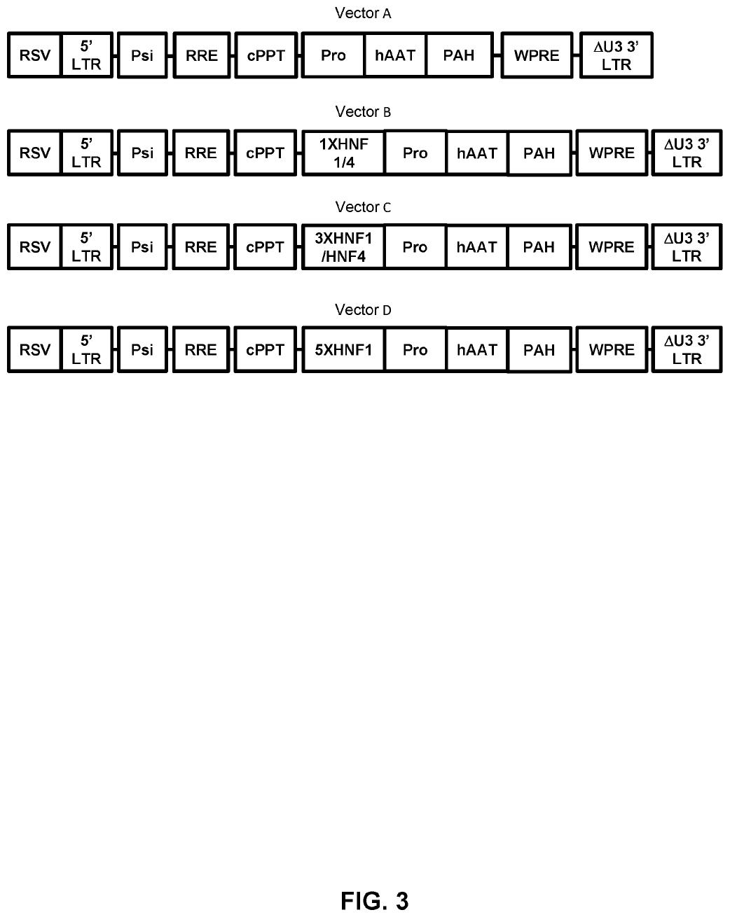

In one aspect, the therapeutic lentiviral vector expressing PAH includes all of the elements shown in Vector A of FIG. 3. In another aspect, the therapeutic lentiviral vector expressing PAH includes all of the elements shown in Vector B of FIG. 3. In another aspect, the therapeutic lentiviral vector expressing PAH includes all of the elements shown in Vector C of FIG. 3. In another aspect, the therapeutic lentiviral vector expressing PAH includes all of the elements shown in Vector D of FIG. 3.

Synthesis of a 4-vector system, which includes a 3-vector lentiviral packaging system containing the Helper, Rev, and Envelope plasmids, is disclosed.

Materials and Methods:

Construction of the Helper Plasmid without Rev:

The Helper plasmid without Rev was constructed by inserting a DNA fragment containing the RRE and rabbit beta globin poly A sequence. This sequence was synthesized by Eurofins Genomics with flanking XbaI and XmaI restriction sites. The RRE/rabbit poly A beta globin sequence was then inserted into the Helper plasmid at the XbaI and XmaI restriction sites.

The DNA sequence is as follows:

(SEQ ID NO: 44)
TCTAGAATGGCAGGAAGAAGCGGAGACAGCGACGA
AGAGCTCATCAGAACAGTCAGACTCATCAAGCTTC
TCTATCAAAGCAACCCACCTCCCAATCCCGAGGGG
ACCCGACAGGCCCGAAGGAATAGAAGAAGAAGGTG
GAGAGAGAGACAGAGACAGATCCATTCGATTAGTG
AACGGATCCTTGGCACTTATCTGGGACGATCTGCG
GAGCCTGTGCCTCTTCAGCTACCACCGCTTGAGAG
ACTTACTCTTGATTGTAACGAGGATTGTGGAACTT
CTGGGACGCAGGGGGTGGGAAGCCCTCAAATATTG
GTGGAATCTCCTACAATATTGGAGTCAGGAGCTAA
AGAATAGAGGAGCTTTGTTCCTTGGGTTCTTGGGA
GCAGCAGGAAGCACTATGGGCGCAGCGTCAATGAC
GCTGACGGTACAGGCCAGACAATTATTGTCTGGTA
TAGTGCAGCAGCAGAACAATTTGCTGAGGGCTATT
GAGGCGCAACAGCATCTGTTGCAACTCACAGTCTG
GGGCATCAAGCAGCTCCAGGCAAGAATCCTGGCTG
TGGAAAGATACCTAAAGGATCAACAGCTCCTAGAT
CTTTTTCCCTCTGCCAAAAATTATGGGGACATCAT
GAAGCCCCTTGAGCATCTGACTTCTGGCTAATAAA
GGAAATTTATTTTCATTGCAATAGTGTGTTGGAAT
TTTTTGTGTCTCTCACTCGGAAGGACATATGGGAG
GGCAAATCATTTAAAACATCAGAATGAGTATTTGG
TTTAGAGTTTGGCAACATATGCCATATGCTGGCTG
CCATGAACAAAGGTGGCTATAAAGAGGTCATCAGT
ATATGAAACAGCCCCCTGCTGTCCATTCCTTATTC
CATAGAAAAGCCTTGACTTGAGGTTAGATTTTTTT
TATATTTTGTTTTGTGTTATTTTTTTCTTTAACAT
CCCTAAAATTTTCCTTACATGTTTTACTAGCCAGA
TTTTTCCTCCTCTCCTGACTACTCCCAGTCATAGC
TGTCCCTCTTCTCTTATGAAGATCCCTCGACCTGC
AGCCCAAGCTTGGCGTAATCATGGTCATAGCTGTT
TCCTGTGTGAAATTGTTATCCGCTCACAATTCCAC
ACAACATACGAGCCGGAAGCATAAAGTGTAAAGCC
TGGGGTGCCTAATGAGTGAGCTAACTCACATTAAT
TGCGTTGCGCTCACTGCCCGCTTTCCAGTCGGGAA
ACCTGTCGTGCCAGCGGATCCGCATCTCAATTAGT
CAGCAACCATAGTCCCGCCCCTAACTCCGCCCATC
CCGCCCCTAACTCCGCCCAGTTCCGCCCATTCTCC
GCCCCATGGCTGACTAATTTTTTTTATTTATGCAG
AGGCCGAGGCCGCCTCGGCCTCTGAGCTATTCCAG
AAGTAGTGAGGAGGCTTTTTTGGAGGCCTAGGCTT
TTGCAAAAAGCTAACTTGTTTATTGCAGCTTATAA
TGGTTACAAATAAAGCAATAGCATCACAAATTTCA
CAAATAAAGCATTTTTTTCACTGCATTCTAGTTGT
GGTTTGTCCAAACTCATCAATGTATCTTATCAGCG
GCCGCCCCGGG

Construction of the Rev Plasmid:

The RSV promoter and HIV Rev sequences were synthesized as a single DNA fragment by Eurofins Genomics with flanking MfeI and XbaI restriction sites. The DNA fragment was then inserted into the pCDNA3.1 plasmid (Invitrogen) at the MfeI and XbaI restriction sites in which the CMV promoter is replaced with the RSV promoter. The DNA sequence was as follows:

(SEQ ID NO: 46)
CAATTGCGATGTACGGGCCAGATATACGCGTATCT
GAGGGGACTAGGGTGTGTTTAGGCGAAAAGCGGGG
CTTCGGTTGTACGCGGTTAGGAGTCCCCTCAGGAT
ATAGTAGTTTCGCTTTTGCATAGGGAGGGGGAAAT
GTAGTCTTATGCAATACACTTGTAGTCTTGCAACA
TGGTAACGATGAGTTAGCAACATGCCTTACAAGGA
GAGAAAAAGCACCGTGCATGCCGATTGGTGGAAGT
AAGGTGGTACGATCGTGCCTTATTAGGAAGGCAAC
AGACAGGTCTGACATGGATTGGACGAACCACTGAA
TTCCGCATTGCAGAGATAATTGTATTTAAGTGCCT
AGCTCGATACAATAAACGCCATTTGACCATTCACC
ACATTGGTGTGCACCTCCAAGCTCGAGCTCGTTTA
GTGAACCGTCAGATCGCCTGGAGACGCCATCCACG
CTGTTTTGACCTCCATAGAAGACACCGGGACCGAT
CCAGCCTCCCCTCGAAGCTAGCGATTAGGCATCTC
CTATGGCAGGAAGAAGCGGAGACAGCGACGAAGAA
CTCCTCAAGGCAGTCAGACTCATCAAGTTTCTCTA
TCAAAGCAACCCACCTCCCAATCCCGAGGGGACCC
GACAGGCCCGAAGGAATAGAAGAAGAAGGTGGAGA
GAGAGACAGAGACAGATCCATTCGATTAGTGAACG
GATCCTTAGCACTTATCTGGGACGATCTGCGGAGC
CTGTGCCTCTTCAGCTACCACCGCTTGAGAGACTT
ACTCTTGATTGTAACGAGGATTGTGGAACTTCTGG
GACGCAGGGGGTGGGAAGCCCTCAAATATTGGTGG
AATCTCCTACAATATTGGAGTCAGGAGCTAAAGAA
TAGTCTAGA

The plasmids used in the packaging systems can be modified with similar elements, and the intron sequences can potentially be removed without loss of vector function. For example, the following elements can replace similar elements in the packaging system:

Promoters: Elongation Factor-1 alpha (EF1-alpha) promoter (SEQ ID NO: 47), phosphoglycerate kinase (PGK) promoter (SEQ ID NO: 48), thyroxin binding globulin promoter (SEQ ID NO: 60), and ubiquitin C (UbC) promoter (SEQ ID NO: 49) can replace the CMV promoter (SEQ ID NO: 27) or CMV enhancer/chicken beta actin promoter (SEQ ID NO: 21). These sequences can also be further varied by addition, substitution, deletion or mutation.

Poly A sequences: SV40 poly A (SEQ ID NO: 50) and bGH poly A (SEQ ID NO: 30 or SEQ ID NO: 51) can replace the rabbit beta globin poly A (SEQ ID NO: 40). These sequences can also be further varied by addition, substitution, deletion or mutation.

HIV Gag, Pol, and Integrase sequences: The HIV sequences in the Helper plasmid can be constructed from different HIV strains or clades. For example, HIV Gag (SEQ ID NO: 22); HIV Pol (SEQ ID NO: 23); and HIV Int (SEQ ID NO: 24) from the Bal strain can be interchanged with the gag, pol, and int sequences contained in the helper/helper plus Rev plasmids as outlined herein. These sequences can also be further varied by addition, substitution, deletion or mutation.

Envelope: The VSV-G glycoprotein can be substituted with membrane glycoproteins from feline endogenous virus (RD114) envelope (SEQ ID NO: 52), gibbon ape leukemia virus (GALV) envelope (SEQ ID NO: 53), Rabies (FUG) envelope (SEQ ID NO: 54), lymphocytic choriomeningitis virus (LCMV) envelope (SEQ ID NO: 55), influenza A fowl plague virus (FPV) envelope (SEQ ID NO: 56), Ross River alphavirus (RRV) envelope (SEQ ID NO: 57), murine leukemia virus 10A1 (MLV 10A1) envelope (SEQ ID NO: 58), or Ebola virus (EboV) envelope (SEQ ID NO: 59). Sequences for these envelopes are identified in the sequence portion herein. Further, these sequences can also be further varied by addition, substitution, deletion or mutation.

In summary, the 3-vector versus 4-vector systems can be compared and contrasted as follows. The 3-vector lentiviral vector system may comprise: (1) Helper plasmid: HIV Gag, Pol, Integrase fragment (SEQ ID NO: 43), RRE, and Rev; (2) Envelope plasmid: VSV-G envelope; and (3) Therapeutic vector: RSV, 5′LTR, Psi Packaging Signal, RRE, cPPT, prothrombin enhancer, alpha 1 anti-trypsin promoter, phenylalanine hydroxylase, WPRE, and 3′delta LTR. The 4-vector lentiviral vector system may comprise: (1) Helper plasmid: HIV Gag, Pol, Integrase fragment (SEQ ID NO: 43), and RRE; (2) Rev plasmid: Rev; (3) Envelope plasmid: VSV-G envelope; and (4) Therapeutic vector: RSV, 5′LTR, Psi Packaging Signal, RRE, cPPT, prothrombin enhancer, alpha 1 anti-trypsin promoter, phenylalanine hydroxylase, WPRE, and 3′delta LTR. Sequences corresponding with the above elements are identified in the sequence listings portion herein.

### Example 2. Therapeutic Vectors

Exemplary therapeutic vectors have been designed and developed as shown, for example, in FIG. 3.

Referring first to Vector A of FIG. 3, from left to right, the key genetic elements are as follows: hybrid 5′ long terminal repeat (RSV/LTR), Psi sequence (RNA packaging site), RRE (Rev-response element), cPPT (polypurine tract), a prothrombin enhancer, a hAAT promoter, a PAH sequence including, in embodiments, a codon-optimized PAH sequence or variant thereof, as detailed herein, Woodchuck Post-Transcriptional Regulatory Element (WPRE), and LTR with a deletion in the U3 region.

Referring next to Vector B of FIG. 3, from left to right, the key genetic elements are as follows: hybrid 5′ long terminal repeat (RSV/LTR), Psi sequence (RNA packaging site), RRE (Rev-response element), cPPT (polypurine tract), one HNF1/HNF4 (hepatocyte nuclear factor) binding site upstream of a prothrombin enhancer, a hAAT promoter, a PAH sequence including, in embodiments, a codon-optimized PAH sequence or variant thereof, as detailed herein, a Woodchuck Post-Transcriptional Regulatory Element (WPRE), and LTR with a deletion in the U3 region.

Referring next to Vector C of FIG. 3, from left to right, the key genetic elements are as follows: hybrid 5′ long terminal repeat (RSV/LTR), Psi sequence (RNA packaging site), RRE (Rev-response element), cPPT (polypurine tract), three HNF1/4 (hepatocyte nuclear factor) binding sites upstream of a prothrombin enhancer, a hAAT promoter, a PAH sequence including, in embodiments, a codon-optimized PAH sequence or variant thereof, as detailed herein, a Woodchuck Post-Transcriptional Regulatory Element (WPRE), and LTR with a deletion in the U3 region.

Referring next to Vector D of FIG. 3, from left to right, the key genetic elements are as follows: hybrid 5′ long terminal repeat (RSV/LTR), Psi sequence (RNA packaging site), RRE (Rev-response element), cPPT (polypurine tract), five HNF1 (hepatocyte nuclear factor) binding sites upstream of a prothrombin enhancer, a hAAT promoter, a PAH sequence including, in embodiments, a codon-optimized PAH sequence or variant thereof, as detailed herein, a Woodchuck Post-Transcriptional Regulatory Element (WPRE), and LTR with a deletion in the U3 region.

To produce the vectors outlined generally in FIG. 3, the methods and materials described herein and as otherwise as understood by those skilled in the art were employed.

Inhibitory RNA Design: The sequence of Homo sapiens phenylalanine hydroxylase (PAH) (NM_000277.1) mRNA was used to search for potential shRNA candidates to knockdown PAH levels in human cells. Potential RNA shRNA sequences were chosen from candidates selected by siRNA or shRNA design programs such as from the GPP Web Portal hosted by the Broad Institute (portals.broadinstitute.org/gpp/public/) or the BLOCK-iT RNAi Designer from Thermo Scientific (https://maidesigner.thermofisher.com/maiexpress/). Individual selected shRNA sequences were inserted into a lentiviral vector immediately 3 prime to a RNA polymerase III promoter H1 (H1 Promoter) (SEQ ID NO: 20) to regulate shRNA expression. These lentivirus shRNA constructs were used to transduce cells and measure the change in specific mRNA levels.

Vector Construction: To synthesize shRNA sequences that targeted PAH, oligonucleotide sequences containing BamHI and EcoRI restriction sites were synthesized by Eurofins MWG Operon. Overlapping sense and antisense oligonucleotide sequences were mixed and annealed during cooling from 70 degrees Celsius to room temperature. The lentiviral vector was digested with the restriction enzymes BamHI and EcoRI for one hour at 37 degrees Celsius. The digested lentiviral vector was purified by agarose gel electrophoresis and extracted from the gel using a DNA gel extraction kit from Thermo Scientific. The DNA concentrations were determined and vector to oligo (3:1 ratio) were mixed, allowed to anneal, and ligated. The ligation reaction was performed with T4 DNA ligase for 30 minutes at room temperature. 2.5 microliters of the ligation mix were added to 25 microliters of STBL3 competent bacterial cells. Transformation was achieved after heat-shock at 42 degrees Celsius. Bacterial cells were spread on agar plates containing ampicillin and drug-resistant colonies (indicating the presence of ampicillin-resistance plasmids) were recovered and expanded in LB broth. To check for insertion of the oligo sequences, plasmid DNA was extracted from harvested bacteria cultures with the Thermo Scientific DNA mini prep kit. Insertion of shRNA sequences in the lentiviral vector was verified by DNA sequencing using a specific primer for the promoter used to regulate shRNA expression. Using the following coding sequences, exemplary shRNA sequences were determined to knock-down PAH.

PAH shRNA sequence #1:
(SEQ ID NO: 11)
TCGCATTTCATCAAGATTAATCTCGAG
ATTAATCTTGATGAAATGCGATTTTT
PAH shRNA sequence #2:
(SEQ ID NO: 12)
ACTCATAAAGGAGCATATAAGCTCGAG
CTTATATGCTCCTTTATGAGTTTTTT

### Example 3. Liver Specific Prothrombin Enhancer/hAAT Promoter

Hepa1-6 mouse hepatoma and Hep3B human carcinoma cells were transduced with lentiviral vectors containing a liver-specific prothrombin enhancer (SEQ ID NO: 3), and a human alpha-1 anti-trypsin promoter (SEQ ID NO: 4) to create a DNA fragment containing a prothrombin enhancer and a human alpha-1 anti-trypsin promoter. The resulting DNA sequence is as follows: GCGAGAACTTGTGCCTCCCCGTGTCCTGCTCTTTGTCCCTCTGTCCTACTAGAC TAATATTTTGCCTGGGTACTGCAAACAGGAAATGGGGGAGGGACAGGAGTAGGG CGGAGGGTAGCCCGGGGATCTGCTACCAGTGGAACAGCCACTAAGGATTCTGC AGTGAGAGCAGAGGGCCAGCTAAGTGGTACTCTCCCAGAGACTGTCTGACTCAC GCCACCCCCTCCACCTTGGACACAGGACGCTGTGGCTGAGCCAGGTACAATG ACTCCTTTCGGTAAGTGCAGTGGAAGCTGTACACTGCCCAGGCAAAGCGTCCGG GCAGCGTAGGCGGGCGACTCAGATCCCAGCCAGTGGACTAGCCCCTGTTTGCTC CTCCGATAACTGGGGTGACCTTGGTAATATCACCAGCAGCCTCCCCCGTTGCC CCTCTGGATCCACTGCTAAATACGGACGAGGACAGGGCCCTGTCTCCTCAGCT CAGGCACCACCACTGACCTGGGACAGTGAAT (SEQ ID NO: 61). Results for these infections are detailed in further Examples herein.

### Example 4. hAAT Promoter with Prothrombin Enhancer and Hepatocyte Nuclear Factor (HNF) Binding Sites

Hepa1-6 mouse hepatoma and Hep3B human carcinoma cells were transduced with lentiviral vectors containing a liver-specific prothrombin enhancer (SEQ ID NO: 3), a human alpha-1 anti-trypsin promoter (SEQ ID NO: 4), and one or more hepatocyte nuclear factor (HNF) binding sites. The resulting DNA sequence that includes a DNA fragment containing a prothrombin enhancer, a human alpha-1 anti-trypsin promoter, and five HNF1 binding sites (designated in underlined font) was as follows:

(SEQ ID NO: 62)
GTTAATCATTAACGTTAATCATTAACGTTAATCAT
TAACGTTAATCATTAACGTTAATCATTAACATCGA
TGCGAGAACTTGTGCCTCCCCGTGTTCCTGCTCTT
TGTCCCTCTGTCCTACTTAGACTAATATTTGCCTT
GGGTACTGCAAACAGGAAATGGGGGAGGGACAGGA
GTAGGGCGGAGGGTAGGATTCTGCAGTGAGAGCAG
AGGGCCAGCTAAGTGGTACTCTCCCAGAGACTGTC
TGACTCACGCCACCCCCTCCACCTTGGACACAGGA
CGCTGTGGTTTCTGAGCCAGGTACAATGACTCCTT
TCGGTAAGTGCAGTGGAAGCTGTACACTGCCCAGG
CAAAGCGTCCGGGCAGCGTAGGCGGGCGACTCAGA
TCCCAGCCAGTGGACTTAGCCCCTGTTTGCTCCTC
CGATAACTGGGGTGACCTTGGTTAATATTCACCAG
CAGCCTCCCCCGTTGCCCCTCTGGATCCACTGCTT
AAATACGGACGAGGACAGGGCCCTGTCTCCTCAGC
TTCAGGCACCACCACTGACCTGGGACAGTGAAT.

The resulting DNA sequence that includes a DNA fragment containing a prothrombin enhancer, a human alpha-1 anti-trypsin promoter, and one HNF1/HNF4 binding site (HNF1 designated in underlined font; HNF4 designated in bold font) is as follows:

(SEQ ID NO: 77)
GTTAATCATTAACGCTTGTACTTTGGTACAATCGA
TGCGAGAACTTGTGCCTCCCCGTGTTCCTGCTCTT
TGTCCCTCTGTCCTACTTAGACTAATATTTGCCTT
GGGTACTGCAAACAGGAAATGGGGGAGGGACAGGA
GTAGGGCGGAGGGTAGCCCGGGGATTCTGCAGTGA
GAGCAGAGGGCCAGCTAAGTGGTACTCTCCCAGAG
ACTGTCTGACTCACGCCACCCCCTCCACCTTGGAC
ACAGGACGCTGTGGTTTCTGAGCCAGGTACAATGA
CTCCTTTCGGTAAGTGCAGTGGAAGCTGTACACTG
CCCAGGCAAAGCGTCCGGGCAGCGTAGGCGGGCGA
CTCAGATCCCAGCCAGTGGACTTAGCCCCTGTTTG
CTCCTCCGATAACTGGGGTGACCTTGGTTAATATT
CACCAGCAGCCTCCCCCGTTGCCCCTCTGGATCCA
CTGCTTAAATACGGACGAGGACAGGGCCCTGTCTC
CTCAGCTTCAGGCACCACCACTGACCTGGGACAGT
GAAT.

The resulting DNA sequence that includes a DNA fragment containing a prothrombin enhancer, a human alpha-1 anti-trypsin promoter, and three HNF1/HNF4 binding sites (HNF1 designated in underlined font; HNF4 designated in bold font) is as follows:

(SEQ ID NO: 63)
GTTAATCATTAACGCTTGTACTTTGGTACAGTTAA
TCATTAACGCTTGTACTTTGGTACAGTTAATCATT
AACGCTTGTACTTTGGTACAATCGATGCGAGAACT
TGTGCCTCCCCGTGTTCCTGCTCTTTGTCCCTCTG
TCCTACTTAGACTAATATTTGCCTTGGGTACTGCA
AACAGGAAATGGGGGAGGGACAGGAGTAGGGCGGA
GGGTAGCCCGGGGATTCTGCAGTGAGAGCAGAGGG
CCAGCTAAGTGGTACTCTCCCAGAGACTGTCTGAC
TCACGCCACCCCCTCCACCTTGGACACAGGACGCT
GTGGTTTCTGAGCCAGGTACAATGACTCCTTTCGG
TAAGTGCAGTGGAAGCTGTACACTGCCCAGGCAAA
GCGTCCGGGCAGCGTAGGCGGGCGACTCAGATCCC
AGCCAGTGGACTTAGCCCCTGTTTGCTCCTCCGAT
AACTGGGGTGACCTTGGTTAATATTCACCAGCAGC
CTCCCCCGTTGCCCCTCTGGATCCACTGCTTAAAT
ACGGACGAGGACAGGGCCCTGTCTCCTCAGCTTCA
GGCACCACCACTGACCTGGGACAGTGAAT.

The expression of codon-optimized PAH from these vectors is detailed in further Examples herein.

### Example 5. Materials and Methods for Synthesizing Vectors Containing PAH

The sequence of Homo sapiens phenylalanine hydroxylase (hPAH) miRNA (Gen Bank: NM_000277.1) was chemically synthesized with EcoRI and Sail restriction enzyme sites located at distal and proximal ends of the gene by Eurofins Genomics (Louisville, Ky.). hPAH treated with EcoRI and SalI restriction enzymes was ligated into the pCDH lentiviral plasmids (System Biosciences, CA) under control of a hybrid promoter comprising parts of ApoE (NM_000001.11, U35114.1) or prothrombin (AF478696.1), and hAAT (HG98385.1) locus control regions.

The lentiviral vector and hPAH sequences were digested with the restriction enzymes BamHI and EcoRI (NEB, Ipswich, Mass.) for two hours at 37 degrees Celsius. The digested lentiviral vector was purified by agarose gel electrophoresis and extracted from the gel using a DNA gel extraction kit from ThermoFisher (Waltham, Mass.). The DNA concentration was determined and then mixed with the PAH sequence using an insert to vector ratio of 3:1. The mixture was ligated with T4 DNA ligase (NEB) for 30 minutes at room temperature. 2.5 microliters of the ligation mix were added to 25 microliters of STBL3 competent bacterial cells (ThermoFisher). Transformation was carried out by heat-shock at 42 degrees Celsius. Bacterial cells were streaked onto agar plates containing ampicillin and then colonies were expanded in LB broth. To check for insertion of the PAH sequences, Plasmid DNA was extracted from harvested bacteria cultures with the ThermoFisher DNA mini prep kit. Insertion of the PAH sequence in the lentiviral vector (LV) was verified by DNA sequencing (Eurofins Genomics). Next, the ApoE enhancer/hAAT promoter or prothrombin enhancer/hAAT promoter sequences with ClaI and EcoRI restriction sites were synthesized by Eurofins Genomics. The lentiviral vector containing a PAH coding sequence and the hybrid promoters were digested with ClaI and EcoRI enzymes and ligated together. The plasmids containing the hybrid promoters were verified by DNA sequencing. The lentiviral vector containing hPAH and a hybrid promoter sequence were then used to package lentiviral particles to test for their ability to express PAH in transduced cells. Mammalian cells were transduced with lentiviral particles. Cells were collected after 3 days and protein was analyzed by immunoblot for PAH expression.

Regulation of the hPAH Sequence:

A liver specific enhancer-promoter was added to the lentiviral vector to regulate PAH expression in a liver-specific manner. Specifically, the prothrombin enhancer was combined with the human alpha-1-anti-trypsin promoter in the lentiviral vector to regulate PAH expression. Restricting transgene expression to liver cells is an important consideration for vector safety and target specificity for a genetic medicine to treat phenylketonuria.

### Example 6. Synthesis of Codon-Optimized PAH Sequences

Certain bases within codons were changed in the Homo sapiens phenylalanine hydroxylase (hPAH) mRNA (Gen Bank: NM_000277.1) sequence to create the OPT2 PAH sequence (SEQ ID NO: 2) and OPT3 PAH codon-optimized sequence (SEQ ID NO: 70). The OPT2 and OPT3 PAH sequences flanked with EcoRI and SalI restriction sites were synthesized by Eurofins Genomics and IDT and ligated into a lentiviral vector digested with EcoRI and SalI.

Hybrid PAH codon-optimized sequences were constructed by restriction endonuclease digestion with StuI (New England Biolabs). A C-terminal fragment was digested from the LV-Pro-hAAT-PAH plasmid containing either the OPT2 or OPT3 sequences. The C-terminal OPT3 fragment was ligated back to the plasmid containing the N-terminal OPT2 sequence to create the OPT2/3 sequence (SEQ ID NO: 71). The C-terminal OPT2 sequence was ligated back to the plasmid containing the N-terminal OPT3 sequence to create the OPT3/2 sequence (SEQ ID NO: 72). The correct orientation of the fragments was verified by sequencing (Eurofins Genomics).

### Example 7. Expression of PAH with LV-Pro-hAAT-hPAH Expressing Codon-Optimized Versions of PAH in Hepa1-6 Cells

This Example illustrates the expression of PAH using lentiviral vectors that contain Pro hAAT and codon-optimized versions of PAH.

As described in Example 6, hPAH was codon-optimized (GeneArt Thermo and IDT), synthesized (IDT and Eurofins Genomics), and inserted into a lentiviral vector containing the prothrombin enhancer-hAAT promoter. Insertion of the sequences was verified by DNA sequencing (Eurofins Genomics).

Lentiviral vectors containing hPAH or a codon-optimized hPAH were then used to transduce mouse Hepa1-6 cells (American Type Culture Collection). Cells were transduced with lentiviral particles at a multiplicity of infection (MOI) of 5 and after 3 days protein expression was analyzed by immunoblot for PAH expression. Cells were lysed with a Tris-HCl (pH 7.5) buffer containing 1% NP-40 and protease inhibitor mix (Thermo). The cell lysate was centrifuged at 10000 RPM for 15 minutes and protein concentration was determined with the Protein Assay Reagent (Bio-Rad). Protein lysate was separated on a 4-12% Tris-Bis gel (Thermo) and transferred for 12 hours in a Bio-Rad transfer unit. The expression of PAH was detected by immunoblot using an anti-PAH antibody (MilliporeSigma) and an anti-beta actin antibody (MilliporeSigma) was used for the loading control. PAH expression was driven by a prothrombin enhancer and a hAAT promoter. The lentiviral vectors incorporated, in various instances, either a hPAH or codon-optimized version of the hPAH gene.

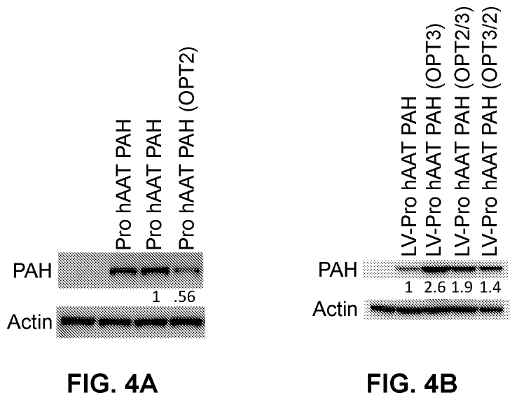

FIG. 4A depicts data demonstrating PAH expression from a lentiviral vector containing prothrombin-hAAT PAH and prothrombin-hAAT codon-optimized PAH (OPT2; SEQ ID NO: 2) in Hepa1-6 cells. The expression of the codon-optimized version of PAH (OPT2) was 44% less than the expression of hPAH. FIG. 4B compares PAH protein expression by immunoblot from a lentiviral vector containing either prothrombin-hAAT PAH or three different codon-optimized versions of PAH in Hepa1-6 cells. The first lane of the immunoblot consists of un-transduced cells, the second lane is cells transduced with a lentivirus expressing the human version of PAH (hPAH) (set at 1), the third lane is cells transduced with a lentivirus expressing codon-optimized version 3 (OPT3; SEQ ID NO: 70) of PAH (2.6 fold increase), the fourth lane is cells transduced with a lentivirus expressing codon-optimized version 2/3 (OPT2/3; SEQ ID NO: 71) of PAH (1.9 fold increase), and the last lane is cells transduced with a lentivirus expressing codon-optimized version 3/2 (OPT3/2; SEQ ID NO: 72) of PAH (1.4 fold increase). The band intensity for each immunoblot was determined by densitometry using Adobe PhotoShop.

As shown in FIGS. 4A and 4B, transduction with the codon-optimized OPT3 PAH sequence resulted in increased PAH expression (i) relative to transduction with the codon-optimized OPT2 (SEQ ID NO: 2), OPT2/3 (SEQ ID NO: 71), and OPT3/2 PAH (SEQ ID NO: 72) sequences and (ii) relative to transduction with the hPAH sequence (SEQ ID NO: 1).

### Example 8. Measuring Expression Levels of PAH mRNA after Transduction of hPAH and Codon-Optimized Versions of PAH in Hepa1-6 Cells

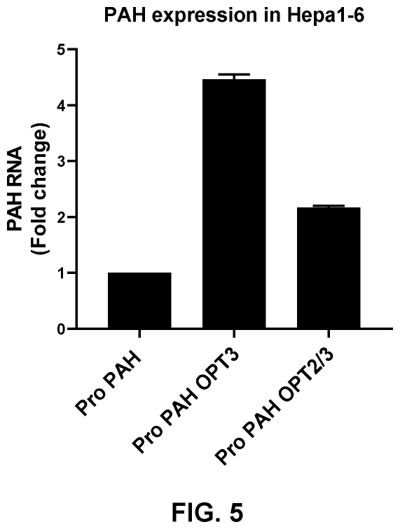

This Example illustrates that expression of PAH RNA is increased in Hepa1-6 carcinoma cells transduced at a MOI of 5 with a lentiviral vector containing prothrombin-hAAT codon-optimized PAH (OPT3 (SEQ ID NO: 70) and OPT2/3 (SEQ ID NO: 71)) relative to a PAH sequence that has not been codon-optimized (SEQ ID NO: 1), as shown in FIG. 5.

hPAH was codon-optimized (GeneArt Thermo), synthesized (IDT and Eurofins Genomics), and inserted into a lentiviral vector containing the prothrombin enhancer-hAAT promoter. Insertion of the sequences was verified by DNA sequencing (Eurofins Genomics). Lentiviral vectors containing non-optimized PAH or codon-optimized PAH were used to transduce Hepa1-6 mouse carcinoma cells (American Type Culture Collection). Cells were transduced with lentiviral particles and after 3 days RNA was extracted with the RNeasy kit (Qiagen) and analyzed by qPCR with a QuantStudio 3 (Thermo). hPAH RNA expression was detected with TaqMan probes and primers (IDT): hPAH FAM TaqMan probe (5′-TCGTGAAAGCTCATGGACAGTGGC-3′: SEQ ID NO: 64) and primer set (PAH TaqMan Forward Primer: 5′-AGATCTTGAGGCATGACATTGG-3′: SEQ ID NO: 65; and PAH TaqMan Reverse Primer: 5′-GTCCAGCTCTTGAATGGTTCT-3′: SEQ ID NO: 66) for hPAH. Total RNA (100 ng) was normalized with an actin FAM probe (5′-AGCGGGAAATCGTGCGTGAC-3′: SEQ ID NO: 67) and primer set (Actin Forward Primer: 5′-GGACCTGACTGACTACCTCAT-3′: SEQ ID NO: 68; and Actin Reverse Primers: 5′-CGTAGCACAGCTTCTCCTTAAT-3′: SEQ ID NO: 69).

As shown in FIG. 5, three groups are compared: Hepa1-6 cells transduced with a lentiviral vector expressing the coding region of PAH (SEQ ID NO: 1) (bar 1) or codon-optimized versions of PAH (OPT3 (SEQ ID NO: 70) and OPT2/3 (SEQ ID NO: 71, bars 2 and 3, respectively) at 5 MOI. PAH RNA levels are expressed as RNA fold change from Hepa1-6 cells expressing PAH (SEQ ID NO: 1) (set at 1). In cells expressing PAH from the codon-optimized version (OPT3: SEQ ID NO: 70), there was a 4.5-fold increase in expression as compared with PAH (SEQ ID NO: 1). In cells expressing PAH from the codon-optimized version (OPT2/3: SEQ ID NO: 71), there was a 2.2-fold increase in expression as compared with PAH (SEQ ID NO: 1).

### Example 9. Lentivirus-Delivered Expression of PAH with a Codon-Optimized PAH Sequence and the Prothrombin Enhancer Containing HNF1 or HNF1/4 Binding Sites in Hepa1-6 and Hep3B Cells

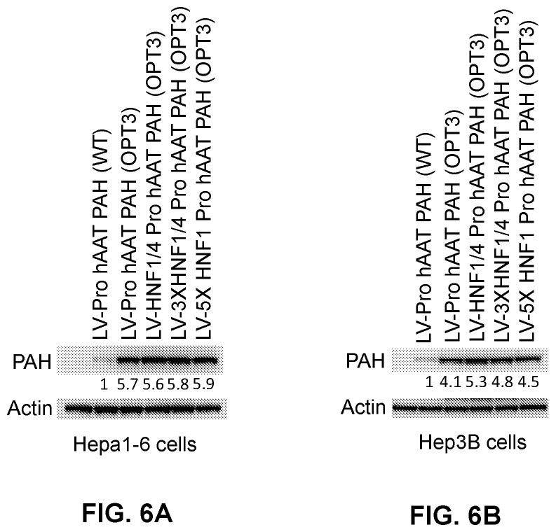

This Example illustrates that expression of codon-optimized hPAH is increased in mouse Hepa1-6 and human Hep3B carcinoma cells when transduced with a lentiviral vector containing the hAAT promoter in combination with the prothrombin enhancer and upstream HNF1/4 binding sites, as shown in FIGS. 6A-6B. This example also shows that a codon-optimized version of the hPAH coding sequence (OPT3) expresses more than the non-optimized hPAH coding region sequence in Hepa1-6 cells and Hep3B cells. This Example further illustrates that a lentiviral vector expressing Hepatocyte Nuclear Factor-1 and -4 (HNF1 and HNF1/4) binding sites in combination with the prothrombin enhancer increases the levels of PAH protein in Hepa1-6 cells and Hep3B cells.

hPAH (optimized and non-optimized) and variations of the prothrombin enhancer with HNF1/4 binding sites were synthesized (Eurofin Genomics and IDT) and inserted into a lentiviral vector containing the hAAT promoter. Insertion of the sequences was verified by DNA sequencing (Eurofin Genomics). The lentiviral vectors containing a verified PAH sequence were then used to transduce Hepa1-6 mouse liver cancer cells (American Type Culture Collection, Manassas). Cells were transduced with lentiviral particles at a MOI of 5 and after 3 days protein were analyzed by immunoblot for PAH expression. Cells were lysed with a Tris-HCl (pH 7.5) buffer containing 1% NP-40 and protease inhibitor mix (Thermo). The cell lysate was centrifuged at 10000 RPM for 15 minutes and protein concentration was determined with the Protein Assay Reagent (Bio-Rad). Protein lysate was separated on a 4-12% Tris-Bis gel (Thermo) and transferred for 12 hours in a Bio-Rad transfer unit. The expression of PAH was detected by immunoblot using an anti-PAH antibody (MilliporeSigma) and an anti-beta actin antibody (MilliporeSigma) was used for the loading control. PAH expression was driven by a prothrombin enhancer and a hAAT promoter. The lentiviral vectors incorporated, in various instances, either codon-optimized versions of the hPAH gene or hPAH genes in which the codons remained unaltered. In addition, PAH expression in these constructs was driven by the hAAT promoter containing the liver-specific prothrombin enhancer with upstream HNF1 or HNF1/4 binding sites. The band intensity for the immunoblots were determined by densitometry using Adobe PhotoShop.

As shown in FIG. 6A, six groups are compared: (1) Hepa1-6 cells alone (lane 1), (2) a lentiviral vector expressing the coding region of hPAH by the prothrombin enhancer/hAAT promoter (lane 2) (Set at 1), (3) a lentiviral vector expressing codon-optimized hPAH (OPT3) by the prothrombin enhancer/hAAT promoter (lane 3) (increase of 5.7-fold), (4) a lentiviral vector expressing codon-optimized hPAH (OPT3) by the prothrombin enhancer/hAAT with one HNF-1 and -4 binding site upstream of the prothrombin enhancer (lane 4) (increase of 5.6-fold), (5) a lentiviral vector expressing codon-optimized hPAH (OPT3) by the prothrombin enhancer/hAAT with three HNF-1 and -4 binding sites upstream of the prothrombin enhancer (lane 5) (increase of 5.8-fold), and (6) a lentiviral vector expressing codon-optimized hPAH (OPT3) by the prothrombin enhancer/hAAT with five HNF-1 binding sites upstream of the prothrombin enhancer (lane 6) (increase of 5.9-fold). The sequence for the hPAH used in this experiment was SEQ ID NO: 1. The sequence used for the codon-optimized PAH used in this experiment was SEQ ID NO: 70.

As shown in FIG. 6B, six groups are compared: (1) Hep3B cells alone (lane 1), (2) a lentiviral vector expressing the coding region of hPAH (SEQ ID NO: 1) by the prothrombin enhancer/hAAT promoter (SEQ ID NO: 61) (lane 2) (set at 1), (3) a lentiviral vector expressing codon-optimized hPAH (OPT3) (SEQ ID NO: 70) by the prothrombin enhancer/hAAT promoter (SEQ ID NO: 61) (lane 3) (increase of 4.1-fold), (4) a lentiviral vector expressing codon-optimized hPAH (OPT3) by the prothrombin enhancer/hAAT promoter with one HNF-1 and -4 binding site (SEQ ID NO: 9) upstream of the prothrombin enhancer (lane 4) (increase of 5.3-fold), (5) a lentiviral vector expressing codon-optimized hPAH (OPT3) by the prothrombin enhancer/hAAT promoter with three HNF-1 and -4 binding sites (SEQ ID NO: 10) upstream of the prothrombin enhancer (lane 5) (increase of 4.8-fold), and (6) a lentiviral vector expressing codon-optimized hPAH (OPT3) by the prothrombin enhancer/hAAT promoter with five HNF-1 binding sites (SEQ ID NO: 8) upstream of the prothrombin enhancer (lane 6) (increase of 4.5-fold).

FIGS. 6A and 6B demonstrate that expression of PAH is increased in Hepa1-6 and Hep3B carcinoma cells when transduced by lentiviral vectors containing a codon-optimized version of PAH (OPT3) that have HNF1 or HNF1/4 binding sites upstream of the prothrombin enhancer versus Hepa1-6 and Hep3B carcinoma cells transduced with PAH.

### Example 10. Lentivirus-Delivered Expression of hPAH in Huh-7 Cells with a Codon-Optimized PAH Sequence and a Regulatory Sequence Containing Either a hAAT Enhancer/Transthyretin Promoter/Minute Virus of Mouse Intron or a Prothrombin Enhancer/hAAT Promoter/Minute Virus of Mouse Intron

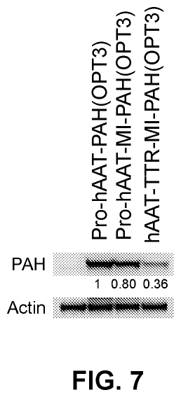

This Example illustrates that expression of codon-optimized human PAH is increased in human hepatocellular carcinoma cells with a lentiviral vector containing liver-specific regulatory elements in comparison to alternative constructs containing introns and alternative enhancer/promoter combinations, as shown in FIG. 7.

The hAAT promoter in combination with the prothrombin enhancer (SEQ ID NO: 61) increased PAH expression, but the addition of an intron sequence from the Minute Virus of Mouse (SEQ ID NO: 80) did not enhance expression. The combination of a prothrombin enhancer and hAAT promoter (SEQ ID NO: 61) with a codon-optimized PAH sequence (SEQ ID NO: 70) resulted in higher expression of PAH as compared with a hAAT promoter (SEQ ID NO: 82) and transthyretin enhancer (SEQ ID NO: 81).

The liver-specific regulatory sequences were synthesized (IDT) and inserted into a lentiviral vector upstream of the optimized PAH sequence. Insertion of the sequences was verified by DNA sequencing (Eurofin Genomics). The lentiviral vectors containing verified sequences were then used to transduce Huh-7 hepatocellular cancer cells (Japanese Collection of Research Bioresources Cell Bank). Cells were transduced with lentiviral particles at a MOI of 50 and after 3 days protein was analyzed by immunoblot for PAH expression. Cells were lysed with a Tris-HCl (pH 7.5) buffer containing 1% NP-40 and protease inhibitor mix (Thermo). The cell lysate was centrifuged at 12,000 RPM for 15 minutes and the protein concentration was determined with the Protein Assay Reagent (Bio-Rad). Protein lysate was separated on a 4-12% Tris-Bis gel (Thermo) and transferred for 16 hours in a Bio-Rad transfer unit. The expression of PAH was detected by immunoblot using an anti-PAH antibody (MilliporeSigma) and an anti-beta actin antibody (MilliporeSigma) was used for the loading control. The band intensity for the immunoblots was determined by densitometry using Adobe PhotoShop.

As shown in FIG. 7, four groups are compared: (i) Huh-7 cells alone (lane 1); (ii) a lentiviral vector expressing codon-optimized hPAH (OPT3; SEQ ID NO: 70) and the prothrombin enhancer/hAAT promoter (SEQ ID NO: 61) (lane 2) (baseline band intensity set at 1); (iii) a lentiviral vector expressing codon-optimized hPAH (OPT3) by a prothrombin enhancer/hAAT promoter and intron sequence of the Minute Virus of Mouse (SEQ ID NO: 78) (lane 3) (band intensity of 0.80); and (iv) a lentiviral vector expressing codon-optimized hPAH (OPT3) by a hAAT promoter/transthyretin enhancer and intron sequence of the Minute Virus of Mouse (SEQ ID NO: 79) (lane 4) (band intensity of 0.36).

The results illustrate that lentiviral vectors encoding an intron sequence from the Minute Virus of Mouse resulted in lower PAH expression relative to lentiviral vectors that lacked this intron sequence (compare lane 2 with lane 3, of FIG. 7). This finding is unexpected because previous research suggests that the intron sequence from the Minute Virus of Mouse increases exogenous gene expression from vectors. In addition, this example unexpectedly shows that lentiviral vectors containing promoter/enhancer combinations used for liver-specific gene expression, resulted in lower PAH expression than lentiviral vectors containing the specific combination of Prothrombin enhancer/hAAT promoter with no additional intron as provided herein (compare lane 2 with lane 4, of FIG. 7).

### Example 11. Lentivirus-Delivered Expression of hPAH in Huh-7 Cells with a Codon-Optimized PAH Sequence with Either a Mutant WPRE Sequence or Short WPRE (WPREs) Sequence and Containing Either a PAH or Albumin 3′ UTR Sequence

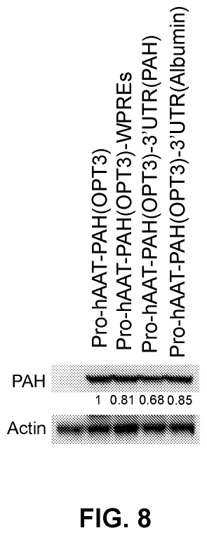

This Example illustrates that expression of codon-optimized human PAH is increased in human hepatocellular carcinoma cells with a lentiviral vector containing liver-specific regulatory elements in comparison to alternative vector constructs comprising 3′UTRs and alternative WPRE sequences, as shown in FIG. 8.

When the WPRE was modified to a shorter, mutant version without the X-protein sequence (SEQ ID NO: 87), the expression of PAH was less than but similar to the vector containing the wild-type WPRE (SEQ ID NO: 18). When a 3′ UTR sequence from either the PAH gene (SEQ ID NO: 85) or albumin gene (SEQ ID NO: 86) was added downstream of the PAH coding sequence, which resulted in either the PAH optimized version 3-PAH 3′UTR sequence (SEQ ID NO: 83) or the PAH optimized version 3-Albumin 3′UTR sequence (SEQ ID NO: 84), there was decreased expression of PAH relative to the vector that did not contain a 3′UTR.

The WPREs and 3′ UTR sequences were synthesized (IDT) and inserted into a lentiviral vector upstream of the optimized PAH sequence. Insertion of the sequences was verified by DNA sequencing (Eurofin Genomics). The lentiviral vectors containing verified sequences were then used to transduce Huh-7 hepatocellular cancer cells (Japanese Collection of Research Bioresources Cell Bank). Cells were transduced with lentiviral particles at a MOI of 50 and after 3 days protein was analyzed by immunoblot for PAH expression. Cells were lysed with a Tris-HCl (pH 7.5) buffer containing 1% NP-40 and protease inhibitor mix (Thermo). The cell lysate was centrifuged at 12,000 RPM for 15 minutes and the protein concentration was determined with the Protein Assay Reagent (Bio-Rad). Protein lysate was separated on a 4-12% Tris-Bis gel (Thermo) and transferred for 16 hours in a Bio-Rad transfer unit. The expression of PAH was detected by immunoblot using an anti-PAH antibody (MilliporeSigma) and an anti-beta actin antibody (MilliporeSigma) was used for the loading control. The band intensity for the immunoblots was determined by densitometry using Adobe PhotoShop.

As shown in FIG. 8, five groups are compared: (i) Huh-7 cells alone (lane 1); (ii) a lentiviral vector expressing codon-optimized hPAH (OPT3; SEQ ID NO: 70), a prothrombin enhancer/hAAT promoter (SEQ ID NO: 61), and a wild-type WPRE (SEQ ID NO: 18) (lane 2) (baseline band intensity set at 1); (iii) a lentiviral vector expressing codon-optimized hPAH (OPT3; SEQ ID NO: 70), a prothrombin enhancer/hAAT promoter (SEQ ID NO: 61), and a mutant WPRE lacking expression of the X-protein (SEQ ID NO: 87) (lane 3) (band intensity of 0.81); (iv) a lentiviral vector expressing codon-optimized hPAH (OPT3; SEQ ID NO: 70), a prothrombin enhancer/hAAT promoter (SEQ ID NO: 61), and with a PAH 3′ UTR (SEQ ID NO: 85) (lane 4) (band intensity of 0.68); and (v) a lentiviral vector expressing codon-optimized hPAH (OPT3; SEQ ID NO: 70) and a prothrombin enhancer/hAAT promoter (SEQ ID NO: 61) and with a albumin 3′ UTR (SEQ ID NO: 86) (lane 5) (band intensity of 0.85).

The results illustrate that lentiviral vectors substituting a mutant WPRE for the normally used wild-type WPRE, or adding the natural 3′ UTR of human PAH gene, or adding a 3′ UTR from the human albumin gene, that are then used for cell transduction, results in lower expression of PAH compared to the Pro-hAAT-PAH(OPT3) vector containing wild-type WPRE and no 3′ UTR sequence. The results also illustrate the negative effect on PAH expression using a lentiviral vector that encodes natural human PAH 3′UTR relative to a lentiviral vector that encodes an albumin PAH 3′UTR (compare lane 4 with lane 5, of FIG. 8). This finding may be due to a change in secondary structure of the PAH mRNA that results when using the albumin PAH 3′UTR versus the natural human PAH 3′UTR. This change in secondary structure may be reducing the interactions between the coding region of PAH and the 3′UTR, thereby resulting in higher PAH expression levels. Moreover, as shown in this example, when a lentiviral vector is used that lacks a 3′UTR PAH, expression levels of PAH are the highest (compare lanes 4 and 5 with lane 2, of FIG. 8).

Sequence Listing

| | | |
|---|---|---|
| SEQ | | |
| ID | | |
| NO: | Description | Sequence |

| | | |
|---|---|---|
| 1 | hPAH | ATGTCCACTGCGGTC |
| | | CTGGAAAACCCAGGC |
| | | TTGGGCAGGAAACTC |
| | | TCTGACTTTGGACAG |
| | | GAAACAAGCTATATT |
| | | GAAGACAACTGCAAT |
| | | CAAAATGGTGCCATA |
| | | TCACTGATCTTCTCA |
| | | CTCAAAGAAGAAGTT |
| | | GGTGCATTGGCCAAA |
| | | GTATTGCGCTTATTT |
| | | GAGGAGAATGATGTA |
| | | AACCTGACCCACATT |
| | | GAATCTAGACCTTCT |
| | | CGTTTAAAGAAAGAT |
| | | GAGTATGAATTTTTC |
| | | ACCCATTTGGATAAA |
| | | CGTAGCCTGCCTGCT |
| | | CTGACAAACATCATC |
| | | AAGATCTTGAGGCAT |
| | | GACATTGGTGCCACT |
| | | GTCCATGAGCTTTCA |
| | | CGAGATAAGAAGAAA |
| | | GACACAGTGCCCTGG |
| | | TTCCCAAGAACCATT |
| | | CAAGAGCTGGACAGA |
| | | TTTGCCAATCAGATT |
| | | CTCAGCTATGGAGCG |
| | | GAACTGGATGCTGAC |
| | | CACCCTGGTTTTAAA |
| | | GATCCTGTGTACCGT |
| | | GCAAGACGGAAGCAG |
| | | TTTGCTGACATTGCC |
| | | TACAACTACCGCCAT |
| | | GGGCAGCCCATCCCT |
| | | CGAGTGGAATACATG |
| | | GAGGAAGAAAAGAAA |
| | | ACATGGGGCACAGTG |
| | | TTCAAGACTCTGAAG |
| | | TCCTTGTATAAAACC |
| | | CATGCTTGCTATGAG |
| | | TACAATCACATTTTT |
| | | CCACTTCTTGAAAAG |
| | | TACTGTGGCTTCCAT |
| | | GAAGATAACATTCCC |
| | | CAGCTGGAAGACGTT |
| | | TCTCAATTCCTGCAG |
| | | ACTTGCACTGGTTTC |
| | | CGCCTCCGACCTGTG |
| | | GCTGGCCTGCTTTCC |
| | | TCTCGGGATTTCTTG |
| | | GGTGGCCTGGCCTTC |
| | | CGAGTCTTCCACTGC |
| | | ACACAGTACATCAGA |
| | | CATGGATCCAAGCCC |
| | | ATGTATACCCCCGAA |
| | | CCTGACATCTGCCAT |
| | | GAGCTGTTGGGACAT |
| | | GTGCCCTTGTTTTCA |
| | | GATCGCAGCTTTGCC |
| | | CAGTTTTCCCAGGAA |
| | | ATTGGCCTTGCCTCT |
| | | CTGGGTGCACCTGAT |
| | | GAATACATTGAAAAG |
| | | CTCGCCACAATTTAC |
| | | TGGTTTACTGTGGAG |
| | | TTTGGGCTCTGCAAA |
| | | CAAGGAGACTCCATA |
| | | AAGGCATATGGTGCT |
| | | GGGCTCCTGTCATCC |
| | | TTTGGTGAATTACAG |
| | | TACTGCTTATCAGAG |
| | | AAGCCAAAGCTTCTC |
| | | CCCCTGGAGCTGGAG |
| | | AAGACAGCCATCCAA |
| | | AATTACACTGTCACG |
| | | GAGTTCCAGCCCCTG |
| | | TATTACGTGGCAGAG |
| | | AGTTTTAATGATGCC |
| | | AAGGAGAAAGTAAGG |
| | | AACTTTGCTGCCACA |
| | | ATACCTCGGCCCTTC |
| | | TCAGTTCGCTACGAC |
| | | CCATACACCCAAAGG |
| | | ATTGAGGTCTTGGAC |
| | | AATACCCAGCAGCTT |
| | | AAGATTTTGGCTGAT |
| | | TCCATTAACAGTGAA |
| | | ATTGGAATCCTTTGC |
| | | AGTGCCCTCCAGAAA |
| | | ATAAAGTAA |
| 2 | Codon- | ATGAGTACGGCTGTG |
| | optimized | CTCGAGAATCCAGGT |
| | PAH (Opt2) | TTGGGCCGAAAGCTG |
| | | TCTGATTTTGGACAG |
| | | GAGACATCTTATATT |
| | | GAAGACAACTGCAAC |
| | | CAGAATGGTGCGATA |
| | | TCCCTTATTTTTTCT |
| | | CTGAAAGAAGAAGTA |
| | | GGTGCGCTGGCAAAG |
| | | GTCTTGCGGCTGTTT |
| | | GAAGAGAACGATGTT |
| | | AATCTTACTCATATT |
| | | GAGTCCAGACCATCA |
| | | CGGCTGAAAAAAGAC |
| | | GAGTACGAATTTTTT |
| | | ACTCACTTGGACAAA |
| | | CGAAGCTTGCCGGCT |
| | | CTTACTAATATCATT |
| | | AAGATCCTCCGGCAT |
| | | GACATAGGGGCGACA |
| | | GTGCATGAGCTTTCA |
| | | AGGGATAAAAAGAAA |
| | | GATACCGTCCCCTGG |
| | | TTTCCAAGGACCATA |
| | | CAAGAACTCGACCGA |
| | | TTCGCGAACCAGATC |
| | | CTTTCATATGGTGCT |
| | | GAGTTGGATGCTGAC |
| | | CACCCCGGCTTCAAA |
| | | GACCCGGTCTACCGA |
| | | GCGCGGCGGAAACAA |
| | | TTTGCTGACATCGCA |
| | | TACAATTACAGGCAT |
| | | GGCCAGCCAATTCCT |
| | | AGAGTAGAATACATG |
| | | GAAGAAGAGAAAAAA |
| | | ACCTGGGGTACCGTC |
| | | TTCAAGACGCTGAAA |
| | | TCATTGTATAAAACT |
| | | CATGCATGTTACGAA |
| | | TATAACCATATTTTT |
| | | CCGTTGCTCGAGAAA |
| | | TATTGCGGGTTCCAC |
| | | GAAGATAACATCCCA |
| | | CAACTCGAGGATGTA |
| | | TCTCAGTTCCTCCAG |
| | | ACCTGTACGGGGTTT |
| | | CGACTTAGGCCTGTC |
| | | GCGGGTTTGCTCAGT |
| | | TCTCGAGACTTCCTG |
| | | GGTGGATTGGCGTTT |
| | | CGGGTATTCCATTGC |
| | | ACGCAGTATATCCGA |
| | | CACGGAAGTAAGCCA |
| | | ATGTACACGCCAGA |
| | | ACCCGATATCTGTCA |
| | | CGAATTGCTTGGACA |
| | | CGTTCCTCTGTTTTC |
| | | TGATCGATCATTCGC |
| | | TCAGTTTTCACAGGA |
| | | AATCGGCCTGGCATC |
| | | TTTGGGAGCGCCGGA |
| | | TGAATATATTGAGAA |
| | | GCTCGCTACAATTTA |
| | | CTGGTTCACGGTAGA |
| | | ATTTGGGTTGTGCAA |
| | | GCAGGGTGATAGTAT |
| | | TAAAGCATACGGTGC |
| | | GGGATTGCTGTCCTC |
| | | ATTCGGGGAGCTTCA |
| | | GTATTGCCTGTCCGA |
| | | GAAACCCAAGCTGTT |
| | | GCCGTTGGAATTGGA |
| | | AAAAACCGCTATCCA |
| | | AAATTACACAGTAAC |
| | | GGAGTTCCAACCTTT |
| | | GTACTACGTAGCCGA |
| | | GTCATTTAACGATGC |
| | | AAAGGAGAAGGTCAG |
| | | AAATTTTGCTGCGAC |
| | | GATACCCAGACCGTT |
| | | CTCAGTAAGGTACGA |
| | | TCCTTACACTCAGAG |
| | | GATTGAAGTCCTGGA |
| | | TAATACGCAACAGCT |
| | | CAAGATCCTGGCAGA |
| | | CTCCATAAATTCTGA |
| | | AATCGGCATCTTGTG |
| | | TTCAGCACTGCAAAA |
| | | GATAAAATAA |
| 3 | Prothrombin | GCGAGAACTTGTGCC |
| | enhancer(Pro) | TCCCCGTGTTCCTGC |
| | | TCTTTGTCCCTCTGT |
| | | CCTACTTAGACTAAT |
| | | ATTTGCCTTGGGTAC |
| | | TGCAAACAGGAAATG |
| | | GGGGAGGGACAGGAG |
| | | TAGGGCGGAGGGTAG |
| 4 | Human alpha- | GATCTTGCTACCAGT |
| | 1 anti-trypsin | GGAACAGCCACTAAG |
| | promoter | GATTCTGCAGTGAGA |
| | (hAAT) | GCAGAGGGCCAGCTA |
| | | AGTGGTACTCTCCCA |
| | | GAGACTGTCTGACTC |
| | | ACGCCACCCCCTCCA |
| | | CCTTGGACACAGGAC |
| | | GCTGTGGTTTCTGAG |
| | | CCAGGTACAATGACT |
| | | CCTTTCGGTAAGTGC |
| | | AGTGGAAGCTGTACA |
| | | CTGCCCAGGCAAAGC |
| | | GTCCGGGCAGCGTAG |
| | | GCGGGCGACTCAGAT |
| | | CCCAGCCAGTGGACT |
| | | TAGCCCCTGTTTGCT |
| | | CCTCCGATAACTGGG |
| | | GTGACCTTGGTTAAT |
| | | ATTCACCAGCAGCCT |
| | | CCCCCGTTGCCCCTC |
| | | TGGATCCACTGCTTA |
| | | AATACGGACGAGGAC |
| | | AGGGCCCTGTCTCCT |
| | | CAGCTTCAGGCACCA |
| | | CCACTGACCTGGGAC |
| | | AGTGAAT |
| 5 | Rabbit beta | GTGAGTTTGGGGACC |
| | globin intron | CTTGATTGTTCTTTC |
| | | TTTTTCGCTATTGTA |
| | | AAATTCATGTTATAT |
| | | GGAGGGGGCAAAGTT |
| | | TTCAGGGTGTTGTTT |
| | | AGAATGGGAAGATGT |
| | | CCCTTGTATCACCAT |
| | | GGACCCTCATGATAA |
| | | TTTTGTTTCTTTCAC |
| | | TTTCTACTCTGTTGA |
| | | CAACCATTGTCTCCT |
| | | CTTATTTTCTTTTCA |
| | | TTTTCTGTAACTTTT |
| | | TCGTTAAACTTTAGC |
| | | TTGCATTTGTAACGA |
| | | ATTTTTAAATTCACT |
| | | TTTGTTTATTTGTCA |
| | | GATTGTAAGTACTTT |
| | | CTAGCACAGTTTTAG |
| | | AGAACAATTGTTATA |
| | | ATTAAATGATAAGGT |
| | | AGAATATTTCTGCAT |
| | | ATAAATTCTGGCTGG |
| | | CGTGGAAATATTCTT |
| | | ATTGGTAGAAACAAC |
| | | TACACCCTGGTCATC |
| | | ATCCTGCCTTTCTCT |
| | | TTATGGTTACAATGA |
| | | TATACACTGTTTGAG |
| | | ATGAGGATAAAATAC |
| | | TCTGAGTCCAAACCG |
| | | GGCCCCTCTGCTAAC |
| | | CATGTTCATGCCTTC |
| | | TTCTCTTTCCTACAG |
| 6 | Human beta | GGATCCTGAGAACTT |
| | globin intron | CAGGGTGAGTCTATG |
| | | GGACGCTTGATGTTT |
| | | TCTTTCCCCTTCTTT |
| | | TCTATGGTTAAGTTC |
| | | ATGTCATAGGAAGGG |
| | | GATAAGTAACAGGGT |
| | | ACACATATTGACCAA |
| | | ATCAGGGTAATTTTG |
| | | CATTTGTAATTTTAA |
| | | AAAATGCTTTCTTCT |
| | | TTTAATATACTTTTT |
| | | TGTTTATCTTATTTC |
| | | TAATACTTTCCCTAA |
| | | TCTCTTTCTTTCAGG |
| | | GCAATAATGATACAA |
| | | TGTATCATGCCTCTT |
| | | TGCACCATTCTAAAG |
| | | AATAACAGTGATAAT |
| | | TTCTGGGTTAAGGCA |
| | | ATAGCAATATTTCTG |
| | | CATATAAATATTTCT |
| | | GCATATAAATTGTAA |
| | | CTGATGTAAGAGGTT |
| | | TCATATTGCTAATAG |
| | | CAGCTACAATCCAGC |
| | | TACCATTCTGCTTTT |
| | | ATTTTATGGTTGGGA |
| | | TAAGGCTGGATTATT |
| | | CTGAGTCCAAGCTAG |
| | | GCCCTTTTGCTAATC |
| | | ATGTTCATACCTCTT |
| | | ATCTTCCTCCCACAG |
| | | CTCCTGGGCAACGTG |
| | | CTGGTCTGTGTGCTG |
| | | GCCCATCACTTTGGC |
| | | AAAG |
| 7 | IX | GTTAATCATTAAC |
| | Hepatocyte | |
| | Nuclear Factor | |
| | 1 (1XHNFI) | |
| 8 | 5XHcpatocyte | GTTAATCATTAACGT |
| | Nuclear Factor | TAATCATTAACGTTA |
| | 1 (5XHNFI) | ATCATTAACGTTAAT |
| | | CATTAACGTTAATCA |
| | | TTAAC |
| 9 | IXHepatocvtc | GTTAATCATTAACGC |
| | Nuclear Factor | TTGTACTTTGGTACA |
| | 1/4(IXHNF1/4) | |
| 10 | 3XHepatocvtc | GTTAATCATTAACGC |
| | Nuclear Factor | TTGTACTTTGGTACA |
| | 1/4(3XHNF1/4) | GTTAATCATTAACGC |
| | | TTGTACTTTGGTACA |
| | | GTTAATCATTAACGC |
| | | TTGTACTTTGGTACA |
| 11 | PAH shRNA | TCGCATTTCATCAAG |
| | sequence #1 | ATTAATCTCGAGATT |
| | | AATCTTGATGAAATG |
| | | CGATTTTT |
| 12 | PAH shRNA | ACTCATAAAGGAGCA |
| | sequence #2 | TATAAGCTCGAGCTT |
| | | ATATGCTCCTTTATG |
| | | AGTTTTTT |
| 13 | Rous Sarcoma | GTAGTCTTATGCAAT |
| | virus (RSV) | ACTCTTGTAGTCTTG |
| | promoter | CAACATGGTAACGAT |
| | | GAGTTAGCAACATGC |
| | | CTTACAAGGAGAGAA |
| | | AAAGCACCGTGCATG |
| | | CCGATTGGTGGAAGT |
| | | AAGGTGGTACGATCG |
| | | TGCCTTATTAGGAAG |
| | | GCAACAGACGGGTCT |
| | | GACATGGATTGGACG |
| | | AACCACTGAATTGCC |
| | | GCATTGCAGAGATAT |
| | | TGTATTTAAGTGCCT |
| | | AGCTCGATACAATAA |
| | | ACG |
| 14 | 5′ Long | GGTCTCTCTGGTTAG |
| | terminal | ACCAGATCTGAGCCT |
| | repeal (LTR) | GGGAGCTCTCTGGCT |
| | | AACTAGGGAACCCAC |
| | | TGCTTAAGCCTCAAT |
| | | AAAGCTTGCCTTGAG |
| | | TGCTTCAAGTAGTGT |
| | | GTGCCCGTCTGTTGT |
| | | GTGACTCTGGTAACT |
| | | AGAGATCCCTCAGAC |
| | | CCTTTTAGTCAGTGT |
| | | GGAAAATCTCTAGCA |
| 15 | Psi Packaging | TACGCCAAAAATTTT |
| | signal (RNA | GACTAGCGGAGGCTA |
| | packaging | GAAGGAGAGAG |
| | site) | |
| 16 | Rev response | AGGAGCTTTGTTCCT |
| | element(RRE) | TGGGTTCTTGGGAGC |
| | | AGCAGGAAGCACTAT |
| | | GGGCGCAGCCTCAAT |
| | | GACGCTGACGGTACA |
| | | GGCCAGACAATTATT |
| | | GTCTGGTATAGTGCA |
| | | GCAGCAGAACAATTT |
| | | GCTGAGGGCTATTGA |
| | | GGCGCAACAGCATCT |
| | | GTTGCAACTCACAGT |
| | | CTGGGGCATCAAGCA |
| | | GCTCCAGGCAAGAAT |
| | | CCTGGCTGTGGAAAG |
| | | ATACCTAAAGGATCA |
| | | ACAGCTCC |
| 17 | Central | TTTTAAAAGAAAAGG |
| | poly purine | GGGGATTGGGGGGTA |
| | tract (cPPT) | CAGTGCAGGGGAAAG |
| | (poly purine | AATAGTAGACATAAT |
| | tract) | AGCAACAGACATACA |
| | | AACTAAAGAATTACA |
| | | AAAACAAATTACAAA |
| | | ATTCAAAATTTTA |
| 18 | Long WPRE | AATCAACCTCTGGAT |
| | sequence | TACAAAATTTGTGAA |
| | | AGATTGACTGGTATT |
| | | CTTAACTATGTTGCT |
| | | CCTTTTACGCTATGT |
| | | GGATACGCTGCTTTA |
| | | ATGCCTTTGTATCAT |
| | | GCTATTGCTTCCCGT |
| | | ATGGCTTTCATTTTC |
| | | TCCTCCTTGTATAAA |
| | | TCCTGGTTGCTGTCT |
| | | CTTTATGAGGAGTTG |
| | | TGGCCCGTTGTCAGG |
| | | CAACGTGGCGTGGTG |
| | | TGCACTGTGTTTGCT |
| | | GACGCAACCCCCACT |
| | | GGTTGGGGCATTGCC |
| | | ACCACCTGTCAGCTC |
| | | CTTTCCGGGACTTTC |
| | | GCTTTCCCCCTCCCT |
| | | ATTGCCACGGCGGAA |
| | | CTCATCGCCGCCTGC |
| | | CTTGCCCGCTGCTGG |
| | | ACAGGGGCTCGGCTG |
| | | TTGGGCACTGACAAT |
| | | TCCGTGGTGTTGTCG |
| | | GGGAAATCATCGTCC |
| | | TTTCCTTGGCTGCTC |
| | | GCCTGTGTTGCCACC |
| | | TGGATTCTGCGCGGG |
| | | ACGTCCTTCTGCTAC |
| | | GTCCCTTCGGCCCTC |
| | | AATCCAGCGGACCTT |
| | | CCTTCCCGCGGCCTG |
| | | CTGCCGGCTCTGC |
| | | GGCCTCTTCCGCGTC |
| | | TTCGCCTTCGCCCTC |
| | | AGACGAGTCGGATCT |
| | | CCCTTTGGGCCGCCT |
| | | CCCCGCCTG |
| 19 | delta U3 | TGGAAGGGCTAATTC |
| | 3′LTR | ACTCCCAACGAAGAT |
| | | AAGATCTGCTTTTTG |
| | | CTTGTACTGGGTCTC |
| | | TCTGGTTAGACCAGA |
| | | TCTGAGCCTGGGAGC |
| | | TCTCTGGCTAACTAG |
| | | GGAACCCACTGCTTA |
| | | AGCCTCAATAAAGCT |
| | | TGCCTTGAGTGCTTC |
| | | AAGTAGTGTGTGCCC |
| | | GTCTGTTGTGTGACT |
| | | CTGGTAACTAGAGAT |
| | | CCCTCAGACCCTTTT |
| | | AGTCAGTGTGGAAAA |
| | | TCTCTAGCAGTAGTA |
| | | GTTCATGTCA |
| 20 | H1 Promoter | GAACGCTGACGTCAT |
| | | CAACCCGCTCCAAGG |
| | | AATCGCGGGCCCAGT |
| | | GTCACTAGGCGGGAA |
| | | CACCCAGCGCGCGTG |
| | | CGCCCTGGCAGGAAG |
| | | ATGGCTGTGAGGGAC |
| | | AGGGGAGTGGCGCCC |
| | | TGCAATATTTGCATG |
| | | TCGCTATGTGTTCTG |
| | | GGAAATCACCATAAA |
| | | CGTGAAATGTCTTTG |
| | | GATTTGGGAATCTTA |
| | | TAAGTTCTGTATGAG |
| | | ACCACTT |
| 21 | CMV | TAGTTATTAATAGTA |
| | enhancer/ | ATCAATTACGGGGTC |
| | chicken beta | ATTAGTTCATAGCCC |
| | actin | ATATATGGAGTTCCG |
| | promoter | CGTTACATAACTTAC |
| | | GGTAAATGGCCCGCC |
| | | TGGCTGACCGCCCAA |
| | | CGACCCCCGCCCATT |
| | | GACGTCAATAATGAC |
| | | GTATGTTCCCATAGT |
| | | AACGCCAATAGGGAC |
| | | TTTCCATTGACGTCA |
| | | ATGGGTGGACTATTT |
| | | ACGGTAAACTGCCCA |
| | | CTTGGCAGTACATCA |
| | | AGTGTATCATATGCC |
| | | AAGTACGCCCCCTAT |
| | | TGACGTCAATGACGG |
| | | TAAATGGCCCGCCTG |
| | | GCATTATGCCCAGTA |
| | | CATGACCTTATGGGA |
| | | CTTTCCTACTTGGCA |
| | | GTACATCTACGTATT |
| | | AGTCATCGCTATTAC |
| | | CATGGGTCGAGGTGA |
| | | GCCCCACGTTCTGCT |
| | | TCACTCTCCCCATCT |
| | | CCCCCCCCTCCCCAC |
| | | CCCCAATTTTGTATT |
| | | TATTTATTTTTTAAT |
| | | TATTTTGTGCAGCGA |
| | | TGGGGGCGGGGGGGG |
| | | GGGGGGCGCGCGCCA |
| | | GGCGGGGCGGGGCGG |
| | | GGCGAGGGGCGGGGC |
| | | GGGGCGAGGCGGAGA |
| | | GGTGCGGCGGCAGCC |
| | | AATCAGAGCGGCGCG |
| | | CTCCGAAAGTTTCCT |
| | | TTTATGGCGAGGCGG |
| | | CGGCGGCGGCGGCCC |
| | | TATAAAAAGCGAAGC |
| | | GCGCGGCGGGCG |
| 22 | HIV Gag | ATGGGTGCGAGAGCG |
| | | TCAGTATTAAGCGGG |
| | | GGAGAATTAGATCGA |
| | | TGGGAAAAAATTCGG |
| | | TTAAGGCCAGGGGGA |
| | | AAGAAAAAATATAAA |
| | | TTAAAACATATAGTA |
| | | TGGGCAAGCAGGGAG |
| | | CTAGAACGATTCGCA |
| | | GTTAATCCTGGCCTG |
| | | TTAGAAACATCAGAA |
| | | GGCTGTAGACAAATA |
| | | CTGGGACAGCTACAA |
| | | CCATCCCTTCAGACA |
| | | GGATCAGAAGAACTT |
| | | AGATCATTATATAAT |
| | | ACAGTAGCAACCCTC |
| | | TATTGTGTGCATCAA |
| | | AGGATAGAGATAAAA |
| | | GACACCAAGGAAGCT |
| | | TTAGACAAGATAGAG |
| | | GAAGAGCAAAACAAA |
| | | AGTAAGAAAAAAGCA |
| | | CAGCAAGCAGCAGCT |
| | | GACACAGGACACAGC |
| | | AATCAGGTCAGCCAA |
| | | AATTACCCTATAGTG |
| | | CAGAACATCCAGGGG |
| | | CAAATGGTACATCAG |
| | | GCCATATCACCTAGA |
| | | ACTTTAAATGCATGG |
| | | GTAAAAGTAGTAGAA |
| | | GAGAAGGCTTTCAGC |
| | | CCAGAAGTGATACCC |
| | | ATGTTTTCAGCATTA |
| | | TCAGAAGGAGCCACC |
| | | CCACAAGATTTAAAC |
| | | ACCATGCTAAACACA |
| | | GTGGGGGGACATCAA |
| | | GCAGCCATGCAAATG |
| | | TTAAAAGAGACCATC |
| | | AATGAGGAAGCTGCA |
| | | GAATGGGATAGAGTG |
| | | CATCCAGTGCATGCA |
| | | GGGCCTATTGCACCA |
| | | GGCCAGATGAGAGAA |
| | | CCAAGGGGAAGTGAC |
| | | ATAGCAGGAACTACT |
| | | AGTACCCTTCAGGAA |
| | | CAAATAGGATGGATG |
| | | ACACATAATCCACCT |
| | | ATCCCAGTAGGAGAA |
| | | ATCTATAAAAGATGG |
| | | ATAATCCTGGGATTA |
| | | AATAAAATAGTAAGA |
| | | ATGTATAGCCCTACC |
| | | AGCATTCTGGACATA |
| | | AGACAAGGACCAAAG |
| | | GAACCCTTTAGAGAC |
| | | TATGTAGACCGATTC |
| | | TATAAAACTCTAAGA |
| | | GCCGAGCAAGCTTCA |
| | | CAAGAGGTAAAAAAT |
| | | TGGATGACAGAAACC |
| | | TTGTTGGTCCAAAAT |
| | | GCGAACCCAGATTGT |
| | | AAGACTATTTTAAAA |
| | | GCATTGGGACCAGGA |
| | | GCGACACTAGAAGAA |
| | | ATGATGACAGCATGT |
| | | CAGGGAGTGGGGGGA |
| | | CCCGGCCATAAAGCA |
| | | AGAGTTTTGGCTGAA |
| | | GCAATGAGCCAAGTA |
| | | ACAAATCCAGCTACC |
| | | ATAATGATACAGAAA |
| | | GGCAATTTTAGGAAC |
| | | CAAAGAAAGACTGTT |
| | | AAGTGTTTCAATTGT |
| | | GGCAAAGAAGGGCAC |
| | | ATAGCCAAAAATTGC |
| | | AGGGCCCCTAGGAAA |
| | | AAGGGCTGTTGGA |
| | | AATGTGGAAAGGAAG |
| | | GACACCAAATGAAAG |
| | | ATTGTACTGAGAGAC |
| | | AGGCTAATTTTTTAG |
| | | GGAAGATCTGGCCTT |
| | | CCCACAAGGGAAGGC |
| | | CAGGGAATTTTCTTC |
| | | AGAGCAGACCAGAGC |
| | | CAACAGCCCCACCAG |
| | | AAGAGAGCTTCAGGT |
| | | TTGGGGAAGAGACAA |
| | | CAACTCCCTCTCAGA |
| | | AGCAGGAGCCGATA |
| | | GACAAGGAACTGTAT |
| | | CCTTTAGCTTCCCTC |
| | | AGATCACTCTTTGGC |
| | | AGCGACCCCTCGTCA |
| | | CAATAA |
| 23 | HIV Pol | ATGAATTTGCCAGGA |
| | | AGATGGAAACCAAAA |
| | | ATGATAGGGGGAATT |
| | | GGAGGTTTTATCAAA |
| | | GTAGGACAGTATGAT |
| | | CAGATACTCATAGAA |
| | | ATCTGCGGACATAAA |
| | | GCTATAGGTACAGTA |
| | | TTAGTAGGACCTACA |
| | | CCTGTCAACATAATT |
| | | GGAAGAAATCTGTTG |
| | | ACTCAGATTGGCTGC |
| | | ACTTTAAATTTTCCC |
| | | ATTAGTCCTATTGAG |
| | | ACTGTACCAGTAAAA |
| | | TTAAAGCCAGGAATG |
| | | GATGGCCCAAAAGTT |
| | | AAACAATGGCCATTG |
| | | ACAGAAGAAAAAATA |
| | | AAAGCATTAGTAGAA |
| | | ATTTGTACAGAAATG |
| | | GAAAAGGAAGGAAAA |
| | | ATTTCAAAAATTGGG |
| | | CCTGAAAATCCATAC |
| | | AATACTCCAGTATTT |
| | | GCCATAAAGAAAAAA |
| | | GACAGTACTAAATGG |
| | | AGAAAATTAGTAGAT |
| | | TTCAGAGAACTTAAT |
| | | AAGAGAACTCAAGAT |
| | | TTCTGGGAAGTTCAA |
| | | TTAGGAATACCACAT |
| | | CCTGCAGGGTTAAAA |
| | | CAGAAAAAATCAGTA |
| | | ACAGTACTGGATGTG |
| | | GGCGATGCATATTTT |
| | | TCAGTTCCCTTAGAT |
| | | AAAGACTTCAGGAAG |
| | | TATACTGCATTTACC |
| | | ATACCTAGTATAAAC |
| | | AATGAGACACCAGGG |
| | | ATTAGATATCAGTAC |
| | | AATGTGCTTCCACAG |
| | | GGATGGAAAGGATCA |
| | | CCAGCAATATTCCAG |
| | | TGTAGCATGACAAAA |
| | | ATCTTAGAGCCTTTT |
| | | AGAAAACAAAATCCA |
| | | GACATAGTCATCTAT |
| | | CAATACATGGATGAT |
| | | TTGTATGTAGGATCT |
| | | GACTTAGAAATAGGG |
| | | CAGCATAGAACAAAA |
| | | ATAGAGGAACTGAGA |
| | | CAACATCTGTTGAGG |
| | | TGGGGATTTACCACA |
| | | CCAGACAAAAAACAT |
| | | CAGAAAGAACCTCCA |
| | | TTCCTTTGGATGGGT |
| | | TATGAACTCCATCCT |
| | | GATAAATGGACAGTA |
| | | CAGCCTATAGTGCTG |
| | | CCAGAAAAGGACAGC |
| | | TGGACTGTCAATGAC |
| | | ATACAGAAATTAGTG |
| | | GGAAAATTGAATTGG |
| | | GCAAGTCAGATTTAT |
| | | GCAGGGATTAAAGTA |
| | | AGGCAATTATGTAAA |
| | | CTTCTTAGGGGAACC |
| | | AAAGCACTAACAGAA |
| | | GTAGTACCACTAACA |
| | | GAAGAAGCAGAGCTA |
| | | GAACTGGCAGAAAAC |
| | | AGGGAGATTCTAAAA |
| | | GAACCGGTACATGGA |
| | | GTGTATTATGACCCA |
| | | TCAAAAGACTTAATA |
| | | GCAGAAATACAGAAG |
| | | CAGGGGCAAGGCCAA |
| | | TGGACATATCAAATT |
| | | TATCAAGAGCCATTT |
| | | AAAAATCTGAAAACA |
| | | GGAAAATATGCAAGA |
| | | ATGAAGGGTGCCCAC |
| | | ACTAATGATGTGAAA |
| | | CAATTAACAGAGGCA |
| | | GTACAAAAAATAGCC |
| | | ACAGAAAGCATAGTA |
| | | ATATGGGGAAAGACT |
| | | CCTAAATTTAAATTA |
| | | CCCATACAAAAGGAA |
| | | ACATGGGAAGCATGG |
| | | TGGACAGAGTATTGG |
| | | CAAGCCACCTGGATT |
| | | CCTGAGTGGGAGTTT |
| | | GTCAATACCCCTCCC |
| | | TTAGTGAAGTTATGG |
| | | TACCAGTTAGAGAAA |
| | | GAACCCATAATAGGA |
| | | GCAGAAACTTTCTAT |
| | | GTAGATGGGGCAGCC |
| | | AATAGGGAAACTAAA |
| | | TTAGGAAAAGCAGGA |
| | | TATGTAACTGACAGA |
| | | GGAAGACAAAAAGTT |
| | | GTCCCCCTAACGGAC |
| | | ACAACAAATCAGAAG |
| | | ACTGAGTTACAAGCA |
| | | ATTCATCTAGCTTTG |
| | | CAGGATTCGGGATTA |
| | | GAAGTAAACATAGTG |
| | | ACAGACTCACAATAT |
| | | GCATTGGGAATCATT |
| | | CAAGCACAACCAGAT |
| | | AAGAGTGAATCAGAG |
| | | TTAGTCAGTCAAATA |
| | | ATAGAGCAGTTAATA |
| | | AAAAAGGAAAAAGTC |
| | | TACCTGGCATGGGTA |
| | | CCAGCACACAAAGGA |
| | | ATTGGAGGAAATGAA |
| | | CAAGTAGATGGGTTG |
| | | GTCAGTGCTGGAATC |
| | | AGGAAAGTACTA |
| 24 | HIV Integrase | TTTTTAGATGGAATA |
| | (HIV Int) | GATAAGGCCCAAGAA |
| | | GAACATGAGAAATAT |
| | | CACAGTAATTGGAGA |
| | | GCAATGGCTAGTGAT |
| | | TTTAACCTACCACCT |
| | | GTAGTAGCAAAAGAA |
| | | ATAGTAGCCAGCTGT |
| | | GATAAATGTCAGCTA |
| | | AAAGGGGAAGCCATG |
| | | CATGGACAAGTAGAC |
| | | TGTAGCCCAGGAATA |
| | | TGGCAGCTAGATTGT |
| | | ACACATTTAGAAGGA |
| | | AAAGTTATCTTGGTA |
| | | GCAGTTCATGTAGCC |
| | | AGTGGATATATAGAA |
| | | GCAGAAGTAATTCCA |
| | | GCAGAGACAGGGCAA |
| | | GAAACAGCATACTTC |
| | | CTCTTAAAATTAGCA |
| | | GGAAGATGGCCAGTA |
| | | AAAACAGTACATACA |
| | | GACAATGGCAGCAAT |
| | | TTCACCAGTA |
| | | CTACAGTTAAGGCCG |
| | | CCTGTTGGTGGGCGG |
| | | GGATCAAGCAGGAAT |
| | | TTGGCATTCCCTACA |
| | | ATCCCCAAAGTCAAG |
| | | GAGTAATAGAATCTA |
| | | TGAATAAAGAATTAA |
| | | AGAAAATTATAGGAC |
| | | AGGTAAGAGATCAGG |
| | | CTGAACATCTTAAGA |
| | | CAGCAGTACAAATGG |
| | | CAGTATTCATCCACA |
| | | ATTTTAAAAGAAAAG |
| | | GGGGGATTGGGGGGT |
| | | ACAGTGCAGGGGAAA |
| | | GAATAGTAGACATAA |
| | | TAGCAACAGACATAC |
| | | AAACTAAAGAATTAC |
| | | AAAAACAAATTACAA |
| | | AAATTCAAAATTTTC |
| | | GGGTTTATTACAGGG |
| | | ACAGCAGAGATCCAG |
| | | TTTGGAAAGGACCAG |
| | | CAAAGCTCCTCTGGA |
| | | AAGGTGAAGGGGCAG |
| | | TAGTAATACAAGATA |
| | | ATAGTGACATAAAAG |
| | | TAGTGCCAAGAAGAA |
| | | AAGCAAAGATCATCA |
| | | GGGATTATGGAAAAC |
| | | AGATGGCAGGTGATG |
| | | ATTGTGTGGCAAGTA |
| | | GACAGGATGAGGATT |
| | | AA |
| 25 | HIV RRE | AGGAGCTTTGTTCCT |
| | | TGGGTTCTTGGGAGC |
| | | AGCAGGAAGCACTAT |
| | | GGGCGCAGCGTCAAT |
| | | GACGCTGACGGTACA |
| | | GGCCAGACAATTATT |
| | | GTCTGGTATAGTGCA |
| | | GCAGCAGAACAATTT |
| | | GCTGAGGGCTATTGA |
| | | GGCGCAACAGCATCT |
| | | GTTGCAACTCACAGT |
| | | CTGGGGCATCAAGCA |
| | | GCTCCAGGCAAGAAT |
| | | CCTGGCTGTGGAAAG |
| | | ATACCTAAAGGATCA |
| | | ACAGCTCCT |
| 26 | HIV Rev | ATGGCAGGAAGAAGC |
| | | GGAGACAGCGACGAA |
| | | GAACTCCTCAAGGCA |
| | | GTCAGACTCATCAAG |
| | | TTTCTCTATCAAAGC |
| | | AACCCACCTCCCAAT |
| | | CCCGAGGGGACCCGA |
| | | CAGGCCCGAAGGAAT |
| | | AGAAGAAGAAGGTGG |
| | | AGAGAGAGACAGAGA |
| | | CAGATCCATTCGATT |
| | | AGTGAACGGATCCTT |
| | | AGCACTTATCTGGGA |
| | | CGATCTGCGGAGCCT |
| | | GTGCCTCTTCAGCTA |
| | | CCACCGCTTGAGAGA |
| | | CTTACTCTTGATTGT |
| | | AACGAGGATTGTGGA |
| | | ACTTCTGGGACGCAG |
| | | GGGGTGGGAAGCCCT |
| | | CAAATATTGGTGGAA |
| | | TCTCCTACAATATTG |
| | | GAGTCAGGAGCTAAA |
| | | GAATAG |
| 27 | CMV | ACATTGATTATTGAC |
| | Promoter | TAGTTATTAATAGTA |
| | | ATCAATTACGGGGTC |
| | | ATTAGTTCATAGCCC |
| | | ATATATGGAGTTCCG |
| | | CGTTACATAACTTAC |
| | | GGTAAATGGCCCGCC |
| | | TGGCTGACCGCCCAA |
| | | CGACCCCCGCCCATT |
| | | GACGTCAATAATGAC |
| | | GTATGTTCCCATAGT |
| | | AACGCCAATAGGGAC |
| | | TTTCCATTGACGTCA |
| | | ATGGGTGGAGTATTT |
| | | ACGGTAAACTGCCCA |
| | | CTTGGCAGTACATCA |
| | | AGTGTATCATATGCC |
| | | AAGTACGCCCCCTAT |
| | | TGACGTCAATGACGG |
| | | TAAATGGCCCGCCTG |
| | | GCATTATGCCCAGTA |
| | | CATGACCTTATGGGA |
| | | CTTTCCTACTTGGCA |
| | | GTACATCTACGTATT |
| | | AGTCATCGCTATTAC |
| | | CATGGTGATGCGGTT |
| | | TTGGCAGTACATCAA |
| | | TGGGCGTGGATAGCG |
| | | GTTTGACTCACGGGG |
| | | ATTTCCAAGTCTCCA |
| | | CCCCATTGACGTCAA |
| | | TGGGAGTTTGTTTTG |
| | | GCACCAAAATCAACG |
| | | GGACTTTCCAAAATG |
| | | TCGTAACAACTCCGC |
| | | CCCATTGACGCAAAT |
| | | GGGCGGTAGGCGTG |
| | | TACGGTGGGAGGTCT |
| | | ATATAAGCAGAGCTC |
| | | TCTGGCTAACTAGAG |
| | | AACCCACTGCTTACT |
| | | G |
| 28 | Vesicular | ATGAAGTGCCTTTTG |
| | stomatitis | TACTTAGCCTTTTTA |
| | Indiana virus | TTCATTGGGGTGAAT |
| | glycoprotein | TGCAAGTTCACCATA |
| | VSV-G | GTTTTTCCACACAAC |
| | | CAAAAAGGAAACTGG |
| | | AAAAATGTTCCTTCT |
| | | AATTACCATTATTGC |
| | | CCGTCAAGCTCAGAT |
| | | TTAAATTGGCATAAT |
| | | GACTTAATAGGCACA |
| | | GCCTTACAAGTCAAA |
| | | ATGCCCAAGAGTCAC |
| | | AAGGCTATTCAAGCA |
| | | GACGGTTGGATGTGT |
| | | CATGCTTCCAAATGG |
| | | GTCACTACTTGTGAT |
| | | TTCCGCTGGTATGGA |
| | | CCGAAGTATATAACA |
| | | CATTCCATCCGATCC |
| | | TTCACTCCATCTGTA |
| | | GAACAATGCAAGGAA |
| | | AGCATTGAACAAACG |
| | | AAACAAGGAACTTGG |
| | | CTGAATCCAGGCTTC |
| | | CCTCCTCAAAGTTGT |
| | | GGATATGCAACTGTG |
| | | ACGGATGCCGAAGCA |
| | | GTGATTGTCCAGGTG |
| | | ACTCCTCACCATGTG |
| | | CTGGTTGATGAATAC |
| | | ACAGGAGAATGGGTT |
| | | GATTCACAGTTCATC |
| | | AACGGAAAATGCAGC |
| | | AATTACATATGCCCC |
| | | ACTGTCCATAACTCT |
| | | ACAACCTGGCATTCT |
| | | GACTATAAGGTCAAA |
| | | GGGCTATGTGATTCT |
| | | AACCTCATTTCCATG |
| | | GACATCACCTTCTTC |
| | | TCAGAGGACGGAGAG |
| | | CTATCATCCCTGGGA |
| | | AAGGAGGGCACAGGG |
| | | TTCAGAAGTAACTAC |
| | | TTTGCTTATGAAACT |
| | | GGAGGCAAGGC |
| | | CTGCAAAATGCAATA |
| | | CTGCAAGCATTGGGG |
| | | AGTCAGACTCCCATC |
| | | AGGTGTCTGGTTCGA |
| | | GATGGCTGATAAGGA |
| | | TCTCTTTGCTGCAGC |
| | | CAGATTCCCTGAATG |
| | | CCCAGAAGGGTCAAG |
| | | TATCTCTGCTCCATC |
| | | TCAGACCTCAGTGGA |
| | | TGTAAGTCTAATTCA |
| | | GGACGTTGAGAGGAT |
| | | CTTGGATTATTCCCT |
| | | CTGCCAAGAAACCTG |
| | | GAGCAAAATCAGAGC |
| | | GGGTCTTCCAATCTC |
| | | TCCAGTGGATCTCAG |
| | | CTATCTTGCTCCTAA |
| | | AAACCCAGGAACCGG |
| | | TCCTGCTTTCACCAT |
| | | AATCAATGGTACCCT |
| | | AAAATACTTTGAGAC |
| | | CAGATACATCAGAGT |
| | | CGATATTGCTGCTCC |
| | | AATCCTCTCAAGAAT |
| | | GGTCGGAATGATCAG |
| | | TGGAACTACCACAGA |
| | | AAGGGAACTGTGGGA |
| | | TGACTGGGCACCATA |
| | | TGAAGACGTGGAAAT |
| | | TGGACCCAATGGAGT |
| | | TCTGAGGACCAGTTC |
| | | AGGATATAAGTTTCC |
| | | TTTATACATGATTGG |
| | | ACATGGTATGTTGGA |
| | | CTCCGATCTTCATCT |
| | | TAGCTCAAAGGCTCA |
| | | GGTGTTCGAACATCC |
| | | TCACATTCAAGACGC |
| | | TGCTTCGCAACTTCC |
| | | TGATGATGAGAGTTT |
| | | ATTTTTTGGTGATAC |
| | | TGGGCTATCCAAAAA |
| | | TCCAATCGAGCTTGT |
| | | AGAAGGTTGGTTCAG |
| | | TAGTTGGAAAAGCTC |
| | | TATTGCCTCTTTTTT |
| | | CTTTATCATAGGGTT |
| | | AATCATTGGACTATT |
| | | CTTGGTTCTCCGAGT |
| | | TGGTATCCATCTTTG |
| | | CATTAAATTAAAGCA |
| | | CACCAAGAAAAGACA |
| | | GATTTATACAGACAT |
| | | AGAGATGAACCGACT |
| | | TGGAAAGTGA |
| 29 | Left ITR | CCTGCAGGCAGCTGC |
| | | GCGCTCGCTCGCTCA |
| | | CTGAGGCCGCCCGGG |
| | | CAAAGCCCGGGCGTC |
| | | GGGCGACCTTTGGTC |
| | | GCCCGGCCTCAGTGA |
| | | GCGAGCGAGCGCGCA |
| | | GAGAGGGAGTGGCCA |
| | | ACTCCATCACTAGGG |
| | | GTTCCT |
| 30 | Poly A | GACTGTGCCTTCTAG |
| | Element | TTGCCAGCCATCTGT |
| | | TGTTTGCCCCTCCCC |
| | | CGTGCCTTCCTTGAC |
| | | CCTGGAAGGTGCCAC |
| | | TCCCACTGTCCTTTC |
| | | CTAATAAAATGAGGA |
| | | AATTGCATCGCATTG |
| | | TCTGAGTAGGTGTCA |
| | | TTCTATTCTGGGGGG |
| | | TGGGGTGGGGCAGGA |
| | | CAGCAAGGGGGAGGA |
| | | TTGGGAAGACAATAG |
| | | CAGGCATGCTGGGGA |
| | | TGCGGTGGGCTCTAT |
| | | GGC |
| 31 | Right ITR | AGGAACCCCTAGTGA |
| | | TGGAGTTGGCCACTC |
| | | CCTCTCTGCGCGCTC |
| | | GCTCGCTCACTGAGG |
| | | CCGGGCGACCAAAGG |
| | | TCGCCCGACGCCCGG |
| | | GCTTTGCCCGGGCGG |
| | | CCTCAGTGAGCGAGC |
| | | GAGCGCGCAGCTGCC |
| | | TGCAGG |
| 32 | E2A Element | TTAAAAGTCGAAGGG |
| | | GTTCTCGCGCTCGTC |
| | | GTTGTGCGCCGCGCT |
| | | GGGGAGGGCCACGTT |
| | | GCGGAACTGGTACTT |
| | | GGGCTGCCACTTGAA |
| | | CTCGGGGATCACCAG |
| | | TTTGGGCACTGGGGT |
| | | CTCGGGGAAGGTCTC |
| | | GCTCCACATGCGCCG |
| | | GCTCATCTGCAGGGC |
| | | GCCCAGCATGTCAGG |
| | | CGCGGAGATCTTGAA |
| | | ATCGCAGTTGGGGCC |
| | | GGTGCTCTGCGCGCG |
| | | CGAGTTGCGGTACAC |
| | | TGGGTTGCAGCACTG |
| | | GAACACCATCAGACT |
| | | GGGGTACTTCACACT |
| | | AGCCAGCACGCTCTT |
| | | GTCGCTGATCTGATC |
| | | CTTGTCCAGGTCCTC |
| | | GGCGTTGCTCAGGCC |
| | | GAACGGGGTCATCTT |
| | | GCACAGCTGGCGGCC |
| | | CAGGAAGGGCACGCT |
| | | CTGAGGCTTGTGGTT |
| | | ACACTCGCAGTGCAC |
| | | GGGCATCAGCATCAT |
| | | CCCCGCGCCGCGCTG |
| | | CATATTCGGGTAGAG |
| | | GGCCTTGACGAAGGC |
| | | CGCGATCTGCTTGAA |
| | | AGCTTGCTGGGCCTT |
| | | GGCCCCCTCGCTGAA |
| | | AAACAGGCCGCAGCT |
| | | CTTCCCGCTGAACTG |
| | | ATTATTCCCGCACCC |
| | | GGCATCATGGACGCA |
| | | GCAGCGCGCGTCATG |
| | | GCTGGTCAGTTGCAC |
| | | CACGCTCCGTCCCCA |
| | | GCGGTTCTGGGTCAC |
| | | CTTGGCCTTGCTGGG |
| | | TTGCTCCTTCAGCGC |
| | | ACGCTGCCCGTTCTC |
| | | ACTGGTCACATCCAT |
| | | CTCCACCACGTGGTC |
| | | CTTGTGGATCATCAC |
| | | CGTCCCATGCAGACA |
| | | CTTGAGCTGGCCTTC |
| | | CACCTCGGTGCAGCC |
| | | GTGGTCCCACAGGGC |
| | | ACTGCCGGTGCACTC |
| | | CCAGTTCTTGTGCGC |
| | | GATCCCGCTGTGGCT |
| | | GAAGATGTAACCTTG |
| | | CAACAGGCGACCCAT |
| | | GATGGTGCTAAAGCT |
| | | CTTCTGGGTGGTGAA |
| | | GGTCAGTTGCAGACC |
| | | GCGGGCCTCCTCGTT |
| | | CATCCAGGTCTGGCA |
| | | CATCTTTTGGAAGAT |
| | | CTCGGTCTGCTCGGG |
| | | CATGAGCTTGTAAGC |
| | | ATCGCGCAGGCCGCT |
| | | GTCGACGCGGTAGCG |
| | | TTCCATCAGCACATT |
| | | CATGGTATCCATGCC |
| | | CTTCTCCCAGGACGA |
| | | GACCAGAGG |
| | | CAGACTCAGGGGGTT |
| | | GCGCACGTTCAGGAC |
| | | ACCGGGGGTCGCGGG |
| | | CTCGACGATGCGTTT |
| | | TCCGTCCTTGCCTTC |
| | | CTTCAACAGAACCGG |
| | | CGGCTGGCTGAATCC |
| | | CACTCCCACGATCAC |
| | | GGCTTCTTCCTGGGG |
| | | CATCTCTTCGTCTGG |
| | | GTCTACCTTGGTCAC |
| | | ATGCTTGGTCTTTCT |
| | | GGCTTGCTCCGGATC |
| | | CCACCCGCTGATACT |
| | | TTCGGCGCTTGGTTG |
| | | GCAGAGGAGGTGGCG |
| | | GCGAGGGGCTCCTCT |
| | | CCTGCTCCGGCGGAT |
| | | AGCGCGCTGAACCGT |
| | | GGCCCCGGGGCGGAG |
| | | TGGCCTCTCGGTCCA |
| | | TGAACCGGCGCACGT |
| | | CCTGACTGCCGCCGG |
| | | CCAT |
| 33 | E4 element | TCATGTATCTTTATT |
| | | GATTTTTACACCAGC |
| | | ACGGGTAGTCAGTCT |
| | | CCCACCACCAGCCCA |
| | | TTTCACAGTGTAAAC |
| | | AATTCTCTCAGCACG |
| | | GGTGGCCTTAAATAG |
| | | GGCAATATTCTGATT |
| | | AGTGCGGGAACTGGA |
| | | CTTGGGGTCTATAAT |
| | | CCACACAGTTTCCTG |
| | | GCGAGCCAAACGGGG |
| | | GTCGGTGATTGAGAT |
| | | GAAGCCGTCCTCTGA |
| | | AAAGTCATCCAAGCG |
| | | AGCCTCACAGTCCAA |
| | | GGTCACAGTATTATG |
| | | ATAATCTGCATGATC |
| | | ACAATCGGGCAACAG |
| | | GGGATGTTGTTCAGT |
| | | CAGTGAAGCCCTGGT |
| | | TTCCTCATCAGATCG |
| | | TGGTAAACGGGCCCT |
| | | GCGATATGGATGATG |
| | | GCGGAGCGAGCTGGA |
| | | TTGAATCTCGGTTTG |
| | | CAT |
| 34 | VARNA | AGCGGGCACTCTTCC |
| | | GTGGTCTGGTGGATA |
| | | AATTCGCAAGGGTAT |
| | | CATGGCGGACGACCG |
| | | GGGTTCGAGCCCCGT |
| | | ATCCGGCCGTCCGCC |
| | | GTGATCCATGCGGTT |
| | | ACCGCCCGCGTGTCG |
| | | AACCCAGGTGTGCGA |
| | | CGTCAGACAACGGGG |
| | | GAGTGCTCCTTT |
| 35 | AAV2 Rep | ATGGCTGCCGATGGT |
| | | TATCTTCCAGATTGG |
| | | CTCGAGGACACTCTC |
| | | TCTGAAGGAATAAGA |
| | | CAGTGGTGGAAGCTC |
| | | AAACCTGGCCCACCA |
| | | CCACCAAAGCCCGCA |
| | | GAGCGGCATAAGGAC |
| | | GACAGCAGGGGTCTT |
| | | GTGCTTCCTGGGTAC |
| | | AAGTACCTCGGACCC |
| | | TTCAACGGACTCGAC |
| | | AAGGGAGAGCCGGTC |
| | | AACGAGGCAGACGCC |
| | | GCGGCCCTCGAGCAC |
| | | GACAAAGCCTACGAC |
| | | CGGCAGCTCGACAGC |
| | | GGAGACAACCCGTAC |
| | | CTCAAGTACAACCAC |
| | | GCCGACGCGGAGTTT |
| | | CAGGAGCGCCTTAAA |
| | | GAAGATACGTCTTTT |
| | | GGGGGCAACCTCGGA |
| | | CGAGCAGTCTTCCAG |
| | | GCGAAAAAGAGGGTT |
| | | CTTGAACCTCTGGGC |
| | | CTGGTTGAGGAACCT |
| | | GTTAAGACGGCTCCG |
| | | GGAAAAAAGAGGCCG |
| | | GTAGAGCACTCTCCT |
| | | GTGGAGCCAGACTCC |
| | | TCCTCGGGAACCGGA |
| | | AAGGCGGGCCAGCAG |
| | | CCTGCAAGAAAAAGA |
| | | TTGAATTTTGGTCAG |
| | | ACTGGAGACGCAGAC |
| | | TCAGTACCTGACCCC |
| | | CAGCCTCTCGGACAG |
| | | CCACCAGCAGCCCCC |
| | | TCTGGTCTGGGAACT |
| | | AATACGATGGCTACA |
| | | GGCAGTGGCGCACCA |
| | | ATGGCAGACAATAAC |
| | | GAGGGCGCCGACGGA |
| | | GTGGGTAATTCCTCG |
| | | GGAAATTGGCATTGC |
| | | GATTCCACATGGATG |
| | | GGCGACAGAGTCATC |
| | | ACCACCAGCACCCGA |
| | | ACCTGGGCCCTGCCC |
| | | ACCTACAACAACCAC |
| | | CTCTACAAACAAATT |
| | | TCCAGCCAATCAGGA |
| | | GCCTCGAACGACAAT |
| | | CACTACTTTGGCTAC |
| | | AGCACCCCTTGGGGG |
| | | TATTTTGACTTCAAC |
| | | AGATTCCACTGCCAC |
| | | TTTTCACCACGTGAC |
| | | TGGCAAAGACTCATC |
| | | AACAACAACTGGGGA |
| | | TTCCGACCCAAGAGA |
| | | CTCAACTTCAAGCTC |
| | | TTTAACATTCAAGTC |
| | | AAAGAGGTCACGCAG |
| | | AATGACGGTACGACG |
| | | ACGATTGCCAATAAC |
| | | CTTACCAGCACGGTT |
| | | CAGGTGTTTACTGAC |
| | | TCGGAGTACCAGCTC |
| | | CCGTACGTCCTCGGC |
| | | TCGGCGCATCAAGGA |
| | | TGCCTCCCGCCGTTC |
| | | CCAGCAGACGTCTTC |
| | | ATGGTGCCACAGTAT |
| | | GGATACCTCACCCTG |
| | | AACAACGGGAGTCAG |
| | | GCAGTAGGACGCTCT |
| | | TCATTTTACTGCCTG |
| | | GAGTACTTTCCTTCT |
| | | CAGATGCTGCGTACC |
| | | GGAAACAACTTTACC |
| | | TTCAGCTACACTTTT |
| | | GAGGACGTTCCTTTC |
| | | CACAGCAGCTACGCT |
| | | CACAGCCAGAGTCTG |
| | | GACCGTCTCATGAAT |
| | | CCTCTCATCGACCAG |
| | | TACCTGTATTACTTG |
| | | AGCAGAACAAACACT |
| | | CCAAGTGGAACCACC |
| | | ACGCAGTCAAGGCTT |
| | | CAGTTTTCTCAGGCC |
| | | GGAGCGAGTGACATT |
| | | CGGGACCAGTCTAGG |
| | | AACTGGCTTCCTGGA |
| | | CCCTGTTACCGCCAG |
| | | CAGCGAGTATCAAAG |
| | | ACATCTGCGGATAAC |
| | | AAC |
| | | AACAGTGAATACTCG |
| | | TGGACTGGAGCTACC |
| | | AAGTACCACCTCAAT |
| | | GGCAGAGACTCTCTG |
| | | GTGAATCCGGGCCCG |
| | | GCCATGGCAAGCCAC |
| | | AAGGAAGCAAGGCTC |
| | | AGAGAAAACAAATGT |
| | | GGACATTGAAAAGGT |
| | | CATGATTACAGACGA |
| | | AGAGGAAATCAGGAC |
| | | AACCAATCCCGTGGC |
| | | TACGGAGCAGTATGG |
| | | TTCTGTATCTACCAA |
| | | CCTCCAGAGAGGCAA |
| | | CAGACAAGCAGCTAC |
| | | CGCAGATGTCAACAC |
| | | ACAAGGCGTTCTTCC |
| | | AGGCATGGTCTGGCA |
| | | GGACAGAGATGTGTA |
| | | CCTTCAGGGGCCCAT |
| | | CTGGGCAAAGATTCC |
| | | ACACACGGACGGACA |
| | | TTTTCACCCCTCTCC |
| | | CCTCATGGGTGGATT |
| | | CGGACTTAAACACCC |
| | | TCCTCCACAGATTCT |
| | | CATCAAGAACACCCC |
| | | GGTACCTGCGAATCC |
| | | TTCGACCACCTTCAG |
| | | TGCGGCAAAGTTTGC |
| | | TTCCTTCATCACACA |
| | | GTACTCCACGGGACA |
| | | GGTCAGCGTGGAGAT |
| | | CGAGTGGGAGCTGCA |
| | | GAAGGAAAACAGCAA |
| | | ACGCTGGAATCCCGA |
| | | AATTCAGTACACTTC |
| | | CAACTACAACAAGTC |
| | | TGTTAATGTGGACTT |
| | | TACTGTGGACACTAA |
| | | TGGCGTGTATTCAGA |
| | | GCCTCGCCCCATTGG |
| | | CACCAGATACCTGAC |
| | | TCGTAATCTGTAA |
| 36 | AAV2 Cap | ATGCCGGGGTTTTAC |
| | | GAGATTGTGATTAAG |
| | | GTCCCCAGCGACCTT |
| | | GACGAGCATCTGCCC |
| | | GGCATTTCTGACAGC |
| | | TTTGTGAACTGGGTG |
| | | GCCGAGAAGGAATGG |
| | | GAGTTGCCGCCAGAT |
| | | TCTGACATGGATCTG |
| | | AATCTGATTGAGCAG |
| | | GCACCCCTGACCGTG |
| | | GCCGAGAAGCTGCAG |
| | | CGCGACTTTCTGACG |
| | | GAATGGCGCCGTGTG |
| | | AGTAAGGCCCCGGAG |
| | | GCCCTTTTCTTTGTG |
| | | CAATTTGAGAAGGGA |
| | | GAGAGCTACTTCCAC |
| | | ATGCACGTGCTCGTG |
| | | GAAACCACCGGGGTG |
| | | AAATCCATGGTTTTG |
| | | GGACGTTTCCTGAGT |
| | | CAGATTCGCGAAAAA |
| | | CTGATTCAGAGAATT |
| | | TACCGCGGGATCGAG |
| | | CCGACTTTGCCAAAC |
| | | TGGTTCGCGGTCACA |
| | | AAGACCAGAAATGGC |
| | | GCCGGAGGCGGGAAC |
| | | AAGGTGGTGGATGAG |
| | | TGCTACATCCCCAAT |
| | | TACTTGCTCCCCAAA |
| | | ACCCAGCCTGAGCTC |
| | | CAGTGGGCGTGGACT |
| | | AATATGGAACAGTAT |
| | | TTAAGCGCCTGTTTG |
| | | AATCTCACGGAGCGT |
| | | AAACGGTTGGTGGCG |
| | | CAGCATCTGACGCAC |
| | | GTGTCGCAGACGCAG |
| | | GAGCAGAACAAAGAG |
| | | AATCAGAATCCCAAT |
| | | TCTGATGCGCCGGTG |
| | | ATCAGATCAAAAACT |
| | | TCAGCCAGGTACATG |
| | | GAGCTGGTCGGGTGG |
| | | CTCGTGGACAAGGGG |
| | | ATTACCTCGGAGAAG |
| | | CAGTGGATCCAGGAG |
| | | GACCAGGCCTCATAC |
| | | ATCTCCTTCAATGCG |
| | | GCCTCCAACTCGCGG |
| | | TCCCAAATCAAGGCT |
| | | GCCTTGGACAATGCG |
| | | GGAAAGATTATGAGC |
| | | CTGACTAAAACCGCC |
| | | CCCGACTACCTGGTG |
| | | GGCCAGCAGCCCGTG |
| | | GAGGACATTTCCAGC |
| | | AATCGGATTTATAAA |
| | | ATTTTGGAACTAAAC |
| | | GGGTACGATCCCCAA |
| | | TATGCGGCTTCCGTC |
| | | TTTCTGGGATGGGCC |
| | | ACGAAAAAGTTCGGC |
| | | AAGAGGAACACCATC |
| | | TGGCTGTTTGGGCCT |
| | | GCAACTACCGGGAAG |
| | | ACCAACATCGCGGAG |
| | | GCCATAGCCCACACT |
| | | GTGCCCTTCTACGGG |
| | | TGCGTAAACTGGACC |
| | | AATGAGAACTTTCCC |
| | | TTCAACGACTGTGTC |
| | | GACAAGATGGTGATC |
| | | TGGTGGGAGGAGGGG |
| | | AAGATGACCGCCAAG |
| | | GTCGTGGAGTCGGCC |
| | | AAAGCCATTCTCGGA |
| | | GGAAGCAAGGTGCGC |
| | | GTGGACCAGAAATGC |
| | | AAGTCCTCGGCCCAG |
| | | ATAGACCCGACTCCC |
| | | GTGATCGTCACCTCC |
| | | AACACCAACATGTGC |
| | | GCCGTGATTGACGGG |
| | | AACTCAACGACCTTC |
| | | GAACACCAGCAGCCG |
| | | TTGCAAGACCGGATG |
| | | TTCAAATTTGAACTC |
| | | ACCCGCCGTCTGGAT |
| | | CATGACTTTGGGAAG |
| | | GTCACCAAGCAGGAA |
| | | GTCAAAGACTTTTTC |
| | | CGGTGGGCAAAGGAT |
| | | CACGTGGTTGAGGTG |
| | | GAGCATGAATTCTAC |
| | | GTCAAAAAGGGTGGA |
| | | GCCAAGAAAAGACCC |
| | | GCCCCCAGTGACGCA |
| | | GATATAAGTGAGCCC |
| | | AAACGGGTGCGCGAG |
| | | TCAGTTGCGCAGCCA |
| | | TCGACGTCAGACGCG |
| | | GAAGCTTCGATCAAC |
| | | TACGCAGACAGGTAC |
| | | CAAAACAAATGTTCT |
| | | CGTCACGTGGGCATG |
| | | AATCTGATGCTGTTT |
| | | CCCTGCAGACAATGC |
| | | GAGAGAATGAATCAG |
| | | AATTCAAATATCTGC |
| | | TTCACTCACGGACAG |
| | | AAAGACTGTTTAGAG |
| | | TGCTTTCCCGTGTCA |
| | | GAATCTCAACCCGTT |
| | | TCTGTCGTCAAAAAG |
| | | GCGTATCAGAAACTG |
| | | TGCTACATTCATCAT |
| | | ATCATGGGAAAGGTG |
| | | CCAGACGCTTG |
| | | CACTGCCTGCGATCT |
| | | GGTCAATGTGGATTT |
| | | GGATGACTGCATCTT |
| | | TGAACAATAA |
| 37 | AAV8 Cap | ATGGCTGCAGGCGGT |
| | | GGCGCACCAATGGCA |
| | | GACAATAACGAAGGC |
| | | GCCGACGGAGTGGGT |
| | | AGTTCCTCGGGAAAT |
| | | TGGCATTGCGATTCC |
| | | ACATGGCTGGGCGAC |
| | | AGAGTCATCACCACC |
| | | AGCACCCGAACCTGG |
| | | GCCCTGCCCACCTAC |
| | | AACAACCACCTCTAC |
| | | AAGCAAATCTCCAAC |
| | | GGGACATCGGGAGGA |
| | | GCCACCAACGACAAC |
| | | ACCTACTTCGGCTAC |
| | | AGCACCCCCTGGGGG |
| | | TATTTTGACTTTAAC |
| | | AGATTCCACTGCCAC |
| | | TTTTCACCACGTGAC |
| | | TGGCAGCGACTCATC |
| | | AACAACAACTGGGGA |
| | | TTCCGGCCCAAGAGA |
| | | CTCAGCTTCAAGCTC |
| | | TTCAACATCCAGGTC |
| | | AAGGAGGTCACGCAG |
| | | AATGAAGGCACCAAG |
| | | ACCATCGCCAATAAC |
| | | CTCACCAGCACCATC |
| | | CAGGTGTTTACGGAC |
| | | TCGGAGTACCAGCTG |
| | | CCGTACGTTCTCGGC |
| | | TCTGCCCACCAGGGC |
| | | TGCCTGCCTCCGTTC |
| | | CCGGCGGACGTGTTC |
| | | ATGATTCCCCAGTAC |
| | | GGCTACCTAACACTC |
| | | AACAACGGTAGTCAG |
| | | GCCGTGGGACGCTCC |
| | | TCCTTCTACTGCCTG |
| | | GAATACTTTCCTTCG |
| | | CAGATGCTGAGAACC |
| | | GGCAACAACTTCCAG |
| | | TTTACTTACACCTTC |
| | | GAGGACGTGCCTTTC |
| | | CACAGCAGCTACGCC |
| | | CACAGCCAGAGCTTG |
| | | GACCGGCTGATGAAT |
| | | CCTCTGATTGACCAG |
| | | TACCTGTACTACTTG |
| | | TCTCGGACTCAAACA |
| | | ACAGGAGGCACGGCA |
| | | AATACGCAGACTCTG |
| | | GGCTTCAGCCAAGGT |
| | | GGGCCTAATACAATG |
| | | GCCAATCAGGCAAAG |
| | | AACTGGCTGCCAGGA |
| | | CCCTGTTACCGCCAA |
| | | CAACGCGTCTCAACG |
| | | ACAACCGGGCAAAAC |
| | | AACAATAGCAACTTT |
| | | GCCTGGACTGCTGGG |
| | | ACCAAATACCATCTG |
| | | AATGGAAGAAATTCA |
| | | TTGGCTAATCCTGGC |
| | | ATCGCTATGGCAACA |
| | | CACAAAGACGACGAG |
| | | GAGCGTTTTTTTCCC |
| | | AGTAACGGGATCCTG |
| | | ATTTTTGGCAAACAA |
| | | AATGCTGCCAGAGAC |
| | | AATGCGGATTACAGC |
| | | GATGTCATGCTCACC |
| | | AGCGAGGAAGAAATC |
| | | AAAACCACTAACCCT |
| | | GTGGCTACAGAGGAA |
| | | TACGGTATCGTGGCA |
| | | GATAACTTGCAGCAG |
| | | CAAAACACGGCTCCT |
| | | CAAATTGGAACTGTC |
| | | AACAGCCAGGGGGCC |
| | | TTACCCGGTATGGTC |
| | | TGGCAGAACCGGGAC |
| | | GTGTACCTGCAGGGT |
| | | CCCATCTGGGCCAAG |
| | | ATTCCTCACACGGAC |
| | | GGCAACTTCCACCCG |
| | | TCTCCGCTGATGGGC |
| | | GGCTTTGGCCTGAAA |
| | | CATCCTCCGCCTCAG |
| | | ATCCTGATCAAGAAC |
| | | ACGCCTGTACCTGCG |
| | | GATCCTCCGACCACC |
| | | TTCAACCAGTCAAAG |
| | | CTGAACTCTTTCATC |
| | | ACGCAATACAGCACC |
| | | GGACAGGTCAGCGTG |
| | | GAAATTGAATGGGAG |
| | | CTGCAGAAGGAAAAC |
| | | AGCAAGCGCTGGAAC |
| | | CCCGAGATCCAGTAC |
| | | ACCTCCAACTACTAC |
| | | AAATCTACAAGTGTG |
| | | GACTTTGCTGTTAAT |
| | | ACAGAAGGCGTGTAC |
| | | TCTGAACCCCGCCCC |
| | | ATTGGCACCCGTTAC |
| | | CTCACCCGTAATCTG |
| | | TAA |
| 38 | AAV DJ Cap | ATGGCTGCCGATGGT |
| | | TATCTTCCAGATTGG |
| | | CTCGAGGACACTCTC |
| | | TCTGAAGGAATAAGA |
| | | CAGTGGTGGAAGCTC |
| | | AAACCTGGCCCACCA |
| | | CCACCAAAGCCCGCA |
| | | GAGCGGCATAAGGAC |
| | | GACAGCAGGGGTCTT |
| | | GTGCTTCCTGGGTAC |
| | | AAGTACCTCGGACCC |
| | | TTCAACGGACTCGAC |
| | | AAGGGAGAGCCGGTC |
| | | AACGAGGCAGACGCC |
| | | GCGGCCCTCGAGCAC |
| | | GACAAAGCCTACGAC |
| | | CGGCAGCTCGACAGC |
| | | GGAGACAACCCGTAC |
| | | CTCAAGTACAACCAC |
| | | GCCGACGCCGAGTT |
| | | CCAGGAGCGGCTCAA |
| | | AGAAGATACGTCTTT |
| | | TGGGGGCAACCTCGG |
| | | GCGAGCAGTCTTCCA |
| | | GGCCAAAAAGAGGCT |
| | | TCTTGAACCTCTTGG |
| | | TCTGGTTGAGGAAGC |
| | | GGCTAAGACGGCTCC |
| | | TGGAAAGAAGAGGCC |
| | | TGTAGAGCACTCTCC |
| | | TGTGGAGCCAGACTC |
| | | CTCCTCGGGAACCGG |
| | | AAAGGCGGGCCAGCA |
| | | GCCTGCAAGAAAAAG |
| | | ATTGAATTTTGGTCA |
| | | GACTGGAGACGCAGA |
| | | CTCAGTCCCAGACCC |
| | | TCAACCAATCGGAGA |
| | | ACCTCCCGCAGCCCC |
| | | CTCAGGTGTGGGATC |
| | | TCTTACAATGGCTGC |
| | | AGGCGGTGGCGCACC |
| | | AATGGCAGACAATAA |
| | | CGAGGGCGCCGACGG |
| | | AGTGGGTAATTCCTC |
| | | GGGAAATTGGCATTG |
| | | CGATTCCACATGGAT |
| | | GGGCGACAGAGTCAT |
| | | CACCACCAGCACCCG |
| | | AACCTG |
| | | GGCCCTGCCCACCTA |
| | | CAACAACCACCTCTA |
| | | CAAGCAAATCTCCAA |
| | | CAGCACATCTGGAGG |
| | | ATCTTCAAATGACAA |
| | | CGCCTACTTCGGCTA |
| | | CAGCACCCCCTGGGG |
| | | GTATTTTGACTTTAA |
| | | CAGATTCCACTGCCA |
| | | CTTTTCACCACGTGA |
| | | CTGGCAGCGACTCAT |
| | | CAACAACAACTGGGG |
| | | ATTCCGGCCCAAGAG |
| | | ACTCAGCTTCAAGCT |
| | | CTTCAACATCCAGGT |
| | | CAAGGAGGTCACGCA |
| | | GAATGAAGGCACCAA |
| | | GACCATCGCCAATAA |
| | | CCTCACCAGCACCAT |
| | | CCAGGTGTTTACGGA |
| | | CTCGGAGTACCAGCT |
| | | GCCGTACGTTCTCGG |
| | | CTCTGCCCACCAGGG |
| | | CTGCCTGCCTCCGTT |
| | | CCCGGCGGACGTGTT |
| | | CATGATTCCCCAGTA |
| | | CGGCTACCTAACACT |
| | | CAACAACGGTAGTCA |
| | | GGCCGTGGGACGCTC |
| | | CTCCTTCTACTGCCT |
| | | GGAATACTTTCCTTC |
| | | GCAGATGCTGAGAAC |
| | | CGGCAACAACTTCCA |
| | | GTTTACTTACACCTT |
| | | CGAGGACGTGCCTTT |
| | | CCACAGCAGCTACGC |
| | | CCACAGCCAGAGCTT |
| | | GGACCGGCTGATGAA |
| | | TCCTCTGATTGACCA |
| | | GTACCTGTACTACTT |
| | | GTCTCGGACTCAAAC |
| | | AACAGGAGGCACGAC |
| | | AAATACGCAGACTCT |
| | | GGGCTTCAGCCAAGG |
| | | TGGGCCTAATACAAT |
| | | GGCCAATCAGGCAAA |
| | | GAACTGGCTGCCAGG |
| | | ACCCTGTTACCGCCA |
| | | GCAGCGAGTATCAAA |
| | | GACATCTGCGGATAA |
| | | CAACAACAGTGAATA |
| | | CTCGTGGACTGGAGC |
| | | TACCAAGTACCACCT |
| | | CAATGGCAGAGACTC |
| | | TCTGGTGAATCCGGG |
| | | CCCGGCCATGGCAAG |
| | | CCACAAGGACGATGA |
| | | AGAAAAGTTTTTTCC |
| | | TCAGAGCGGGGTTCT |
| | | CATCTTTGGGAAGCA |
| | | AGGCTCAGAGAAAAC |
| | | AAATGTGGACATTGA |
| | | AAAGGTCATGATTAC |
| | | AGACGAAGAGGAAAT |
| | | CAGGACAACCAATCC |
| | | CGTGGCTACGGAGCA |
| | | GTATGGTTCTGTATC |
| | | TACCAACCTCCAGAG |
| | | AGGCAACAGACAAGC |
| | | AGCTACCGCAGATGT |
| | | CAACACACAAGGCGT |
| | | TCTTCCAGGCATGGT |
| | | CTGGCAGGACAGAGA |
| | | TGTGTACCTTCAGGG |
| | | GCCCATCTGGGCAAA |
| | | GATTCCACACACGGA |
| | | CGGACATTTTCACCC |
| | | CTCTCCCCTCATGGG |
| | | TGGATTCGGACTTAA |
| | | ACACCCTCCGCCTCA |
| | | GATCCTGATCAAGAA |
| | | CACGCCTGTACCTGC |
| | | GGATCCTCCGACCAC |
| | | CTTCAACCAGTCAAA |
| | | GCTGAACTCTTTCAT |
| | | CACCCAGTATTCTAC |
| | | TGGCCAAGTCAGCGT |
| | | GGAGATCGAGTGGGA |
| | | GCTGCAGAAGGAAAA |
| | | CAGCAAGCGCTGGAA |
| | | CCCCGAGATCCAGTA |
| | | CACCTCCAACTACTA |
| | | CAAATCTACAAGTGT |
| | | GGACTTTGCTGTTAA |
| | | TACAGAAGGCGTGTA |
| | | CTCTGAACCCCGCCC |
| | | CATTGGCACCCGTTA |
| | | CCTCACCCGTAATCT |
| | | GTAA |
| 39 | Chicken bela | GGAGTCGCTGCGTTG |
| | actin intron | CCTTCGCCCCGTGCC |
| | | CCGCTCCGCGCCGCC |
| | | TCGCGCCGCCCGCCC |
| | | CGGCTCTGACTGACC |
| | | GCGTTACTCCCACAG |
| | | GTGAGCGGGCGGGAC |
| | | GGCCCTTCTCCTCCG |
| | | GGCTGTAATTAGCGC |
| | | TTGGTTTAATGACGG |
| | | CTCGTTTCTTTTCTG |
| | | TGGCTGCGTGAAAGC |
| | | CTTAAAGGGCTCCGG |
| | | GAGGGCCCTTTGTGC |
| | | GGGGGGGAGCGGCTC |
| | | GGGGGGTGCGTGCGT |
| | | GTGTGTGTGCGTGGG |
| | | GAGCGCCGCGTGCGG |
| | | CCCGCGCTGCCCGGC |
| | | GGCTGTGAGCGCTGC |
| | | GGGCGCGGCGCGGGG |
| | | CTTTGTGCGCTCCGC |
| | | GTGTGCGCGAGGGGA |
| | | GCGCGGCCGGGGGCG |
| | | GTGCCCCGCGGTGCG |
| | | GGGGGGCTGCGAGGG |
| | | GAACAAAGGCTGCGT |
| | | GCGGGGTGTGTGCGT |
| | | GGGGGGGTGAGCAGG |
| | | GGGTGTGGGCGCGGC |
| | | GGTCGGGCTGTAACC |
| | | CCCCCCTGCACCCCC |
| | | CTCCCCGAGTTGCTG |
| | | AGCACGGCCCGGCTT |
| | | CGGGTGCGGGGCTCC |
| | | GTGCGGGGCGTGGCG |
| | | CGGGGCTCGCCGTGC |
| | | CGGGCGGGGGGTGGC |
| | | GGCAGGTGGGGGTGC |
| | | CGGGCGGGGCGGGGC |
| | | CGCCTCGGGCCGGGG |
| | | AGGGCTCGGGGGAGG |
| | | GGCGCGGCGGCCCCG |
| | | GAGCGCCGGCGGCTG |
| | | TCGAGGCGCGGCGAG |
| | | CCGCAGCCATTGCCT |
| | | TTTATGGTAATCGTG |
| | | CGAGAGGGCGCAGGG |
| | | ACTTCCTTTGTCCCA |
| | | AATCTGGCGGAGCCG |
| | | AAATCTGGGAGGCGC |
| | | CGCCGCACCCCCTCT |
| | | AGCGGGCGCGGGCGA |
| | | AGCGGTGCGGCGCCG |
| | | GCAGGAAGGAAATGG |
| | | GCGGGGAGGGCCTT |
| | | CGTGCGTCGCCGCGC |
| | | CGCCGTCCCCTTCTC |
| | | CATCTCCAGCCTCGG |
| | | GGCTGCCGCAGGGGG |
| | | ACGGCTGCCTTCGGG |
| | | GGGGACGGGGCAGGG |
| | | CGGGGTTCGGCTTCT |
| | | GGCGTGTGACCGGCG |
| | | G |
| 40 | Rabbit beta | AGATCTTTTTCCCTC |
| | globin pols A | TGCCAAAAATTATGG |
| | | GGACATCATGAAGCC |
| | | CCTTGAGCATCTGAC |
| | | TTCTGGCTAATAAAG |
| | | GAAATTTATTTTCAT |
| | | TGCAATAGTGTGTTG |
| | | GAATTTTTTGTGTCT |
| | | CTCACTCGGAAGGAC |
| | | ATATGGGAGGGCAAA |
| | | TCATTTAAAACATCA |
| | | GAATGAGTATTTGGT |
| | | TTAGAGTTTGGCAAC |
| | | ATATGCCATATGCTG |
| | | GCTGCCATGAACAAA |
| | | GGTGGCTATAAAGAG |
| | | GTCATCAGTATATGA |
| | | AACAGCCCCCTGCTG |
| | | TCCATTCCTTATTCC |
| | | ATAGAAAAGCCTTGA |
| | | CTTGAGGTTAGATTT |
| | | TTTTTATATTTTGTT |
| | | TTGTGTTATTTTTTT |
| | | CTTTAACATCCCTAA |
| | | AATTTTCCTTACATG |
| | | TTTTACTAGCCAGAT |
| | | TTTTCCTCCTCTCCT |
| | | GACTACTCCCAGTCA |
| | | TAGCTGTCCCTCTTC |
| | | TCTTATGAAGATC |
| 41 | Forward | TAAGCAGAATTCATG |
| | Primer | AATTTGCCAGGAAGA |
| | | T |
| 42 | Reverse | CCATACAATGAATGG |
| | Primer | ACACTAGGCGGCCGC |
| | | ACGAAT |
| 43 | Gag, Pol, | GAATTCATGAATTTG |
| | Intcgrasc | CCAGGAAGATGGAAA |
| | fragment | CCAAAAATGATAGGG |
| | | GGAATTGGAGGTTTT |
| | | ATCAAAGTAAGACAG |
| | | TATGATCAGATACTC |
| | | ATAGAAATCTGCGGA |
| | | CATAAAGCTATAGGT |
| | | ACAGTATTAGTAGGA |
| | | CCTACACCTGTCAAC |
| | | ATAATTGGAAGAAAT |
| | | CTGTTGACTCAGATT |
| | | GGCTGCACTTTAAAT |
| | | TTTCCCATTAGTCCT |
| | | ATTGAGACTGTACCA |
| | | GTAAAATTAAAGCCA |
| | | GGAATGGATGGCCCA |
| | | AAAGTTAAACAATGG |
| | | CCATTGACAGAAGAA |
| | | AAAATAAAAGCATTA |
| | | GTAGAAATTTGTACA |
| | | GAAATGGAAAAGGAA |
| | | GGAAAAATTTCAAAA |
| | | ATTGGGCCTGAAAAT |
| | | CCATACAATACTCCA |
| | | GTATTTGCCATAAAG |
| | | AAAAAAGACAGTACT |
| | | AAATGGAGAAAATTA |
| | | GTAGATTTCAGAGAA |
| | | CTTAATAAGAGAACT |
| | | CAAGATTTCTGGGAA |
| | | GTTCAATTAGGAATA |
| | | CCACATCCTGCAGGG |
| | | TTAAAACAGAAAAAA |
| | | TCAGTAACAGTACTG |
| | | GATGTGGGCGATGCA |
| | | TATTTTTCAGTTCCC |
| | | TTAGATAAAGACTTC |
| | | AGGAAGTATACTGCA |
| | | TTTACCATACCTAGT |
| | | ATAAACAATGAGACA |
| | | CCAGGGATTAGATAT |
| | | CAGTACAATGTGCTT |
| | | CCACAGGGATGGAAA |
| | | GGATCACCAGCAATA |
| | | TTCCAGTGTAGCATG |
| | | ACAAAAATCTTAGAG |
| | | CCTTTTAGAAAACAA |
| | | AATCCAGACATAGTC |
| | | ATCTATCAATACATG |
| | | GATGATTTGTATGTA |
| | | GGATCTGACTTAGAA |
| | | ATAGGGCAGCATAGA |
| | | ACAAAAATAGAGGAA |
| | | CTGAGACAACATCTG |
| | | TTGAGGTGGGGATTT |
| | | ACCACACCAGACAAA |
| | | AAACATCAGAAAGAA |
| | | CCTCCATTCCTTTGG |
| | | ATGGGTTATGAACTC |
| | | CATCCTGATAAATGG |
| | | ACAGTACAGCCTATA |
| | | GTGCTGCCAGAAAAG |
| | | GACAGCTGGACTGTC |
| | | AATGACATACAGAAA |
| | | TTAGTGGGAAAATTG |
| | | AATTGGGCAAGTCAG |
| | | ATTTATGCAGGGATT |
| | | AAAGTAAGGCAATTA |
| | | TGTAAACTTCTTAGG |
| | | GGAACCAAAGCACTA |
| | | ACAGAAGTAGTACCA |
| | | CTAACAGAAGAAGCA |
| | | GAGCTAGAACTGGCA |
| | | GAAAACAGGGAGATT |
| | | CTAAAAGAACCGGTA |
| | | CATGGAGTGTATTAT |
| | | GACCCATCAAAAGAC |
| | | TTAATAGCAGAAATA |
| | | CAGAAGCAGGGGCAA |
| | | GGCCAATGGACATAT |
| | | CAAATTTATCAAGAG |
| | | CCATTTAAAAATCTG |
| | | AAAACAGGAAAGTAT |
| | | GCAAGAATGAAGGGT |
| | | GCCCACACTAATGAT |
| | | GTGAAACAATTAACA |
| | | GAGGCAGTACAAAAA |
| | | ATAGCCACAGAAAGC |
| | | ATAGTAATATGGGGA |
| | | AAGACTCCTAAATTT |
| | | AAATTACCCATACAA |
| | | AAGGAAACATGGGAA |
| | | GCATGGTGGACAGAG |
| | | TATTGGCAAGCCACC |
| | | TGGATTCCTGAGTGG |
| | | GAGTTTGTCAATACC |
| | | CCTCCCTTAGTGAAG |
| | | TTATGGTACCAGTTA |
| | | GAGAAAGAACCCATA |
| | | ATAGGAGCAGAAACT |
| | | TTCTATGTAGATGGG |
| | | GCAGCCAATAGGGAA |
| | | ACTAAATTAGGAAAA |
| | | GCAGGATATGTAACT |
| | | GACAGAGGAAGACAA |
| | | AAAGTTGTCCCCCTA |
| | | ACGGACACAACAAAT |
| | | CAGAAGACTGAGTTA |
| | | CAAGCAATTCATCTA |
| | | GCTTTGCAGGATTCG |
| | | GGATTAGAAGTAAAC |
| | | ATAGTGACAGACTCA |
| | | CAATATGCATTGGGA |
| | | ATCATTCAAGCACAA |
| | | CCAGATAAGAGTGAA |
| | | TCAGAGTTAGTCAGT |
| | | CAAATAATAGAGCAG |
| | | TTAATAAAAAAGGAA |
| | | AAAGTCTACCTGGCA |
| | | TGGGTACCAGCACAC |
| | | AAAGGAATTGGAGGA |
| | | AATGAACAAGTAGAT |
| | | AAATTGGTCAGTGCT |
| | | GGAATCAGGAAAGTA |
| | | CTATTTTTAGATGGA |
| | | ATAGATAAGGCCCAA |
| | | GAAGAACATGAGAAA |
| | | TATCACAGTAATTGG |
| | | AGAGCA |
| | | ATGGCTAGTGATTTT |
| | | AACCTACCACCTGTA |
| | | GTAGCAAAAGAAATA |
| | | GTAGCCAGCTGTGAT |
| | | AAATGTCAGCTAAAA |
| | | GGGGAAGCCATGCAT |
| | | GGACAAGTAGACTGT |
| | | AGCCCAGGAATATGG |
| | | CAGCTAGATTGTACA |
| | | CATTTAGAAGGAAAA |
| | | GTTATCTTGGTAGCA |
| | | GTTCATGTAGCCAGT |
| | | GGATATATAGAAGCA |
| | | GAAGTAATTCCAGCA |
| | | GAGACAGGGCAAGAA |
| | | ACAGCATACTTCCTC |
| | | TTAAAATTAGCAGGA |
| | | AGATGGCCAGTAAAA |
| | | ACAGTACATACAGAC |
| | | AATGGCAGCAATTTC |
| | | ACCAGTACTACAGTT |
| | | AAGGCCGCCTGTTGG |
| | | TGGGCGGGGATCAAG |
| | | CAGGAATTTGGCATT |
| | | CCCTACAATCCCCAA |
| | | AGTCAAGGAGTAATA |
| | | GAATCTATGAATAAA |
| | | GAATTAAAGAAAATT |
| | | ATAGGACAGGTAAGA |
| | | GATCAGGCTGAACAT |
| | | CTTAAGACAGCAGTA |
| | | CAAATGGCAGTATTC |
| | | ATCCACAATTTTAAA |
| | | AGAAAAGGGGGGATT |
| | | GGGGGGTACAGTGCA |
| | | GGGGAAAGAATAGTA |
| | | GACATAATAGCAACA |
| | | GACATACAAACTAAA |
| | | GAATTACAAAAACAA |
| | | ATTACAAAAATTCAA |
| | | AATTTTCGGGTTTAT |
| | | TACAGGGACAGCAGA |
| | | GATCCAGTTTGGAAA |
| | | GGACCAGCAAAGCTC |
| | | CTCTGGAAAGGTGAA |
| | | GGGGCAGTAGTAATA |
| | | CAAGATAATAGTGAC |
| | | ATAAAAGTAGTGCCA |
| | | AGAAGAAAAGCAAAG |
| | | ATCATCAGGGATTAT |
| | | GGAAAACAGATGGCA |
| | | GGTGATGATTGTGTG |
| | | GCAAGTAGACAGGAT |
| | | GAGGATTAA |
| 44 | DNA | TCTAGAATGGCAGGA |
| | Fragment | AGAAGCGGAGACAGC |
| | containing the | GACGAAGAGCTCATC |
| | RRE, REV, | AGAACAGTCAGACTC |
| | and rabbit beta | ATCAAGCTTCTCTAT |
| | globin | CAAAGCAACCCACCT |
| | poly A | CCCAATCCCGAGGGG |
| | sequence | ACCCGACAGGCCCGA |
| | | AGGAATAGAAGAAGA |
| | | AGGTGGAGAGAGAGA |
| | | CAGAGACAGATCCAT |
| | | TCGATTAGTGAACGG |
| | | ATCCTTGGCACTTAT |
| | | CTGGGACGATCTGCG |
| | | GAGCCTGTGCCTCTT |
| | | CAGCTACCACCGCTT |
| | | GAGAGACTTACTCTT |
| | | GATTGTAACGAGGAT |
| | | TGTGGAACTTCTGGG |
| | | ACGCAGGGGGTGGGA |
| | | AGCCCTCAAATATTG |
| | | GTGGAATCTCCTACA |
| | | ATATTGGAGTCAGGA |
| | | GCTAAAGAATAGAGG |
| | | AGCTTTGTTCCTTGG |
| | | GTTCTTGGGAGCAGC |
| | | AGGAAGCACTATGGG |
| | | CGCAGCGTCAATGAC |
| | | GCTGACGGTACAGGC |
| | | CAGACAATTATTGTC |
| | | TGGTATAGTGCAGCA |
| | | GCAGAACAATTTGCT |
| | | GAGGGCTATTGAGGC |
| | | GCAACAGCATCTGTT |
| | | GCAACTCACAGTCTG |
| | | GGGCATCAAGCAGCT |
| | | CCAGGCAAGAATCCT |
| | | GGCTGTGGAAAGATA |
| | | CCTAAAGGATCAACA |
| | | GCTCCTAGATCTTTT |
| | | TCCCTCTGCCAAAAA |
| | | TTATGGGGACATCAT |
| | | GAAGCCCCTTGAGCA |
| | | TCTGACTTCTGGCTA |
| | | ATAAAGGAAATTTAT |
| | | TTTCATTGCAATAGT |
| | | GTGTTGGAATTTTTT |
| | | GTGTCTCTCACTCGG |
| | | AAGGACATATGGGAG |
| | | GGCAAATCATTTAAA |
| | | ACATCAGAATGAGTA |
| | | TTTGGTTTAGAGTTT |
| | | GGCAACATATGCCAT |
| | | ATGCTGGCTGCCATG |
| | | AACAAAGGTGGCTAT |
| | | AAAGAGGTCATCAGT |
| | | ATATGAAACAGCCCC |
| | | CTGCTGTCCATTCCT |
| | | TATTCCATAGAAAAG |
| | | CCTTGACTTGAGGTT |
| | | AGATTTTTTTTATAT |
| | | TTTGTTTTGTGTTAT |
| | | TTTTTTCTTTAACAT |
| | | CCCTAAAATTTTCCT |
| | | TACATGTTTTACTAG |
| | | CCAGATTTTTCCTCC |
| | | TCTCCTGACTACTCC |
| | | CAGTCATAGCTGTCC |
| | | CTCTTCTCTTATGAA |
| | | GATCCCTCGACCTGC |
| | | AGCCCAAGCTTGGCG |
| | | TAATCATGGTCATAG |
| | | CTGTTTCCTGTGTGA |
| | | AATTGTTATCCGCTC |
| | | ACAATTCCACACAAC |
| | | ATACGAGCCGGAAGC |
| | | ATAAAGTGTAAAGCC |
| | | TGGGGTGCCTAATGA |
| | | GTGAGCTAACTCACA |
| | | TTAATTGCGTTGCGC |
| | | TCACTGCCCGCTTTC |
| | | CAGTCGGGAAACCTG |
| | | TCGTGCCAGCGGATC |
| | | CGCATCTCAATTAGT |
| | | CAGCAACCATAGTCC |
| | | CGCCCCTAACTCCGC |
| | | CCATCCCGCCCCTAA |
| | | CTCCGCCCAGTTCCG |
| | | CCCATTCTCCGCCCC |
| | | ATGGCTGACTAATTT |
| | | TTTTTATTTATGCAG |
| | | AGGCCGAGGCCGCCT |
| | | CGGCCTCTGAGCTAT |
| | | TCCAGAAGTAGTGAG |
| | | GAGGCTTTTTTGGAG |
| | | GCCTAGGCTTTTGCA |
| | | AAAAGCTAACTTGTT |
| | | TATTGCAGCTTATAA |
| | | TGGTTACAAATAAAG |
| | | CAATAGCATCACATC |
| | | CAAACTCATCAATGT |
| | | ATCTTATCAGCGGCC |
| | | GCCCCGGG |
| 45 | DNA | ACGCGTTAGTTATTA |
| | fragment | ATAGTAATCAATTAC |
| | Containing | GGGGTCATTAGTTCA |
| | the | TAGCCCATATATGGA |
| | CAG | GTTCCGCGTTACATA |
| | enhancer/ | ACTTACGGTAAATGG |
| | promoter | CCCGCCTGGCTGACC |
| | intron | GCCCAACGACCCCCG |
| | sequence | CCCATTGACGTCAAT |
| | | AATGACGTATGTTCC |
| | | CATAGTAACGCCAAT |
| | | AGGGACTTTCCATTG |
| | | ACGTCAATGGGTGGA |
| | | CTATTTACGGTAAAC |
| | | TGCCCACTTGGCAGT |
| | | ACATCAAGTGTATCA |
| | | TATGCCAAGTACGCC |
| | | CCCTATTGACGTCAA |
| | | TGACGGTAAATGGCC |
| | | CGCCTGGCATTATGC |
| | | CCAGTACATGACCTT |
| | | ATGGGACTTTCCTAC |
| | | TTGGCAGTACATCTA |
| | | CGTATTAGTCATCGC |
| | | TATTACCATGGGTCG |
| | | AGGTGAGCCCCACGT |
| | | TCTGCTTCACTCTCC |
| | | CCATCTCCCCCCCCT |
| | | CCCCACCCCCAATTT |
| | | TGTATTTATTTATTT |
| | | TTTAATTATTTTGTG |
| | | CAGCGATGGGGGCGG |
| | | GGGGGGGGGGGGCGC |
| | | GCGCCAGGCGGGGCG |
| | | GGGCGGGGCGAGGGG |
| | | CGGGGCGGGGCGAGG |
| | | CGGAGAGGTGCGGCG |
| | | GCAGCCAATCAGAGC |
| | | GGCGCGCTCCGAAAG |
| | | TTTCCTTTTATGGCG |
| | | AGGCGGCGGCGGCGG |
| | | CGGCCCTATAAAAAG |
| | | CGAAGCGCGCGGCGG |
| | | GCGGGAGTCGCTGCG |
| | | TTGCCTTCGCCCCGT |
| | | GCCCCGCTCCGCGCC |
| | | GCCTCGCGCCGCCCG |
| | | CCCCGGCTCTGACTG |
| | | ACCGCGTTACTCCCA |
| | | CAGGTGAGCGGGCGG |
| | | GACGGCCCTTCTCCT |
| | | CCGGGCTGTAATTAG |
| | | CGCTTGGTTTAATGA |
| | | CGGCTCGTTTCTTTT |
| | | CTGTGGCTGCGTGAA |
| | | AGCCTTAAAGGGCTC |
| | | CGGGAGGGCCCTTTG |
| | | TGCGGGGGGGAGCGG |
| | | CTCGGGGGGTGCGTG |
| | | CGTGTGTGTGTGCGT |
| | | GGGGAGCGCCGCGTG |
| | | CGGCCCGCGCTGCCC |
| | | GGCGGCTGTGAGCGC |
| | | TGCGGGCGCGGCGCG |
| | | GGGCTTTGTGCGCTC |
| | | CGCGTGTGCGCGAGG |
| | | GGAGCGCGGCCGGGG |
| | | GCGGTGCCCCGCGGT |
| | | GCGGGGGGGCTGCGA |
| | | GGGGAACAAAGGCTG |
| | | CGTGCGGGGTGTGTG |
| | | CGTGGGGGGGTGAGC |
| | | AGGGGGTGTGGGCGC |
| | | GGCGGTCGGGCTGTA |
| | | ACCCCCCCCTGCACC |
| | | CCCCTCCCCGAGTTG |
| | | CTGAGCACGGCCCGG |
| | | CTTCGGGTGCGGGGC |
| | | TCCGTGCGGGGCGTG |
| | | GCGCGGGGCTCGCCG |
| | | TGCCGGGCGGGGGGT |
| | | GGCGGCAGGTGGGGG |
| | | TGCCGGGCGGGGCGG |
| | | GGCCGCCTCGGGCCG |
| | | GGGAGGGCTCGGGGG |
| | | AGGGGCGCGGCGGCC |
| | | CCGGAGCGCCGGCGG |
| | | CTGTCGAGGCGCGGC |
| | | GAGCCGCAGCCATTG |
| | | CCTTTTATGGTAATC |
| | | GTGCGAGAGGGCGCA |
| | | GGGACTTCCTTTGTC |
| | | CCAAATCTGGCGGAG |
| | | CCGAAATCTGGGAGG |
| | | CGCCGCCGCACCCCC |
| | | TCTAGCGGGCGCGGG |
| | | CGAAGCGGTGCGGCG |
| | | CCGGCAGGAAGGAAA |
| | | TGGGCGGGGAGGGCC |
| | | TTCGTGCGTCGCCGC |
| | | GCCGCCGTCCCCTTC |
| | | TCCATCTCCAGCCTC |
| | | GGGGCTGCCGCAGGG |
| | | GGACGGCTGCCTTCG |
| | | GGGGGGACGGGGCAG |
| | | GGCGGGGTTCGGCTT |
| | | CTGGCGTGTGACCGG |
| | | CGGGAATTC |
| 46 | RSV promoter | CAATTGCGATGTACG |
| | and HIV Rev | GGCCAGATATACGCG |
| | | TATCTGAGGGGACTA |
| | | GGGTGTGTTTAGGCG |
| | | AAAAGCGGGGCTTCG |
| | | GTTGTACGCGGTTAG |
| | | GAGTCCCCTCAGGAT |
| | | ATAGTAGTTTCGCTT |
| | | TTGCATAGGGAGGGG |
| | | GAAATGTAGTCTTAT |
| | | GCAATACACTTGTAG |
| | | TCTTGCAACATGGTA |
| | | ACGATGAGTTAGCAA |
| | | CATGCCTTACAAGGA |
| | | GAGAAAAAGCACCGT |
| | | GCATGCCGATTGGTG |
| | | GAAGTAAGGTGGTAC |
| | | GATCGTGCCTTATTA |
| | | GGAAGGCAACAGACA |
| | | GGTCTGACATGGATT |
| | | GGACGAACCACTGAA |
| | | TTCCGCATTGCAGAG |
| | | ATAATTGTATTTAAG |
| | | TGCCTAGCTCGATAC |
| | | AATAAACGCCATTTG |
| | | ACCATTCACCACATT |
| | | GGTGTGCACCTCCAA |
| | | GCTCGAGCTCGTTTA |
| | | GTGAACCGTCAGATC |
| | | GCCTGGAGACGCCAT |
| | | CCACGCTGTTTTGAC |
| | | CTCCATAGAAGACAC |
| | | CGGGACCGATCCAGC |
| | | CTCCCCTCGAAGCTA |
| | | GCGATTAGGCATCTC |
| | | CTATGGCAGGAAGAA |
| | | GCGGAGACAGCGACG |
| | | AAGAACTCCTCAAGG |
| | | CAGTCAGACTCATCA |
| | | AGTTTCTCTATCAAA |
| | | GCAACCCACCTCCCA |
| | | ATCCCGAGGGGACCC |
| | | GACAGGCCCGAAGGA |
| | | ATAGAAGAAGAAGGT |
| | | GGAGAGAGAGACAGA |
| | | GACAGATCCATTCGA |
| | | TTAGTGAACGGATCC |
| | | TTAGCACTTATCTGG |
| | | GACGATCTGCGGAGC |
| | | CTGTGCCTCTTCAGC |
| | | TACCACCGCTTGAGA |
| | | GACTTACTCTTGATT |
| | | GTAACGAGGATTGTG |
| | | GAACTTCTGGGACGC |
| | | AGGGGGTGGGAAGCC |
| | | CTCAAATATTGGTGG |
| | | AATCTCCTACAATAT |
| | | TGGAGTCAGGAGCTA |
| | | AAGAATAGTCTAGA |
| 47 | Elongation | CCGGTGCCTAGAGAA |
| | Factor-1 alpha | GGTGGCGCGGGGTAA |
| | (EF-1 alpha) | ACTGGGAAAGTGATG |
| | promoter | TCGTGTACTGGCTCC |
| | | GCCTTTTTCCCGAGG |
| | | GTGGGGGAGAACCGT |
| | | ATATAAGTGCAGTAG |
| | | TCGCCGTGAACGTTC |
| | | TTTTTCGCAACGGGT |
| | | TTGCCGCCAGAACAC |
| | | AGGTAAGTGCCGTGT |
| | | GTGGTTCCCGCGGGC |
| | | CTGGCCTCTTTACGG |
| | | GTTATGGCCCTTGCG |
| | | TGCCTTGAATTACTT |
| | | CCACGCCCCTGGCTG |
| | | CAGTACGTGATTCTT |
| | | GATCCCGAGCTTCGG |
| | | GTTGGAAGTGGGTGG |
| | | GAGAGTTCGAGGCCT |
| | | TGCGCTTAAGGAGCC |
| | | CCTTCGCCTCGTGCT |
| | | TGAGTTGAGGCCTGG |
| | | CCTGGGCGCTGGGGC |
| | | CGCCGCGTGCGAATC |
| | | TGGTGGCACCTTCGC |
| | | GCCTGTCTCGCTGCT |
| | | TTCGATAAGTCTCTA |
| | | GCTAGTCTTGTAAAT |
| | | GCGGGGCAAGATCTG |
| | | CACACTGGTATTTCG |
| | | GTTTTTGGGGCCGCG |
| | | GGCGGCGACGGGGCC |
| | | CGTGCGTCCCAGCGC |
| | | ACATGTTCGGCGAGG |
| | | CGGGGCCTGCGAGCG |
| | | CGGCCACCGAGAATC |
| | | GGACGGGGGTAGTCT |
| | | CAAGCTGGCCGGCCT |
| | | GCTCTGGTGCCTGGC |
| | | CTCGCGCCGCCGTGT |
| | | ATCGCCCCGCCCTGG |
| | | GCGGCAAGGCTGGCC |
| | | CGGTCGGCACCAGTT |
| | | GCGTGAGCGGAAAGA |
| | | TGGCCGCTTCCCGGC |
| | | CCTGCTGCAGGGAGC |
| | | TCAAAATGGAGGACG |
| | | CGGCGCTCGGGAGAG |
| | | CGGGCGGGTGAGTCA |
| | | CCCACACAAAGGAAA |
| | | AGGGCCTTTCCGTCC |
| | | TCAGCCGTCGCTTCA |
| | | TGTGACTCCACGGAG |
| | | TACCGGGCGCCGTCC |
| | | AGGCACCTCGATTAG |
| | | TTCTCGAGCTTTTGG |
| | | AGTACGTCGTCTTTA |
| | | GGTTGGGGGGAGGGG |
| | | TTTTATGCGATGGAG |
| | | TTTCCCCACACTGAG |
| | | TGGGTGGAGACTGAA |
| | | GTTAGGCCAGCTTGG |
| | | CACTTGATGTAATTC |
| | | TCCTTGGAATTTGCC |
| | | CTTTTTGAGTTTGGA |
| | | TCTTGGTTCATTCTC |
| | | AAGCCTCAGACAGTG |
| | | GTTCAAAGTTTTTTT |
| | | CTTCCATTTCAGGTG |
| | | TCGTGA |
| 48 | PGK Promoter | GGGGTTGGGGTTGCG |
| | | CCTTTTCCAAGGCAG |
| | | CCCTGGGTTTGCGCA |
| | | GGGACGCGGCTGCTC |
| | | TGGGCGTGGTTCCGG |
| | | GAAACGCAGCGGCGC |
| | | CGACCCTGGGTCTCG |
| | | CACATTCTTCACGTC |
| | | CGTTCGCAGCGTCAC |
| | | CCGGATCTTCGCCGC |
| | | TACCCTTGTGGGCCC |
| | | CCCGGCGACGCTTCC |
| | | TGCTCCGCCCCTAAG |
| | | TCGGGAAGGTTCCTT |
| | | GCGGTTCGCGGCGTG |
| | | CCGGACGTGACAAAC |
| | | GGAAGCCGCACGTCT |
| | | CACTAGTACCCTCGC |
| | | AGACGGACAGCGCCA |
| | | GGGAGCAATGGCAGC |
| | | GCGCCGACCGCGATG |
| | | GGCTGTGGCCAATAG |
| | | CGGCTGCTCAGCAGG |
| | | GCGCGCCGAGAGCAG |
| | | CGGCCGGGAAGGGGC |
| | | GGTGCGGGAGGCGGG |
| | | GTGTGGGGCGGTAGT |
| | | GTGGGCCCTGTTCCT |
| | | GCCCGCGCGGTGTTC |
| | | CGCATTCTGCAAGCC |
| | | TCCGGAGCGCACGTC |
| | | GGCAGTCGGCTCCCT |
| | | CGTTGACCGAATCAC |
| | | CGACCTCTCTCCCCA |
| | | G |
| 49 | UbC Promoter | GCGCCGGGTTTTGGC |
| | | GCCTCCCGCGGGCGC |
| | | CCCCCTCCTCACGGC |
| | | GAGCGCTGCCACGTC |
| | | AGACGAAGGGCGCAG |
| | | GAGCGTTCCTGATCC |
| | | TTCCGCCCGGACGCT |
| | | CAGGACAGCGGCCCG |
| | | CTGCTCATAAGACTC |
| | | GGCCTTAGAACCCCA |
| | | GTATCAGCAGAAGGA |
| | | CATTTTAGGACGGGA |
| | | CTTGGGTGACTCTAG |
| | | GGCACTGGTTTTCTT |
| | | TCCAGAGAGCGGAAC |
| | | AGGCGAGGAAAAGTA |
| | | GTCCCTTCTCGGCGA |
| | | TTCTGCGGAGGGATC |
| | | TCCGTGGGGCGGTGA |
| | | ACGCCGATGATTATA |
| | | TAAGGACGCGCCGGG |
| | | TGTGGCACAGCTAGT |
| | | TCCGTCGCAGCCGGG |
| | | ATTTGGGTCGCGGTT |
| | | CTTGTTTGTGGATCG |
| | | CTGTGATCGTCACTT |
| | | GGTGAGTTGCGGGCT |
| | | GCTGGGCTGGCCGGG |
| | | GCTTTCGTGGCCGCC |
| | | GGGCCGCTCGGTGGG |
| | | ACGGAAGCGTGTGGA |
| | | GAGACCGCCAAGGGC |
| | | TGTAGTCTGGGTCCG |
| | | CGAGCAAGGTTGCCC |
| | | TGAACTGGGGGTTGG |
| | | GGGGAGCGCACAAAA |
| | | TGGCGGCTGTTCCCG |
| | | AGTCTTGAATGGAAG |
| | | ACGCTTGTAAGGCGG |
| | | GCTGTGAGGTCGTTG |
| | | AAACAAGGTGGGGGG |
| | | CATGGTGGGCGGCAA |
| | | GAACCCAAGGTCTTG |
| | | AGGCCTTCGCTAATG |
| | | CGGGAAAGCTCTTAT |
| | | TCGGGTGAGATGGGC |
| | | TGGGGCACCATCTGG |
| | | GGACCCTGACGTGAA |
| | | GTTTGTCACTGACTG |
| | | GAGAACTCGGGTTTG |
| | | TCGTCTGGTTGCGGG |
| | | GGCGGCAGTTATGCG |
| | | GTGCCGTTGGGCAGT |
| | | GCACCCGTACCTTTG |
| | | GGAGCGCGCGCCTCG |
| | | TCGTGTCGTGACGTC |
| | | ACCCGTTCTGTTGGC |
| | | TTATAATGCAGGGTG |
| | | GGGCCACCTGCCGGT |
| | | AGGTGTGCGGTAGGC |
| | | TTTTCTCCGTCGCAG |
| | | GACGCAGGGTTCGGG |
| | | CCTAGGGTAGGCTCT |
| | | CCTGAATCGACAGGC |
| | | GCCGGACCTCTGGTG |
| | | AGGGGAGGGATAAGT |
| | | GAGGCGTCAGTTTCT |
| | | TTGGTCGGTTTTATG |
| | | TACCTATCTTCTTAA |
| | | GTAGCTGAAGCTCCG |
| | | GTTTTGAACTATGCG |
| | | CTCGGGGTTGGCGAG |
| | | TGTGTTTTGTGAAGT |
| | | TTTTTAGGCACCTTT |
| | | TGAAATGTAATCATT |
| | | TGGGTCAATATGTAA |
| | | TTTTCAGTGTTAGAC |
| | | TAGTAAA |
| 50 | SV40 Poly A | GTTTATTGCAGCTTA |
| | | TAATGGTTACAAATA |
| | | AAGCAATAGCATCAC |
| | | AACCAAACTCATCAA |
| | | TGTATCTTATCA |
| 51 | bHG Poly A | GACTGTGCCTTCTAG |
| | | TTGCCAGCCATCTGT |
| | | TGTTTGCCCCTCCCC |
| | | CGTGCCTTCCTTGAC |
| | | CCTGGAAGGTGCCAC |
| | | TCCCACTGTCCTTTC |
| | | CTAATAAAATGAGGA |
| | | AATTGCATCGCATTG |
| | | TCTGAGTAGGTGTCA |
| | | TTCTATTCTGGGGGG |
| | | TGGGGTGGGGCAGGA |
| | | CAGCAAGGGGGAGGA |
| | | TTGGGAAGACAATAG |
| | | CAGGCATGCTGGGGA |
| | | TGCGGTGGGCTCTAT |
| | | GG |
| 52 | RD114 | ATGAAACTCCCAACA |
| | Envelope | GGAATGGTCATTTTA |
| | | TGTAGCCTAATAATA |
| | | GTTCGGGCAGGGTTT |
| | | GACGACCCCCGCAAG |
| | | GCTATCGCATTAGTA |
| | | CAAAAACAACATGGT |
| | | AAACCATGCGAATGC |
| | | AGCGGAGGGCAGGTA |
| | | TCCGAGGCCCCACCG |
| | | AACTCCATCCAACAG |
| | | GTAACTTGCCCAGGC |
| | | AAGACGGCCTACTTA |
| | | ATGACCAACCAAAAA |
| | | TGGAAATGCAGAGTC |
| | | ACTCCAAAAAATCTC |
| | | ACCCCTAGCGGGGGA |
| | | GAACTCCAGAACTGC |
| | | CCCTGTAACACTTTC |
| | | CAGGACTCGATGCAC |
| | | AGTTCTTGTTATACT |
| | | GAATACCGGCAATGC |
| | | AGGGCGAATAATAAG |
| | | ACATACTACACGGCC |
| | | ACCTTGCTTAAAATA |
| | | CGGTCTGGGAGCCTC |
| | | AACGAGGTACAGATA |
| | | TTACAAAACCCCAAT |
| | | CAGCTCCTACAGTCC |
| | | CCTTGTAGGGGCTCT |
| | | ATAAATCAGCCCGTT |
| | | TGCTGGAGTGCCACA |
| | | GCCCCCATCCATATC |
| | | TCCGATGGTGGAGGA |
| | | CCCCTCGATACTAAG |
| | | AGAGTGTGGACAGTC |
| | | CAAAAAAGGCTAGAA |
| | | CAAATTCATAAGGCT |
| | | ATGCATCCTGAACTT |
| | | CAATACCACCCCTTA |
| | | GCCCTGCCCAAAGTC |
| | | AGAGATGACCTTAGC |
| | | CTTGATGCACGGACT |
| | | TTTGATATCCTGAAT |
| | | ACCACTTTTAGGTTA |
| | | CTCCAGATGTCCAAT |
| | | TTTAGCCTTGCCCAA |
| | | GATTGTTGGCTCTGT |
| | | TTAAAACTAGGTACC |
| | | CCTACCCCTCTTGCG |
| | | ATACCCACTCCCTCT |
| | | TTAACCTACTCCCTA |
| | | GCAGACTCCCTAGCG |
| | | AATGCCTCCTGTCAG |
| | | ATTATACCTCCCCTC |
| | | TTGGTTCAACCGATG |
| | | CAGTTCTCCAACTCG |
| | | TCCTGTTTATCTTCC |
| | | CCTTTCATTAACGAT |
| | | ACGGAACAAATAGAC |
| | | TTAGGTGCAGTCACC |
| | | TTTACTAACTGCACC |
| | | TCTGTAGCCAATGTC |
| | | AGTAGTCCTTTATGT |
| | | GCCCTAAACGGGTCA |
| | | GTCTTCCTCTGTGGA |
| | | AATAACATGGCATAC |
| | | ACCTATTTACCCCAA |
| | | AACTGGACAGGACTT |
| | | TGCGTCCAAGCCTCC |
| | | CTCCTCCCCGACATT |
| | | GACATCATCCCGGGG |
| | | GATGAGCCAGTCCCC |
| | | ATTCCTGCCATTGAT |
| | | CATTATATACATAGA |
| | | CCTAAACGAGCTGTA |
| | | CAGTTCATCCCTTTA |
| | | CTAGCTGGACTGGGA |
| | | ATCACCGCAGCATTC |
| | | ACCACCGGAGCTACA |
| | | GGCCTAGGTGTCTCC |
| | | GTCACCCAGTATACA |
| | | AAATTATCCCATCAG |
| | | TTAATATCTGATGTC |
| | | CAAGTCTTATCCGGT |
| | | ACCATACAAGATTTA |
| | | CAAGACCAGGTAGAC |
| | | TCGTTAGCTGAAGTA |
| | | GTTCTCCAAAATAGG |
| | | AGGGGACTGGACCTA |
| | | CTAACGGCAGAACAA |
| | | GGAGGAATTTGTTTA |
| | | GCCTTACAAGAAAAA |
| | | TGCTGTTTTTATGCT |
| | | AACAAGTCAGGAATT |
| | | GTGAGAAACAAAATA |
| | | AGAACCCTACAAGAA |
| | | GAATTACAAAAACGC |
| | | AGGGAAAGCCTGGCA |
| | | TCCAACCCTCTCTGG |
| | | ACCGGGCTGCAGGGC |
| | | TTTCTTCCGTACCTC |
| | | CTACCTCTCCTGGGA |
| | | CCCCTACTCACCCTC |
| | | CTACTCATACTAACC |
| | | ATTGGGCCATGCGTT |
| | | TTCAATCGATTGGTC |
| | | CAATTTGTTAAAGAC |
| | | AGGATCTCAGTGGTC |
| | | CAGGCTCTGGTTTTG |
| | | ACTCAGCAATATCAC |
| | | CAGCTAAAACCCATA |
| | | GAGTACGAGCCATGA |
| 53 | GALV | ATGCTTCTCACCTCA |
| | Envelope | AGCCCGCACCACCTT |
| | | CGGCACCAGATGAGT |
| | | CCTGGGAGCTGGAAA |
| | | AGACTGATCATCCTC |
| | | TTAAGCTGCGTATTC |
| | | GGAGACGGCAAAACG |
| | | AGTCTGCAGAATAAG |
| | | AACCCCCACCAGCCT |
| | | GTGACCCTCACCTGG |
| | | CAGGTACTGTCCCAA |
| | | ACTGGGGACGTTGTC |
| | | TGGGACAAAAAGGCA |
| | | GTCCAGCCCCTTTGG |
| | | ACTTGGTGGCCCTCT |
| | | CTTACACCTGATGTA |
| | | TGTGCCCTGGCGGCC |
| | | GGTCTTGAGTCCTGG |
| | | GATATCCCGGGATCC |
| | | GATGTATCGTCCTCT |
| | | AAAAGAGTTAGACCT |
| | | CCTGATTCAGACTAT |
| | | ACTGCCGCTTATAAG |
| | | CAAATCACCTGGGGA |
| | | GCCATAGGGTGCAGC |
| | | TACCCTCGGGCTAGG |
| | | ACCAGGATGGCAAAT |
| | | TCCCCCTTCTACGTG |
| | | TGTCCCCGAGCTGGC |
| | | CGAACCCATTCAGAA |
| | | GCTAGGAGGTGTGGG |
| | | GGGCTAGAATCCCTA |
| | | TACTGTAAAGAATGG |
| | | AGTTGTGAGACCACG |
| | | GGTACCGTTTATTGG |
| | | CAACCCAAGTCCTCA |
| | | TGGGACCTCATAACT |
| | | GTAAAATGGGACCAA |
| | | AATGTGAAATGGGAG |
| | | CAAAAATTTCAAAAG |
| | | TGTGAACAAACCGGC |
| | | TGGTGTAACCCCCTC |
| | | AAGATAGACTTCACA |
| | | GAAAAAGGAAAACTC |
| | | TCCAGAGATTGGATA |
| | | ACGGAAAAAACCTGG |
| | | GAATTAAGGTTCTAT |
| | | GTATATGGACACCCA |
| | | GGCATACAGTTGACT |
| | | ATCCGCTTAGAGGTC |
| | | ACTAACATGCCGGTT |
| | | GTGGCAGTGGGCCCA |
| | | GACCCTGTCCTTGCG |
| | | GAACAGGGACCTCCT |
| | | AGCAAGCCCCTCACT |
| | | CTCCCTCTCTCCCCA |
| | | CGGAAAGCGCCGCCC |
| | | ACCCCTCTACCCCCG |
| | | GCGGCTAGTGAGCAA |
| | | ACCCCTGCGGTGCAT |
| | | GGAGAAACTGTTACC |
| | | CTAAACTCTCCGCCT |
| | | CCCACCAGTGGCGAC |
| | | CGACTCTTTGGCCTT |
| | | GTGCAGGGGGCCTTC |
| | | CTAACCTTGAATGCT |
| | | ACCAACCCAGGGGCC |
| | | ACTAAGTCTTGCTGG |
| | | CTCTGTTTGGGCATG |
| | | AGCCCCCCTTATTAT |
| | | GAAGGGATAGCCTCT |
| | | TCAGGAGAGGTCGCT |
| | | TATACCTCCAACCAT |
| | | ACCCGATGCCACTGG |
| | | GGGGCCCAAGGAAAG |
| | | CTTACCCTCACTGAG |
| | | GTCTCCGGACTCGGG |
| | | TCATGCATAGGGAAG |
| | | GTGCCTCTTACCCAT |
| | | CAACATCTTTGCAAC |
| | | CAGACCTTACCCATC |
| | | AATTCCTCTAAAAAC |
| | | CATCAGTATCTGCTC |
| | | CCCTCAAACCATAGC |
| | | TGGTGGGCCTGCAGC |
| | | ACTGGCCTCACCCCC |
| | | TGCCTCTCCACCTCA |
| | | GTTTTTAATCAGTCT |
| | | AAAGACTTCTGTGTC |
| | | CAGGTCCAGCTGATC |
| | | CCCCGCATCTATTAC |
| | | CATTCTGAAGAAACC |
| | | TTGTTACAAGCCTAT |
| | | GACAAATCACCCCCC |
| | | AGGTTTAAAAGAGAG |
| | | CCTGCCTCACTTACC |
| | | CTAGCTGTCTTCCTG |
| | | GGGTTAGGGATTGCG |
| | | GCAGGTATAGGTACT |
| | | GGCTCAACCGCCCTA |
| | | ATTAAAGGGCCCATA |
| | | GACCTCCAGCAAGGC |
| | | CTAACCAGCCTCCAA |
| | | ATCGCCATTGACGCT |
| | | GACCTCCGGGCCCTT |
| | | CAGGACTCAATCAGC |
| | | AAGCTAGAGGACTCA |
| | | CTGACTTCCCTATCT |
| | | GAGGTAGTACTCCAA |
| | | AATAGGAGAGGCCTT |
| | | GACTTACTATTCCTT |
| | | AAAGAAGGAGGCCTC |
| | | TGCGCGGCCCTAAAA |
| | | GAAGAGTGCTGTTTT |
| | | TATGTAGACCACTCA |
| | | GGTGCAGTACGAGAC |
| | | TCCATGAAAAAACTT |
| | | AAAGAAAGACTAGAT |
| | | AAAAGACAGTTAGAG |
| | | CGCCAGAAAAACCAA |
| | | AACTGGTATGAAGGG |
| | | TGGTTCAATAACTCC |
| | | CCTTGGTTTACTACC |
| | | CTACTATCAACCATC |
| | | GCTGGGCCCCTATTG |
| | | CTCCTCCTTTTGTTA |
| | | CTCACTCTTGGGCCC |
| | | TGCATCATCAATAAA |
| | | TTAATCCAATTCATC |
| | | AATGATAGGATAAGT |
| | | GCAGTCAAAATTTTA |
| | | GTCCTTAGACAGAAA |
| | | TATCAGACCCTAGAT |
| | | AACGAGGAAAACCTT |
| | | TAA |
| 54 | FUG | ATGGTTCCGCAGGTT |
| | Envelope | CTTTTGTTTGTACTC |
| | | CTTCTGGGTTTTTCG |
| | | TTGTGTTTCGGGAAG |
| | | TTCCCCATTTACACG |
| | | ATACCAGACGAACTT |
| | | GGTCCCTGGAGCCCT |
| | | ATTGACATACACCAT |
| | | CTCAGCTGTCCAAAT |
| | | AACCTGGTTGTGGAG |
| | | GATGAAGGATGTACC |
| | | AACCTGTCCGAGTTC |
| | | TCCTACATGGAACTC |
| | | AAAGTGGGATACATC |
| | | TCAGCCATCAAAGTG |
| | | AACGGGTTCACTTGC |
| | | ACAGGTGTTGTGACA |
| | | GAGGCAGAGACCTAC |
| | | ACCAACTTTGTTGGT |
| | | TATGTCACAACCACA |
| | | TTCAAGAGAAAGCAT |
| | | TTCCGCCCCACCCCA |
| | | GACGCATGTAGAGCC |
| | | GCGTATAACTGGAAG |
| | | ATGGCCGGTGACCCC |
| | | AGATATGAAGAGTCC |
| | | CTACACAATCCATAC |
| | | CCCGACTACCACTGG |
| | | CTTCGAACTGTAAGA |
| | | ACCACCAAAGAGTCC |
| | | CTCATTATCATATCC |
| | | CCAAGTGTGACAGAT |
| | | TTGGACCCATATGAC |
| | | AAATCCCTTCACTCA |
| | | AGGGTCTTCCCTGGC |
| | | GGAAAGTGCTCAGGA |
| | | ATAACGGTGTCCTCT |
| | | ACCTACTGCTCAACT |
| | | AACCATGATTACACC |
| | | ATTTGGATGCCCGAG |
| | | AATCCGAGACCAAGG |
| | | ACACCTTGTGACATT |
| | | TTTACCAATAGCAGA |
| | | GGGAAGAGAGCATCC |
| | | AACGGGAACAAGACT |
| | | TGCGGCTTTGTGGAT |
| | | GAAAGAGGCCTGTAT |
| | | AAGTCTCTAAAAGGA |
| | | GCATGCAGGCTCAAG |
| | | TTATGTGGAGTTCTT |
| | | GGACTTAGACTTATG |
| | | GATGGAACATGGGTC |
| | | GCGATGCAAACATCA |
| | | GATGAGACCAAATGG |
| | | TGCCCTCCAGATCAG |
| | | TTGGTGAATTTGCAC |
| | | GACTTTCGCTCAGAC |
| | | GAGATCGAGCATCTC |
| | | GTTGTGGAGGAGTTA |
| | | GTTAAGAAAAGAGAG |
| | | GAATGTCTGGATGCA |
| | | TTAGAGTCCATCATG |
| | | ACCACCAAGTCAGTA |
| | | AGTTTCAGACGTCTC |
| | | AGTCACCTGAGAAAA |
| | | CTTGTCCCAGGGTTT |
| | | GGAAAAGCATATACC |
| | | ATATTCAACAAAACC |
| | | TTGATGGAGGCTGAT |
| | | GCTCACTACAAGTCA |
| | | GTCCGGACCTGGAAT |
| | | GAGATCATCCCCTCA |
| | | AAAGGGTGTTTGAAA |
| | | GTTGGAGGAAGGTGC |
| | | CATCCTCATGTGAAC |
| | | GGGGTGTTTTTCAAT |
| | | GGTATAATATTAGGG |
| | | CCTGACGACCATGTC |
| | | CTAATCCCAGAGATG |
| | | CAATCATCCCTCCTC |
| | | CAGCAACATATGGAG |
| | | TTGTTGGAATCTTCA |
| | | GTTATCCCCCTGATG |
| | | CACCCCCTGGCAGAC |
| | | CCTTCTACAGTTTTC |
| | | AAAGAAGGTGATGAG |
| | | GCTGAGGATTTTGTT |
| | | GAAGTTCACCTCCCC |
| | | GATGTGTACAAACAG |
| | | ATCTCAGGGGTTGAC |
| | | CTGGGTCTCCCGAAC |
| | | TGGGGAAAGTATGTA |
| | | TTGATGACTGCAGGG |
| | | GCCATGATTGGCCTG |
| | | GTGTTGATATTTTCC |
| | | CTAATGACATGGTGC |
| | | AGAGTTGGTATCCAT |
| | | CTTTGCATTAAATTA |
| | | AAGCACACCAAGAAA |
| | | AGACAGATTTATACA |
| | | GACATAGAGATGAAC |
| | | CGACTTGGAAAGTAA |
| 55 | LCMV | ATGGGTCAGATTGTG |
| | Envelope | ACAATGTTTGAGGCT |
| | | CTGCCTCACATCATC |
| | | GATGAGGTGATCAAC |
| | | ATTGTCATTATTGTG |
| | | CTTATCGTGATCACG |
| | | GGTATCAAGGCTGTC |
| | | TACAATTTTGCCACC |
| | | TGTGGGATATTCGCA |
| | | TTGATCAGTTTCCTA |
| | | CTTCTGGCTGGCAGG |
| | | TCCTGTGGCATGTAC |
| | | GGTCTTAAGGGACCC |
| | | GACATTTACAAAGGA |
| | | GTTTACCAATTTAAG |
| | | TCAGTGGAGTTTGAT |
| | | ATGTCACATCTGAAC |
| | | CTGACCATGCCCAAC |
| | | GCATGTTCAGCCAAC |
| | | AACTCCCACCATTAC |
| | | ATCAGTATGGGGACT |
| | | TCTGGACTAGAATTG |
| | | ACCTTCACCAATGAT |
| | | TCCATCATCAGTCAC |
| | | AACTTTTGCAATCTG |
| | | ACCTCTGCCTTCAAC |
| | | AAAAAGACCTTTGAC |
| | | CACACACTCATGAGT |
| | | ATAGTTTCGAGCCTA |
| | | CACCTCAGTATCAGA |
| | | GGGAACTCCAACTAT |
| | | AAGGCAGTATCCTGC |
| | | GACTTCAACAATGGC |
| | | ATAACCATCCAATAC |
| | | AACTTGACATTCTCA |
| | | GATCGACAAAGTGCT |
| | | CAGAGCCAGTGTAGA |
| | | ACCTTCAGAGGTAGA |
| | | GTCCTAGATATGTTT |
| | | AGAACTGCCTTCGGG |
| | | GGGAAATACATGAGG |
| | | AGTGGCTGGGGCTGG |
| | | ACAGGCTCAGATGGC |
| | | AAGACCACCTGGTGT |
| | | AGCCAGACGAGTTAC |
| | | CAATACCTGATTATA |
| | | CAAAATAGAACCTGG |
| | | GAAAACCACTGCACA |
| | | TATGCAGGTCCTTTT |
| | | GGGATGTCCAGGATT |
| | | CTCCTTTCCCAAGAG |
| | | AAGACTAAGTTCTTC |
| | | ACTAGGAGACTAGCG |
| | | GGCACATTCACCTGG |
| | | ACTTTGTCAGACTCT |
| | | TCAGGGGTGGAGAAT |
| | | CCAGGTGGTTATTGC |
| | | CTGACCAAATGGATG |
| | | ATTCTTGCTGCAGAG |
| | | CTTAAGTGTTTCGGG |
| | | AACACAGCAGTTGCG |
| | | AAATGCAATGTAAAT |
| | | CATGATGCCGAATTC |
| | | TGTGACATGCTGCGA |
| | | CTAATTGACTACAAC |
| | | AAGGCTGCTTTGAGT |
| | | AAGTTCAAAGAGGAC |
| | | GTAGAATCTGCCTTG |
| | | CACTTATTCAAAACA |
| | | ACAGTGAATTCTTTG |
| | | ATTTCAGATCAACTA |
| | | CTGATGAGGAACCAC |
| | | TTGAGAGATCTGATG |
| | | GGGGTGCCATATTGC |
| | | AATTACTCAAAGTTT |
| | | TGGTACCTAGAACAT |
| | | GCAAAGACCGGCGAA |
| | | ACTAGTGTCCCCAAG |
| | | TGCTGGCTTGTCACC |
| | | AATGGTTCTTACTTA |
| | | AATGAGACCCACTTC |
| | | AGTGATCAAATCGAA |
| | | CAGGAAGCCGATAAC |
| | | ATGATTACAGAGATG |
| | | TTGAGGAAGGATTAC |
| | | ATAAAGAGGCAGGGG |
| | | AGTACCCCCCTAGCA |
| | | TTGATGGACCTTCTG |
| | | ATGTTTTCCACATCT |
| | | GCATATCTAGTCAGC |
| | | ATCTTCCTGCACCTT |
| | | GTCAAAATACCAACA |
| | | CACAGGCACATAAAA |
| | | GGTGGCTCATGTCCA |
| | | AAGCCACACCGATTA |
| | | ACCAACAAAGGAATT |
| | | TGTAGTTGTGGTGCA |
| | | TTTAAGGTGCCTGGT |
| | | GTAAAAACCGTCTGG |
| | | AAAAGACGCTGA |
| 56 | FPV Envelope | ATGAACACTCAAATC |
| | | CTGGTTTTCGCCCTT |
| | | GTGGCAGTCATCCCC |
| | | ACAAATGCAGACAAA |
| | | ATTTGTCTTGGACAT |
| | | CATGCTGTATCAAAT |
| | | GGCACCAAAGTAAAC |
| | | ACACTCACTGAGAGA |
| | | GGAGTAGAAGTTGTC |
| | | AATGCAACGGAAACA |
| | | GTGGAGCGGACAAAC |
| | | ATCCCCAAAATTTGC |
| | | TCAAAAGGGAAAAGA |
| | | ACCACTGATCTTGGC |
| | | CAATGCGGACTGTTA |
| | | GGGACCA |
| | | TTACCGGACCACCTC |
| | | AATGCGACCAATTTC |
| | | TAGAATTTTCAGCTG |
| | | ATCTAATAATCGAGA |
| | | GACGAGAAGGAAATG |
| | | ATGTTTGTTACCCGG |
| | | GGAAGTTTGTTAATG |
| | | AAGAGGCATTGCGAC |
| | | AAATCCTCAGAGGAT |
| | | CAGGTGGGATTGACA |
| | | AAGAAACAATGGGAT |
| | | TCACATATAGTGGAA |
| | | TAAGGACCAACGGAA |
| | | CAACTAGTGCATGTA |
| | | GAAGATCAGGGTCTT |
| | | CATTCTATGCAGAAA |
| | | TGGAGTGGCTCCTGT |
| | | CAAATACAGACAATG |
| | | CTGCTTTCCCACAAA |
| | | TGACAAAATCATACA |
| | | AAAACACAAGGAGAG |
| | | AATCAGCTCTGATAG |
| | | TCTGGGGAATCCACC |
| | | ATTCAGGATCAACCA |
| | | CCGAACAGACCAAAC |
| | | TATATGGGAGTGGAA |
| | | ATAAACTGATAACAG |
| | | TCGGGAGTTCCAAAT |
| | | ATCATCAATCTTTTG |
| | | TGCCGAGTCCAGGAA |
| | | CACGACCGCAGATAA |
| | | ATGGCCAGTCCGGAC |
| | | GGATTGATTTTCATT |
| | | GGTTGATCTTGGATC |
| | | CCAATGATACAGTTA |
| | | CTTTTAGTTTCAATG |
| | | GGGCTTTCATAGCTC |
| | | CAAATCGTGCCAGCT |
| | | TCTTGAGGGGAAAGT |
| | | CCATGGGGATCCAGA |
| | | GCGATGTGCAGGTTG |
| | | ATGCCAATTGCGAAG |
| | | GGGAATGCTACCACA |
| | | GTGGAGGGACTATAA |
| | | CAAGCAGATTGCCTT |
| | | TTCAAAACATCAATA |
| | | GCAGAGCAGTTGGCA |
| | | AATGCCCAAGATATG |
| | | TAAAACAGGAAAGTT |
| | | TATTATTGGCAACTG |
| | | GGATGAAGAACGTTC |
| | | CCGAACCTTCCAAAA |
| | | AAAGGAAAAAAAGAG |
| | | GCCTGTTTGGCGCTA |
| | | TAGCAGGGTTTATTG |
| | | AAAATGGTTGGGAAG |
| | | GTCTGGTCGACGGGT |
| | | GGTACGGTTTCAGGC |
| | | ATCAGAATGCACAAG |
| | | GAGAAGGAACTGCAG |
| | | CAGACTACAAAAGCA |
| | | CCCAATCGGCAATTG |
| | | ATCAGATAACCGGAA |
| | | AGTTAAATAGACTCA |
| | | TTGAGAAAACCAACC |
| | | AGCAATTTGAGCTAA |
| | | TAGATAATGAATTCA |
| | | CTGAGGTGGAAAAGC |
| | | AGATTGGCAATTTAA |
| | | TTAACTGGACCAAAG |
| | | ACTCCATCACAGAAG |
| | | TATGGTCTTACAATG |
| | | CTGAACTTCTTGTGG |
| | | CAATGGAAAACCAGC |
| | | ACACTATTGATTTGG |
| | | CTGATTCAGAGATGA |
| | | ACAAGCTGTATGAGC |
| | | GAGTGAGGAAACAAT |
| | | TAAGGGAAAATGCTG |
| | | AAGAGGATGGCACTG |
| | | GTTGCTTTGAAATTT |
| | | TTCATAAATGTGACG |
| | | ATGATTGTATGGCTA |
| | | GTATAAGGAACAATA |
| | | CTTATGATCACAGCA |
| | | AATACAGAGAAGAAG |
| | | CGATGCAAAATAGAA |
| | | TACAAATTGACCCAG |
| | | TCAAATTGAGTAGTG |
| | | GCTACAAAGATGTGA |
| | | TACTTTGGTTTAGCT |
| | | TCGGGGCATCATGCT |
| | | TTTTGCTTCTTGCCA |
| | | TTGCAATGGGCCTTG |
| | | TTTTCATATGTGTGA |
| | | AGAACGGAAACATGC |
| | | GGTGCACTATTTGTA |
| | | TATAA |
| 57 | RRV | AGTGTAACAGAGCAC |
| | Envelope | TTTAATGTGTATAAG |
| | | GCTACTAGACCATAC |
| | | CTAGCACATTGCGCC |
| | | GATTGCGGGGACGGG |
| | | TACTTCTGCTATAGC |
| | | CCAGTTGCTATCGAG |
| | | GAGATCCGAGATGAG |
| | | GCGTCTGATGGCATG |
| | | CTTAAGATCCAAGTC |
| | | TCCGCCCAAATAGGT |
| | | CTGGACAAGGCAGGC |
| | | ACCCACGCCCACACG |
| | | AAGCTCCGATATATG |
| | | GCTGGTCATGATGTT |
| | | CAGGAATCTAAGAGA |
| | | GATTCCTTGAGGGTG |
| | | TACACGTCCGCAGCG |
| | | TGCTCCATACATGGG |
| | | ACGATGGGACACTTC |
| | | ATCGTCGCACACTGT |
| | | CCACCAGGCGACTAC |
| | | CTCAAGGTTTCGTTC |
| | | GAGGACGCAGATTCG |
| | | CACGTGAAGGCATGT |
| | | AAGGTCCAATACAAG |
| | | CACAATCCATTGCCG |
| | | GTGGGTAGAGAGAAG |
| | | TTCGTGGTTAGACCA |
| | | CACTTTGGCGTAGAG |
| | | CTGCCATGCACCTCA |
| | | TACCAGCTGACAACG |
| | | GCTCCCACCGACGAG |
| | | GAGATTGACATGCAT |
| | | ACACCGCCAGATATA |
| | | CCGGATCGCACCCTG |
| | | CTATCACAGACGGCG |
| | | GGCAACGTCAAAATA |
| | | ACAGCAGGCGGCAGG |
| | | ACTATCAGGTACAAC |
| | | TGTACCTGCGGCCGT |
| | | GACAACGTAGGCACT |
| | | ACCAGTACTGACAAG |
| | | ACCATCAACACATGC |
| | | AAGATTGACCAATGC |
| | | CATGCTGCCGTCACC |
| | | AGCCATGACAAATGG |
| | | CAATTTACCTCTCCA |
| | | TTTGTTCCCAGGGCT |
| | | GATCAGACAGCTAGG |
| | | AAAGGCAAGGTACAC |
| | | GTTCCGTTCCCTCTG |
| | | ACTAACGTCACCTGC |
| | | CGAGTGCCGTTGGCT |
| | | CGAGCGCCGGATGCC |
| | | ACCTATGGTAAGAAG |
| | | GAGGTGACCCTGAGA |
| | | TTACACCCAGATCAT |
| | | CCGACGCTCTTCTCC |
| | | TATAGGAGTTTAGGA |
| | | GCCGAACCGCACCCG |
| | | TACGAGGAATGGGTT |
| | | GACAAGTTCTCTGAG |
| | | CGCATCATCCCAGTG |
| | | ACGGAAGAAGGGATT |
| | | GAGTACCAGTGGGGC |
| | | AACAACCCGCCGGTC |
| | | TGCCTGTGGGCGCAA |
| | | CTGACGACCGAGGGC |
| | | AAACCCCATGGCTGG |
| | | CCACATGAAATCATT |
| | | CAGTACTATTATGGA |
| | | CTATACCCCGCCGCC |
| | | ACTATTGCCGCAGTA |
| | | TCCGGGGCGAGTCTG |
| | | ATGGCCCTCCTAACT |
| | | CTGGCGGCCACATGC |
| | | TGCATGCTGGCCACC |
| | | GCGAGGAGAAAGTGC |
| | | CTAACACCGTACGCC |
| | | CTGACGCCAGGAGCG |
| | | GTGGTACCGTTGACA |
| | | CTGGGGCTGCTTTGC |
| | | TGCGCACCGAGGGCG |
| | | AATGCA |
| 58 | MLV 10A1 | ATGGAAGGTCCAGCG |
| | Envelope | TTCTCAAAACCCCTT |
| | | AAAGATAAGATTAAC |
| | | CCGTGGAAGTCCTTA |
| | | ATGGTCATGGGGGTC |
| | | TATTTAAGAGTAGGG |
| | | ATGGCAGAGAGCCCC |
| | | CATCAGGTCTTTAAT |
| | | GTAACCTGGAGAGTC |
| | | ACCAACCTGATGACT |
| | | GGGCGTACCGCCAAT |
| | | GCCACCTCCCTTTTA |
| | | GGAACTGTACAAGAT |
| | | GCCTTCCCAAGATTA |
| | | TATTTTGATCTATGT |
| | | GATCTGGTCGGAGAA |
| | | GAGTGGGACCCTTCA |
| | | GACCAGGAACCATAT |
| | | GTCGGGTATGGCTGC |
| | | AAATACCCCGGAGGG |
| | | AGAAAGCGGACCCGG |
| | | ACTTTTGACTTTTAC |
| | | GTGTGCCCTGGGCAT |
| | | ACCGTAAAATCGGGG |
| | | TGTGGGGGGCCAAGA |
| | | GAGGGCTACTGTGGT |
| | | GAATGGGGTTGTGAA |
| | | ACCACCGGACAGGCT |
| | | TACTGGAAGCCCACA |
| | | TCATCATGGGACCTA |
| | | ATCTCCCTTAAGCGC |
| | | GGTAACACCCCCTGG |
| | | GACACGGGATGCTCC |
| | | AAAATGGCTTGTGGC |
| | | CCCTGCTACGACCTC |
| | | TCCAAAGTATCCAAT |
| | | TCCTTCCAAGGGGCT |
| | | ACTCGAGGGGGCAGA |
| | | TGCAACCCTCTAGTC |
| | | CTAGAATTCACTGAT |
| | | GCAGGAAAAAAGGCT |
| | | AATTGGGACGGGCCC |
| | | AAATCGTGGGGACTG |
| | | AGACTGTACCGGACA |
| | | GGAACAGATCCTATT |
| | | ACCATGTTCTCCCTG |
| | | ACCCGCCAGGTCCTC |
| | | AATATAGGGCCCCGC |
| | | ATCCCCATTGGGC |
| | | CTAATCCCGTGATCA |
| | | CTGGTCAACTACCCC |
| | | CCTCCCGACCCGTGC |
| | | AGATCAGGCTCCCCA |
| | | GGCCTCCTCAGCCTC |
| | | CTCCTACAGGCGCAG |
| | | CCTCTATAGTCCCTG |
| | | AGACTGCCCCACCTT |
| | | CTCAACAACCTGGGA |
| | | CGGGAGACAGGCTGC |
| | | TAAACCTGGTAGAAG |
| | | GAGCCTATCAGGCGC |
| | | TTAACCTCACCAATC |
| | | CCGACAAGACCCAAG |
| | | AATGTTGGCTGTGCT |
| | | TAGTGTCGGGACCTC |
| | | CTTATTACGAAGGAG |
| | | TAGCGGTCGTGGGCA |
| | | CTTATACCAATCATT |
| | | CTACCGCCCCGGCCA |
| | | GCTGTACGGCCACTT |
| | | CCCAACATAAGCTTA |
| | | CCCTATCTGAAGTGA |
| | | CAGGACAGGGC |
| | | CTATGCATGGGAGCA |
| | | CTACCTAAAACTCAC |
| | | CAGGCCTTATGTAAC |
| | | ACCACCCAAAGTGCC |
| | | GGCTCAGGATCCTAC |
| | | TACCTTGCAGCACCC |
| | | GCTGGAACAATGTGG |
| | | GCTTGTAGCACTGGA |
| | | TTGACTCCCTGCTTG |
| | | TCCACCACGATGCTC |
| | | AATCTAACCACAGAC |
| | | TATTGTGTATTAGTT |
| | | GAGCTCTGGCCCAGA |
| | | ATAATTTACCACTCC |
| | | CCCGATTATATGTAT |
| | | GGTCAGCTTGAACAG |
| | | CGTACCAAATATAAG |
| | | AGGGAGCCAGTATCG |
| | | TTGACCCTGGCCCTT |
| | | CTGCTAGGAGGATTA |
| | | ACCATGGGAGGGATT |
| | | GCAGCTGGAATAGGG |
| | | ACGGGGACCACTGCC |
| | | CTAATCAAAACCCAG |
| | | CAGTTTGAGCAGCTT |
| | | CACGCCGCTATCCAG |
| | | ACAGACCTCAACGAA |
| | | GTCGAAAAATCAATT |
| | | ACCAACCTAGAAAAG |
| | | TCACTGACCTCGTTG |
| | | TCTGAAGTAGTCCTA |
| | | CAGAACCGAAGAGGC |
| | | CTAGATTTGCTCTTC |
| | | CTAAAAGAGGGAGGT |
| | | CTCTGCGCAGCCCTA |
| | | AAAGAAGAATGTTGT |
| | | TTTTATGCAGACCAC |
| | | ACGGGACTAGTGAGA |
| | | GACAGCATGGCCAAA |
| | | CTAAGGGAAAGGCTT |
| | | AATCAGAGACAAAAA |
| | | CTATTTGAGTCAGGC |
| | | CAAGGTTGGTTCGAA |
| | | GGGCAGTTTAATAGA |
| | | TCCCCCTGGTTTACC |
| | | ACCTTAATCTCCACC |
| | | ATCATGGGACCTCTA |
| | | ATAGTACTCTTACTG |
| | | ATCTTACTCTTTGGA |
| | | CCCTGCATTCTCAAT |
| | | CGATTGGTCCAATTT |
| | | GTTAAAGACAGGATC |
| | | TCAGTGGTCCAGGCT |
| | | CTGGTTTTGACTCAA |
| | | CAATATCACCAGCTA |
| | | AAACCTATAGAGTAC |
| | | GAGCCATGA |
| 59 | EboV | ATGGGTGTTACAGGA |
| | Envelope | ATATTGCAGTTACCT |
| | | CGTGATCGATTCAAG |
| | | AGGACATCATTCTTT |
| | | CTTTGGGTAATTATC |
| | | CTTTTCCAAAGAACA |
| | | TTTTCCATCCCACTT |
| | | GGAGTCATCCACAAT |
| | | AGCACATTACAGGTT |
| | | AGTGATGTCGACAAA |
| | | CTGGTTTGCCGTGAC |
| | | AAACTGTCATCCACA |
| | | AATCAATTGAGATCA |
| | | GTTGGACTGAATCTC |
| | | GAAGGGAATGGAGTG |
| | | GCAACTGACGTGCCA |
| | | TCTGCAACTAAAAGA |
| | | TGGGGCTTCAGGTCC |
| | | GGTGTCCCACCAAAG |
| | | GTGGTCAATTATGAA |
| | | GCTGGTGAATGGGCT |
| | | GAAAACTGCTACAAT |
| | | CTTGAAATCAAAAAA |
| | | CCTGACGGGAGTGAG |
| | | TGTCTACCAGCAGCG |
| | | CCAGACGGGATTCGG |
| | | GGCTTCCCCCGGTGC |
| | | CGGTATGTGCACAAA |
| | | GTATCAGGAACGGGA |
| | | CCGTGTGCCGGAGAC |
| | | TTTGCCTTCCACAAA |
| | | GAGGGTGCTTTCTTC |
| | | CTGTATGACCGACTT |
| | | GCTTCCACAGTTATC |
| | | TACCGAGGAACGACT |
| | | TTCGCTGAAGGTGTC |
| | | GTTGCATTTCTGATA |
| | | CTGCCCCAAGCTAAG |
| | | AAGGACTTCTTCAGC |
| | | TCACACCCCTTGAGA |
| | | GAGCCGGTCAATGCA |
| | | ACGGAGGACCCGTCT |
| | | AGTGGCTACTATTCT |
| | | ACCACAATTAGATAT |
| | | CAAGCTACCGGTTTT |
| | | GGAACCAATGAGACA |
| | | GAGTATTTGTTCGAG |
| | | GTTGACAATTTGACC |
| | | TACGTCCAACTTGAA |
| | | TCAAGATTCACACCA |
| | | CAGTTTCTGCTCCAG |
| | | CTGAATGAGACAATA |
| | | TATACAAGTGGGAAA |
| | | AGGAGCAATACCACG |
| | | GGAAAACTAATTTGG |
| | | AAGGTCAACCCCGAA |
| | | ATTGATACAACAATC |
| | | GGGGAGTGGGCCTTC |
| | | TGGGAAACTAAAAAA |
| | | ACCTCACTAGAAAAA |
| | | TTCGCAGTGAAGAGT |
| | | TGTCTTTCACAGCTG |
| | | TATCAAACAGAGCCA |
| | | AAAACATCAGTGGTC |
| | | AGAGTCCGGCGCGAA |
| | | CTTCTTCCGACCCAG |
| | | GGACCAACACAACAA |
| | | CTGAAGACCACAAAA |
| | | TCATGGCTTCAGAAA |
| | | ATTCCTCTGCAATGG |
| | | TTCAAGTGCACAGTC |
| | | AAGGAAGGGAAGCTG |
| | | CAGTGTCGCATCTGA |
| | | CAACCCTTGCCACAA |
| | | TCTCCACGAGTCCTC |
| | | AACCCCCCACAACCA |
| | | AACCAGGTCCGGACA |
| | | ACAGCACCCACAATA |
| | | CACCCGTGTATAAAC |
| | | TTGACATCTCTGAGG |
| | | CAACTCAAGTTGAAC |
| | | AACATCACCGCAGAA |
| | | CAGACAACGACAGCA |
| | | CAGCCTCCGACACTC |
| | | CCCCCGCCACGACCG |
| | | CAGCCGGACCCCTAA |
| | | AAGCAGAGAACACCA |
| | | ACACGAGCAAGGGTA |
| | | CCGACCTCCTGGACC |
| | | CCGCCACCACAACAA |
| | | GTCCCCAAAACCACA |
| | | GCGAGACCGCTGGCA |
| | | ACAACAACACTCATC |
| | | ACCAAGATACCGGAG |
| | | AAGAGAGTGCCAGCA |
| | | GCGGGAAGCTAGGCT |
| | | TAATTACCAATACTA |
| | | TTGCTGGAGTCGCAG |
| | | GACTGATCACAGGCG |
| | | GGAGGAGAGCTCGAA |
| | | GAGAAGCAATTGTCA |
| | | ATGCTCAACCCAAAT |
| | | GCAACCCTAATTTAC |
| | | ATTACTGGACTACTC |
| | | AGGATGAAGGTGCTG |
| | | CAATCGGACTGGCCT |
| | | GGATACCATATTTCG |
| | | GGCCAGCAGCCGAGG |
| | | GAATTTACATAGAGG |
| | | GGCTGATGCACAATC |
| | | AAGATGGTTTAATCT |
| | | GTGGGTTGAGACAGC |
| | | TGGCCAACGAGACGA |
| | | CTCAAGCTCTTCAAC |
| | | TGTTCCTGAGAGCCA |
| | | CAACCGAGCTACGCA |
| | | CCTTTTCAATCCTCA |
| | | ACCGTAAGGCAATTG |
| | | ATTTCTTGCTGCAGC |
| | | GATGGGGCGGCACAT |
| | | GCCACATTTTGGGAC |
| | | CGGACTGCTGTATCG |
| | | AACCACATGATTGGA |
| | | CCAAGAACATAACAG |
| | | ACAAAATTGATCAGA |
| | | TTATTCATGATTTTG |
| | | TTGATAAAACCCTTC |
| | | CGGACCAGGGGGACA |
| | | ATGACAATTGGTGGA |
| | | CAGGATGGAGACAAT |
| | | GGATACCGGCAGGTA |
| | | TTGGAGTTACAGGCG |
| | | TTATAATTGCAGTTA |
| | | TCGCTTTATTCTGTA |
| | | TATGCAAATTTGTCT |
| | | TTTAG |
| 60 | Thyroxin | CTTTCTCTTTTGTTT |
| | binding | TACATGAAGGGTCTG |
| | globulin | GCAGCCAAAGCAATC |
| | promoter | ACTCAAAGTTCAAAC |
| | (TBG) | CTTATCATTTTTTGC |
| | | TTTGTTCCTCTTGGC |
| | | CTTGGTTTTGTACAT |
| | | CAGCTTTGAAAATAC |
| | | CATCCCAGGGTTAAT |
| | | GCTGGGGTTAATTTA |
| | | TAACTAAGAGTGCTC |
| | | TAGTTTTGCAATACA |
| | | GGACATGCTATAAAA |
| | | ATGGAAAGATGTTGC |
| | | TTTCTGAG |
| 61 | DNA | GCGAGAACTTGTGCC |
| | fragment | TCCCCGTGTTCCTGC |
| | containing | TCTTTGTCCCTCTGT |
| | prothrombin | CCTACTTAGACTAAT |
| | enhancer and | ATTTGCCTTGGGTAC |
| | human alpha-1 | TGCAAACAGGAAATG |
| | anti-trypsin | GGGGAGGGACAGGAG |
| | promoter | TAGGGCGGAGGGTAG |
| | | CCCGGGGATCTTGCT |
| | | ACCAGTGGAACAGCC |
| | | ACTAAGGATTCTGCA |
| | | GTGAGAGCAGAGGGC |
| | | CAGCTAAGTGGTACT |
| | | CTCCCAGAGACTGTC |
| | | TGACTCACGCCACCC |
| | | CCTCCACCTTGGACA |
| | | CAGGACGCTGTGGTT |
| | | TCTGAGCCAGGTACA |
| | | ATGACTCCTTTCGGT |
| | | AAGTGCAGTGGAAGC |
| | | TGTACACTGCCCAGG |
| | | CAAAGCGTCCGGGCA |
| | | GCGTAGGCGGGCGAC |
| | | TCAGATCCCAGCCAG |
| | | TGGACTTAGCCCCTG |
| | | TTTGCTCCTCCGATA |
| | | ACTGGGGTGACCTTG |
| | | GTTAATATTCACCAG |
| | | CAGCCTCCCCCGTTG |
| | | CCCCTCTGGATCCAC |
| | | TGCTTAAATACGGAC |
| | | GAGGACAGGGCCCTG |
| | | TCTCCTCAGCTTCAG |
| | | GCACCACCACTGACC |
| | | TGGGACAGTGAAT |
| 62 | DNA | GTTAATCATTAACGT |
| | fragment | TAATCATTAACGTTA |
| | containing | ATCATTAACGTTAAT |
| | prothrombin | CATTAACGTTAATCA |
| | enhancer, | TTAACATCGATGCGA |
| | human alpha-1 | GAACTTGTGCCTCCC |
| | anti-trypsin | CGTGTTCCTGCTCTT |
| | promoter, and | TGTCCCTCTGTCCTA |
| | five HNF1 | CTTAGACTAATATTT |
| | binding sites | GCCTTGGGTACTGCA |
| | | AACAGGAAATGGGGG |
| | | AGGGACAGGAGTAGG |
| | | GCGGAGGGTAGGATT |
| | | CTGCAGTGAGAGCAG |
| | | AGGGCCAGCTAAGTG |
| | | GTACTCTCCCAGAGA |
| | | CTGTCTGACTCACGC |
| | | CACCCCCTCCACCTT |
| | | GGACACAGGACGCTG |
| | | TGGTTTCTGAGCCAG |
| | | GTACAATGACTCCTT |
| | | TCGGTAAGTGCAGTG |
| | | GAAGCTGTACACTGC |
| | | CCAGGCAAAGCGTCC |
| | | GGGCAGCGTAGGCGG |
| | | GCGACTCAGATCCCA |
| | | GCCAGTGGACTTAGC |
| | | CCCTGTTTGCTCCTC |
| | | CGATAACTGGGGTGA |
| | | CCTTGGTTAATATTC |
| | | ACCAGCAGCCTCCCC |
| | | CGTTGCCCCTCTGGA |
| | | TCCACTGCTTAAATA |
| | | CGGACGAGGACAGGG |
| | | CCCTGTCTCCTCAGC |
| | | TTCAGGCACCACCAC |
| | | TGACCTGGGACAGTG |
| | | AAT |
| 63 | DNA | GTTAATCATTAACGC |
| | fragment | TTGTACTTTGGTACA |
| | containing | GTTAATCATTAACGC |
| | prothrombin | TTGTACTTTGGTACA |
| | enhancer, | GTTAATCATTAACGC |
| | human alpha-1 | TTGTACTTTGGTACA |
| | anti-trypsin | ATCGATGCGAGAACT |
| | promoter, and | TGTGCCTCCCCGTGT |
| | three | TCCTGCTCTTTGTCC |
| | HNF1/HNF4 | CTCTGTCCTACTTAG |
| | binding sites | ACTAATATTTGCCTT |
| | | GGGTACTGCAAACAG |
| | | GAAATGGGGGAGGGA |
| | | CAGGAGTAGGGCGGA |
| | | GGGTAGCCCGGGGAT |
| | | TCTGCAGTGAGAGCA |
| | | GAGGGCCAGCTAAGT |
| | | GGTACTCTCCCAGAG |
| | | ACTGTCTGACTCACG |
| | | CCACCCCCTCCACCT |
| | | TGGACACAGGACGCT |
| | | GTGGTTTCTGAGCCA |
| | | GGTACAATGACTCCT |
| | | TTCGGTAAGTGCAGT |
| | | GGAAGCTGTACACTG |
| | | CCCAGGCAAAGCGTC |
| | | CGGGCAGCGTAGGCG |
| | | GGCGACTCAGATCCC |
| | | AGCCAGTGGACTTAG |
| | | CCCCTGTTTGCTCCT |
| | | CCGATAACTGGGGTG |
| | | ACCTTGGTTAATATT |
| | | CACCAGCAGCCTCCC |
| | | CCGTTGCCCCTCTGG |
| | | ATCCACTGCTTAAAT |
| | | ACGGACGAGGACAGG |
| | | GCCCTGTCTCCTCAG |
| | | CTTCAGGCACCACCA |
| | | CTGACCTGGGACAGT |
| | | GAAT |
| 64 | hPAH FAM | TCGTGAAAGCTCATG |
| | TaqMan | GACAGTGGC |
| | Probe | |
| 65 | PAH TaqMan | AGATCTTGAGGCATG |
| | Forward | ACATTGG |
| | Primer | |
| 66 | PAH TaqMan | GTCCAGCTCTTGAAT |
| | Reverse | GGTTCTT |
| | Primer | |
| 67 | Actin FAM | AGCGGGAAATCGTGC |
| | Probe | GTGAC |
| 68 | Actin Forward | GGACCTGACTGACTA |
| | Primer | CCTCAT |
| 69 | Actin Reverse | CGTAGCACAGCTTCT |
| | Primer | CCTTAAT |
| 70 | Codon- | ATGTCTACCGCCGTG |
| | optimized | CTGGAAAATCCTGGC |
| | PAH (OPT3) | CTGGGCAGAAAGCTG |
| | | AGCGACTTCGGCCAA |
| | | GAGACAAGCTACATC |
| | | GAGGACAACTGCAAC |
| | | CAGAACGGCGCCATC |
| | | AGCCTGATCTTCAGC |
| | | CTGAAAGAAGAAGTG |
| | | GGCGCCCTGGCCAAG |
| | | GTGCTGAGACTGTTC |
| | | GAAGAGAACGACGTG |
| | | AACCTGACACACATC |
| | | GAGAGCAGACCCAGC |
| | | AGACTGAAGAAGGAC |
| | | GAGTACGAGTTCTTC |
| | | ACCCACCTGGACAAG |
| | | CGGAGCCTGCCTGCT |
| | | CTGACCAACATCATC |
| | | AAGATCCTGCGGCAC |
| | | GACATCGGCGCCACA |
| | | GTGCACGAACTGAGC |
| | | CGGGACAAGAAAAAG |
| | | GACACCGTGCCATGG |
| | | TTCCCCAGAACCATC |
| | | CAAGAGCTGGACAGA |
| | | TTCGCCAACCAGATC |
| | | CTGAGCTATGGCGCC |
| | | GAGCTGGACGCTGAT |
| | | CACCCTGGCTTTAAG |
| | | GACCCCGTGTACCGG |
| | | GCCAGAAGAAAGCAG |
| | | TTTGCCGATATCGCC |
| | | TACAACTACCGG |
| | | CACGGCCAGCCTATT |
| | | CCTCGGGTCGAGTAC |
| | | ATGGAAGAGGAAAAG |
| | | AAAACCTGGGGCACC |
| | | GTGTTCAAGACCCTG |
| | | AAGTCCCTGTACAAG |
| | | ACCCACGCCTGCTAC |
| | | GAGTACAACCACATC |
| | | TTCCCACTGCTCGAG |
| | | AAGTACTGCGGCTTC |
| | | CACGAGGACAATATC |
| | | CCTCAGCTCGAGGAC |
| | | GTGTCCCAGTTCCTG |
| | | CAGACCTGCACCGGC |
| | | TTTAGACTGAGGCCT |
| | | GTTGCCGGACTGCTG |
| | | AGCAGCAGAGATTTT |
| | | CTCGGCGGCCTGGCC |
| | | TTCAGAGTGTTCCAC |
| | | TGTACCCAGTACATC |
| | | AGACACGGCAGCAAG |
| | | CCCATGTACACCCCT |
| | | GAGCCTGATATCTGC |
| | | CACGAGCTGCTGGGA |
| | | CATGTGCCCCTGTTC |
| | | AGCGATAGAAGCTTC |
| | | GCCCAGTTCAGCCAA |
| | | GAGATCGGACTGGCT |
| | | TCTCTGGGAGCCCCT |
| | | GACGAGTACATTGAG |
| | | AAGCTGGCCACCATC |
| | | TACTGGTTCACCGTG |
| | | GAGTTCGGCCTGTGC |
| | | AAGCAGGGCGATAGC |
| | | ATCAAGGCTTATGGC |
| | | GCTGGCCTGCTGTCT |
| | | AGCTTTGGCGAGCTG |
| | | CAGTACTGTCTGAGC |
| | | GAGAAGCCTAAGCTG |
| | | CTGCCCCTGGAACTG |
| | | GAAAAGACCGCCATC |
| | | CAGAACTACACCGTG |
| | | ACCGAGTTCCAGCCT |
| | | CTGTACTACGTGGCC |
| | | GAGAGCTTCAACGAC |
| | | GCCAAAGAAAAAGTG |
| | | CGGAACTTCGCCGCC |
| | | ACCATTCCTCGGCCT |
| | | TTCAGCGTCAGATAC |
| | | GACCCCTACACACAG |
| | | CGGATCGAGGTGCTG |
| | | GACAACACACAGCAG |
| | | CTGAAAATTCTGGCC |
| | | GACAGCATCAACAGC |
| | | GAGATCGGCATCCTG |
| | | TGCAGCGCCCTGCAG |
| | | AAAATCAAGTGA |
| 71 | Codon- | ATGAGTACGGCTGTG |
| | optimized | CTCGAGAATCCAGGT |
| | PAH | TTGGGCCGAAAGCTG |
| | (OPT2/3) | TCTGATTTTGGACAG |
| | | GAGACATCTTATATT |
| | | GAAGACAACTGCAAC |
| | | CAGAATGGTGCGATA |
| | | TCCCTTATTTTTTCT |
| | | CTGAAAGAAGAAGTA |
| | | GGTGCGCTGGCAAAG |
| | | GTCTTGCGGCTGTTT |
| | | GAAGAGAACGATGTT |
| | | AATCTTACTCATATT |
| | | GAGTCCAGACCATCA |
| | | CGGCTGAAAAAAGAC |
| | | GAGTACGATCATTAA |
| | | GATCCTCCGGCATGA |
| | | CATAGGGGCGACAGT |
| | | GCATGAGCTTTCAAG |
| | | GGATAAAAAGAAAGA |
| | | TACCGTCCCCTGGTT |
| | | TCCAAGGACCATACA |
| | | AGAACTCGACCGATT |
| | | CGCGAACCAGATCCT |
| | | TTCATATGGTGCTGA |
| | | GTTGGATGCTGACCA |
| | | CCCCGGCTTCAAAGA |
| | | CCCGGTCTACCGAGC |
| | | GCGGCGGAAACAATT |
| | | TGCTGACATCGCATA |
| | | CAATTACAGGCATGG |
| | | CCAGCCAATTCCTAG |
| | | AGTAGAATACATGGA |
| | | AGAAGAGAAAAAAAC |
| | | CTGGGGTACCGTCTT |
| | | CAAGACGCTGAAATC |
| | | ATTGTATAAAACTCA |
| | | TGCATGTTACGAATA |
| | | TAACCATATTTTTCC |
| | | GTTGCTCGAGAAATA |
| | | TTGCGGGTTCCACGA |
| | | AGATAACATCCCACA |
| | | ACTCGAGGATGTATC |
| | | TCAGTTCCTCCAGAC |
| | | CTGTACGGGGTTTCG |
| | | ACTTAGGCCTGTTGC |
| | | CGGACTGCTGAGCAG |
| | | CAGAGATTTTCTCGG |
| | | CGGCCTGGCCTTCAG |
| | | AGTGTTCCACTGTAC |
| | | CCAGTACATCAGACA |
| | | CGGCAGCAAGCCCAT |
| | | GTACACCCCTGAGCC |
| | | TGATATCTGCCACGA |
| | | GCTGCTGGGACATGT |
| | | GCCCCTGTTCAGCGA |
| | | TAGAAGCTTCGCCCA |
| | | GTTCAGCCAAGAGAT |
| | | CGGACTGGCTTCTCT |
| | | GGGAGCCCCTGACGA |
| | | GTACATTGAGAAGCT |
| | | GGCCACCATCTACTG |
| | | GTTCACCGTGGAGTT |
| | | CGGCCTGTGCAAGCA |
| | | GGGCGATAGCATCAA |
| | | GGCTTATGGCGCTGG |
| | | CCTGCTGTCTAGCTT |
| | | TGGCGAGCTGCAGTA |
| | | CTGTCTGAGCGAGAA |
| | | GCCTAAGCTGCTGCC |
| | | CCTGGAACTGGAAAA |
| | | GACCGCCATCCAGAA |
| | | CTACACCGTGACCGA |
| | | GTTCCAGCCTCTGTA |
| | | CTACGTGGCCGAGAG |
| | | CTTCAACGACGCCAA |
| | | AGAAAAAGTGCGGAA |
| | | CTTCGCCGCCACCAT |
| | | TCCTCGGCCTTTCAG |
| | | CGTCAGATACGACCC |
| | | CTACACACAGCGGAT |
| | | CGAGGTGCTGGACAA |
| | | CACACAGCAGCTGAA |
| | | AATTCTGGCCGACAG |
| | | CATCAACAGCGAGAT |
| | | CGGCATCCTGTGCAG |
| | | CGCCCTGCAGAAAAT |
| | | CAAGTGA |
| 72 | Codon- | ATGTCTACCGCCGTG |
| | optimized | CTGGAAAATCCTGGC |
| | PAH | CTGGGCAGAAAGCTG |
| | (OPT3/2) | AGCGACTTCGGCCAA |
| | | GAGACAAGCTACATC |
| | | GAGGACAACTGCAAC |
| | | CAGAACGGCGCCATC |
| | | AGCCTGATCTTCAGC |
| | | CTGAAAGAAGAAGTG |
| | | GGCGCCCTGGCCAAG |
| | | GTGCTGAGACTGTTC |
| | | GAAGAGAACGACGTG |
| | | AACC |
| | | TGACACACATCGAGA |
| | | GCAGACCCAGCAGAC |
| | | TGAAGAAGGACGAGT |
| | | ACGAGTTCTTCACCC |
| | | ACCTGGACAAGCGGA |
| | | GCCTGCCTGCTCTGA |
| | | CCAACATCATCAAGA |
| | | TCCTGCGGCACGACA |
| | | TCGGCGCCACAGTGC |
| | | ACGAACTGAGCCGGG |
| | | ACAAGAAAAAGGACA |
| | | CCGTGCCATGGTTCC |
| | | CCAGAACCATCCAAG |
| | | AGCTGGACAGATTCG |
| | | CCAACCAGATCCTGA |
| | | GCTATGGCGCCGAGC |
| | | TGGACGCTGATCACC |
| | | CTGGCTTTAAGGACC |
| | | CCGTGTACCGGGCCA |
| | | GAAGAAAGCAGTTTG |
| | | CCGATATCGCCTACA |
| | | ACTACCGGCACGGCC |
| | | AGCCTATTCCTCGGG |
| | | TCGAGTACATGGAAG |
| | | AGGAAAAGAAAACCT |
| | | GGGGCACCGTGTTCA |
| | | AGACCCTGAAGTCCC |
| | | TGTACAAGACCCACG |
| | | CCTGCTACGAGTACA |
| | | ACCACATCTTCCCAC |
| | | TGCTCGAGAAGTACT |
| | | GCGGCTTCCACGAGG |
| | | ACAATATCCCTCAGC |
| | | TCGAGGACGTGTCCC |
| | | AGTTCCTGCAGACCT |
| | | GCACCGGCTTTAGAC |
| | | TGAGGCCTGTCGCGG |
| | | GTTTGCTCAGTTCTC |
| | | GAGACTTCCTGGGTG |
| | | GATTGGCGTTTCGGG |
| | | TATTCCATTGCACGC |
| | | AGTATATCCGACACG |
| | | GAAGTAAGCCAATGT |
| | | ACACGCCAGAACCCG |
| | | ATATCTGTCACGAAT |
| | | TGCTTGGACACGTTC |
| | | CTCTGTTTTCTGATC |
| | | GATCATTCGCTCAGT |
| | | TTTCACAGGAAATCG |
| | | GCCTGGCATCTTTGG |
| | | GAGCGCCGGATGAAT |
| | | ATATTGAGAAGCTCG |
| | | CTACAATTTACTGGT |
| | | TCACGGTAGAATTTG |
| | | GGTTGTGCAAGCAGG |
| | | GTGATAGTATTAAAG |
| | | CATACGGTGCGGGAT |
| | | TGCTGTCCTCATTCG |
| | | GGGAGCTTCAGTATT |
| | | GCCTGTCCGAGAAAC |
| | | CCAAGCTGTTGCCGT |
| | | TGGAATTGGAAAAAA |
| | | CCGCTATCCAAAATT |
| | | ACACAGTAACGGAGT |
| | | TCCAACCTTTGTACT |
| | | ACGTAGCCGAGTCAT |
| | | TTAACGATGCAAAGG |
| | | AGAAGGTCAGAAATT |
| | | TTGCTGCGACGATAC |
| | | CCAGACCGTTCTCAG |
| | | TAAGGTACGATCCTT |
| | | ACACTCAGAGGATTG |
| | | AAGTCCTGGATAATA |
| | | CGCAACAGCTCAAGA |
| | | TCCTGGCAGACTCCA |
| | | TAAATTCTGAAATCG |
| | | GCATCTTGTGTTCAG |
| | | CACTGCAAAAGATAA |
| | | AATAA |
| 73 | DNA | AGAACCATCCAAGAG |
| | Fragment of | |
| | OPT3 | |
| 74 | DNA | TATTCCTCGGGTCGA |
| | Fragment of | GTAC |
| | OPT3 | |
| 75 | DNA | AGAGATCGGACTGGC |
| | Fragment of | T |
| | OPT3 | |
| 76 | DNA | TCCTCGGCCTTTCAG |
| | Fragment of | |
| | OPT3 | |
| 77 | DNA | GTTAATCATTAACGC |
| | fragment | TTGTACTTTGGTACA |
| | containing | ATCGATGCGAGAACT |
| | prothrombin | TGTGCCTCCCCGTGT |
| | enhancer, | TCCTGCTCTTTGTCC |
| | human alpha- | CTCTGTCCTACTTAG |
| | 1, anti-trypsin | ACTAATATTTGCCTT |
| | promoter, | GGGTACTGCAAACAG |
| | and | GAAATGGGGGAGGGA |
| | one | CAGGAGTAGGGCGGA |
| | HNF1/HNF4 | GGGTAGCCCGGGGAT |
| | binding | TCTGCAGTGAGAGCA |
| | site | GAGGGCCAGCTAAGT |
| | | GGTACTCTCCCAGAG |
| | | ACTGTCTGACTCACG |
| | | CCACCCCCTCCACCT |
| | | TGGACACAGGACGCT |
| | | GTGGTTTCTGAGCCA |
| | | GGTACAATGACTCCT |
| | | TTCGGTAAGTGCAGT |
| | | GGAAGCTGTACACTG |
| | | CCCAGGCAAAGCGTC |
| | | CGGGCAGCGTAGGCG |
| | | GGCGACTCAGATCCC |
| | | AGCCAGTGGACTTAG |
| | | CCCCTGTTTGCTCCT |
| | | CCGATAACTGGGGTG |
| | | ACCTTGGTTAATATT |
| | | CACCAGCAGCCTCCC |
| | | CCGTTGCCCCTCTGG |
| | | ATCCACTGCTTAAAT |
| | | ACGGACGAGGACAGG |
| | | GCCCTGTCTCCTCAG |
| | | CTTCAGGCACCACCA |
| | | CTGACCTGGGACAGT |
| | | GAAT |
| 78 | Prothrombin | GCGAGAACTTGTGCC |
| | enhancer- | TCCCCGTGTTCCTGC |
| | hAAT | TCTTTGTCCCTCTGT |
| | promoter- | CCTACTTAGACTAAT |
| | | ATTTGCCTTGGGTAC |
| | | TGCAAACAGGAAATG |
| | | GGGGAGGGACAGGAG |
| | | TAGGGCGGAGGGTAG |
| | | CCCGGGGATCTTGCT |
| | | ACCAGTGGAACAGCC |
| | | ACTAAGGATTCTGCA |
| | | GTGAGAGCAGAGGGC |
| | | CAGCTA |
| | Minute Virus | AGTGGTACTCTCCCA |
| | of Mouse | GAGACTGTCTGACTC |
| | intron | ACGCCACCCCCTCCA |
| | | CCTTGGACACAGGAC |
| | | GCTGTGGTTTCTGAG |
| | | CCAGGTACAATGACT |
| | | CCTTTCGGTAAGTGC |
| | | AGTGGAAGCTGTACA |
| | | CTGCCCAGGCAAAGC |
| | | GTCCGGGCAGCGTAG |
| | | GCGGGCGACTCAGAT |
| | | CCCAGCCAGTGGACT |
| | | TAGCCCCTGTTTGCT |
| | | CCTCCGATAACTGGG |
| | | GTGACCTTGGTTAAT |
| | | ATTCACCAGCAGCCT |
| | | CCCCCGTTGCCCCTC |
| | | TGGATCCACTGCTTA |
| | | AATACGGACGAGGAC |
| | | AGGGCCCTGTCTCCT |
| | | CAGCTTCAGGCACCA |
| | | CCACTGACCTGGGAC |
| | | AGTGAATAAGAGGTA |
| | | AGGGTTTAAGGGATG |
| | | GTTGGTTGGTGGGGT |
| | | ATTAATGTTTAATTA |
| | | CCTGGAGCACCTGCC |
| | | TGAAATCACTTTTTT |
| | | TCAGGTTGG |
| 79 | hAAT | GGGGGAGGCTGCTGG |
| | promoter- | TGAATATTAACCAAG |
| | Transthyretin | GTCACCCCAGTTATC |
| | enhancer- | GGAGGAGCAAACAGG |
| | Minute | GGCTAAGTCCACCGA |
| | Virus | TGCTCTAATCTCTCT |
| | of Mouse | AGACAAGGTTCATAT |
| | intron | TTGTATGGGTTACTT |
| | | ATTCTCTCTTTGTTG |
| | | ACTAAGTCAATAATC |
| | | AGAATCAGCAGGTTT |
| | | GCAGTCAGATTGGCA |
| | | GGGATAAGCAGCCTA |
| | | GCTCAGGAGAAGTGA |
| | | GTATAAAAGCCCCAG |
| | | GCTGGGAGCAGCCAT |
| | | CAAAGAGGTAAGGGT |
| | | TTAAGGGATGGTTGG |
| | | TTGGTGGGGTATTAA |
| | | TGTTTAATTACCTGG |
| | | AGCACCTGCCTGAAA |
| | | TCACTTTTTTTCAGG |
| | | TTGG |
| 80 | Minute | AAGAGGTAAGGGTTT |
| | virus | AAGGGATGGTTGGTT |
| | of Mouse | GGTGGGGTATTAATG |
| | intron | TTTAATTACCTGGAG |
| | | CACCTGCCTGAAATC |
| | | ACTTTTTTTCAGGTT |
| | | GG |
| 81 | Transthyretin | CCGATGCTCTAATCT |
| | enhancer | CTCTAGACAAGGTTC |
| | | ATATTTGTATGGGTT |
| | | ACTTATTCTCTCTTT |
| | | GTTGACTAAGTCAAT |
| | | AATCAGAATCAGCAG |
| | | GTTTGCAGTCAGATT |
| | | GGCAGGGATAAGCAG |
| | | CCTAGCTCAGGAGAA |
| | | GTGAGTATAAAAGCC |
| | | CCAGGCTGGGAGCAG |
| | | CCATCA |
| 82 | hAAT | GGGGGAGGCTGCTGG |
| | promoter | TGAATATTAACCAAG |
| | | GTCACCCCAGTTATC |
| | | GGAGGAGCAAACAGG |
| | | GGCTAAGTCCA |
| 83 | PAH | ATGTCTACCGCCGTG |
| | optimized | CTGGAAAATCCTGGC |
| | version | CTGGGCAGAAAGCTG |
| | 3-PAH | AGCGACTTCGGCCAA |
| | 3′UTR | GAGACAAGCTACATC |
| | | GAGGACAACTGCAAC |
| | | CAGAACGGCGCCATC |
| | | AGCCTGATCTTCAGC |
| | | CTGAAAGAAGAAGTG |
| | | GGCGCCCTGGCCAAG |
| | | GTGCTGAGACTGTTC |
| | | GAAGAGAACGACGTG |
| | | AACCTGACACACATC |
| | | GAGAGCAGACCCAGC |
| | | AGACTGAAGAAGGAC |
| | | GAGTACGAGTTCTTC |
| | | ACCCACCTGGACAAG |
| | | CGGAGCCTGCCTGCT |
| | | CTGACCAACATCATC |
| | | AAGATCCTGCGGCAC |
| | | GACATCGGCGCCACA |
| | | GTGCACGAACTGAGC |
| | | CGGGACAAGAAAAAG |
| | | GACACCGTGCCATGG |
| | | TTCCCCAGAACCATC |
| | | CAAGAGCTGGACAGA |
| | | TTCGCCAACCAGATC |
| | | CTGAGCTATGGCGCC |
| | | GAGCTGGACGCTGAT |
| | | CACCCTGGCTTTAAG |
| | | GACCCCGTGTACCGG |
| | | GCCAGAAGAAAGCAG |
| | | TTTGCCGATATCGCC |
| | | TACAACTACCGGCAC |
| | | GGCCAGCCTATTCCT |
| | | CGGGTCGAGTACATG |
| | | GAAGAGGAAAAGAAA |
| | | ACCTGGGGCACCGTG |
| | | TTCAAGACCCTGAAG |
| | | TCCCTGTACAAGACC |
| | | CACGCCTGCTACGAG |
| | | TACAACCACATCTTC |
| | | CCACTGCTCGAGAAG |
| | | TACTGCGGCTTCCAC |
| | | GAGGACAATATCCCT |
| | | CAGCTCGAGGACGTG |
| | | TCCCAGTTCCTGCAG |
| | | ACCTGCACCGGCTTT |
| | | AGACTGAGGCCTGTT |
| | | GCCGGACTGCTGAGC |
| | | AGCAGAGATTTTCTC |
| | | GGCGGCCTGGCCTTC |
| | | AGAGTGTTCCACTGT |
| | | ACCCAGTACATCAGA |
| | | CACGGCAGCAAGCCC |
| | | ATGTACACCCCTGAG |
| | | CCTGATATCTGCCAC |
| | | GAGCTGCTGGGACAT |
| | | GTGCCCCTGTTCAGC |
| | | GATAGAAGCTTCGCC |
| | | CAGTTCAGCCAAGAG |
| | | ATCGGACTGGCTTCT |
| | | CTGGGAGCCCCTGAC |
| | | GAGTACATTGAGAAG |
| | | CTGGCCACCATCTAC |
| | | TGGTTCACCGTGGAG |
| | | TTCGGCCTGTGCAAG |
| | | CAGGGCGATAGCATC |
| | | AAGGCTTATGGCGCT |
| | | GGCCTGCTGTCTAGC |
| | | TTTGGCGAGCTGCAG |
| | | TACTGTCTGAGCGAG |
| | | AAGCCTAAGCTGCTG |
| | | CCCCTGGAACTGGAA |
| | | AAGACCGCCATCCAG |
| | | AACTACACCGTGACC |
| | | GAGTTCCAGCCTCTG |
| | | TACTACGTGGCCGAG |
| | | AGCTTCAACGACGCC |
| | | AAAGAAAAAGTGCGG |
| | | AACTT |
| | | CGCCGCCACCATTCC |
| | | TCGGCCTTTCAGCGT |
| | | CAGATACGACCCCTA |
| | | CACACAGCGGATCGA |
| | | GGTGCTGGACAACAC |
| | | ACAGCAGCTGAAAAT |
| | | TCTGGCCGACAGCAT |
| | | CAACAGCGAGATCGG |
| | | CATCCTGTGCAGCGC |
| | | CCTGCAGAAAATCAA |
| | | GTGAGTCGACAGCCA |
| | | TGGACAGAATGTGGT |
| | | CTGTCAGCTGTGAAT |
| | | CTGTTGATGGAGATC |
| | | CAACTATTTCTTTCA |
| | | TCAGAAAAAGTCCGA |
| | | AAAGCAAACCTTAAT |
| | | TTGAAATAACAGCCT |
| | | TAAATCCTTTACAAG |
| | | ATGGAGAAACAACAA |
| | | ATAAGTCAAAATAAT |
| | | CTGAAATGACAGGAT |
| | | ATGAGTACATACTCA |
| | | AGAGCATAATGGTAA |
| | | ATCTTTTGGGGTCAT |
| | | CTTTGATTTAGAGAT |
| | | GATAATCCCATACTC |
| | | TCAATTGAGTTAAAT |
| | | CAGTAATCTGTCGCA |
| | | TTTCATCAAGATTA |
| 84 | PAH | ATGTCTACCGCCGTG |
| | optimized | CTGGAAAATCCTGGC |
| | version 3- | CTGGGCAGAAAGCTG |
| | Albumin | AGCGACTTCGGCCAA |
| | 3′UTR | GAGACAAGCTACATC |
| | | GAGGACAACTGCAAC |
| | | CAGAACGGCGCCATC |
| | | AGCCTGATCTTCAGC |
| | | CTGAAAGAAGAAGTG |
| | | GGCGCCCTGGCCAAG |
| | | GTGCTGAGACTGTTC |
| | | GAAGAGAACGACGTG |
| | | AACCTGACACACATC |
| | | GAGAGCAGACCCAGC |
| | | AGACTGAAGAAGGAC |
| | | GAGTACGAGTTCTTC |
| | | ACCCACCTGGACAAG |
| | | CGGAGCCTGCCTGCT |
| | | CTGACCAACATCATC |
| | | AAGATCCTGCGGCAC |
| | | GACATCGGCGCCACA |
| | | GTGCACGAACTGAGC |
| | | CGGGACAAGAAAAAG |
| | | GACACCGTGCCATGG |
| | | TTCCCCAGAACCATC |
| | | CAAGAGCTGGACAGA |
| | | TTCGCCAACCAGATC |
| | | CTGAGCTATGGCGCC |
| | | GAGCTGGACGCTGAT |
| | | CACCCTGGCTTTAAG |
| | | GACCCCGTGTACCGG |
| | | GCCAGAAGAAAGCAG |
| | | TTTGCCGATATCGCC |
| | | TACAACTACCGGCAC |
| | | GGCCAGCCTATTCCT |
| | | CGGGTCGAGTACATG |
| | | GAAGAGGAAAAGAAA |
| | | ACCTGGGGCACCGTG |
| | | TTCAAGACCCTGAAG |
| | | TCCCTGTACAAGACC |
| | | CACGCCTGCTACGAG |
| | | TACAACCACATCTTC |
| | | CCACTGCTCGAGAAG |
| | | TACTGCGGCTTCCAC |
| | | GAGGACAATATCCCT |
| | | CAGCTCGAGGACGTG |
| | | TCCCAGTTCCTGCAG |
| | | ACCTGCACCGGCTTT |
| | | AGACTGAGGCCTGTT |
| | | GCCGGACTGCTGAGC |
| | | AGCAGAGATTTTCTC |
| | | GGCGGCCTGGCCTTC |
| | | AGAGTGTTCCACTGT |
| | | ACCCAGTACATCAGA |
| | | CACGGCAGCAAGCCC |
| | | ATGTACACCCCTGAG |
| | | CCTGATATCTGCCAC |
| | | GAGCTGCTGGGACAT |
| | | GTGCCCCTGTTCAGC |
| | | GATAGAAGCTTCGCC |
| | | CAGTTCAGCCAAGAG |
| | | ATCGGACTGGCTTCT |
| | | CTGGGAGCCCCTGAC |
| | | GAGTACATTGAGAAG |
| | | CTGGCCACCATCTAC |
| | | TGGTTCACCGTGGAG |
| | | TTCGGCCTGTGCAAG |
| | | CAGGGCGATAGCATC |
| | | AAGGCTTATGGCGCT |
| | | GGCCTGCTGTCTAGC |
| | | TTTGGCGAGCTGCAG |
| | | TACTGTCTGAGCGAG |
| | | AAGCCTAAGCTGCTG |
| | | CCCCTGGAACTGGAA |
| | | AAGACCGCCATCCAG |
| | | AACTACACCGTGACC |
| | | GAGTTCCAGCCTCTG |
| | | TACTACGTGGCCGAG |
| | | AGCTTCAACGACGCC |
| | | AAAGAAAAAGTGCGG |
| | | AACTTCGCCGCCACC |
| | | ATTCCTCGGCCTTTC |
| | | AGCGTCAGATACGAC |
| | | CCCTACACACAGCGG |
| | | ATCGAGGTGCTGGAC |
| | | AACACACAGCAGCTG |
| | | AAAATTCTGGCCGAC |
| | | AGCATCAACAGCGAG |
| | | ATCGGCATCCTGTGC |
| | | AGCGCCCTGCAGAAA |
| | | ATCAAGTGAGTCGAC |
| | | ATTCAGCAGCCGTAA |
| | | GTCTAGGACAGGCTT |
| | | AAATTGTTTTCACTG |
| | | GTGTAAATTGCAGAA |
| | | AGATGATCTAAGTAA |
| | | TTTGGCATTTATTTT |
| | | AATAGGTTTGAAAAA |
| | | CACATGCCATTTTAC |
| | | AAATAAGACTTATAT |
| | | TTGTCCTTTTGTTTT |
| | | TCAGCCTACCATGAG |
| | | AATAAGAGAAAGAAA |
| | | ATGAAGATCAAAAGC |
| | | TTATTCATCTGTTTT |
| | | TCTTTTTCGTTGGTG |
| | | TAAAGCCAACACCCT |
| | | GTCTAAAAAACATAA |
| | | ATTTCTTTAATCATT |
| | | TTGCCTCTTTTCTCT |
| | | GTGCTTCAATTAATA |
| | | AAAAATGGAAAGAAT |
| | | CTAATAGAGTGGTAC |
| | | AGCACTGTTATTTTT |
| | | CAAAGATGTGTTGCT |
| | | ATCCTGAAAATTCTG |
| | | TAGGTTCTGTGGAAG |
| | | TTCCAGTGTTCTCTC |
| | | TTATTCCACTTCGGT |
| | | AGAGGATTTCTAGTT |
| | | TCTTGTGGGCTAATT |
| | | AAATAAATCATTAAT |
| | | ACTCTTCTAAGTTAT |
| | | GGATTATAAACATTC |
| | | AAAATAATATTTTGA |
| | | CATTATGATAATTCT |
| | | GAATAAAAGAACAAA |
| | | AACCATGGTATAGGT |
| | | AAGGAATATAAAACA |
| | | TGGCTTTTACCTTAG |
| | | AAAAAACAATTCTAA |
| | | AATTCATATGGAATC |
| | | AAAAAAGAGCCTGCA |
| 85 | PAH 3′UTR | AGCCATGGACAGAAT |
| | | GTGGTCTGTCAGCTG |
| | | TGAATCTGTTGATGG |
| | | AGATCCAACTATTTC |
| | | TTTCATCAGAAAAAG |
| | | TCCGAAAAGCAAACC |
| | | TTAATTTGAAATAAC |
| | | AGCCTTAAATCCTTT |
| | | ACAAGATGGAGAAAC |
| | | AACAAATAAGTCAAA |
| | | ATAATCTGAAATGAC |
| | | AGGATATGAGTACAT |
| | | ACTCAAGAGCATAAT |
| | | GGTAAATCTTTTGGG |
| | | GTCATCTTTGATTTA |
| | | GAGATGATAATCCCA |
| | | TACTCTCAATTGAGT |
| 86 | Albumin | ATTCAGCAGCCGTAA |
| | 3′UTR | GTCTAGGACAGGCTT |
| | | AAATTGTTTTCACTG |
| | | GTGTAAATTGCAGAA |
| | | AGATGATCTAAGTAA |
| | | TTTGGCATTTATTTT |
| | | AATAGGTTTGAAAAA |
| | | CACATGCCATTTTAC |
| | | AAATAAGACTTATAT |
| | | TTGTCCTTTTGTTTT |
| | | TCAGCCTACCATGAG |
| | | AATAAGAGAAAGAAA |
| | | ATGAAGATCAAAAGC |
| | | TTATTCATCTGTTTT |
| | | TCTTTTTCGTTGGTG |
| | | TAAAGCCAACACCCT |
| | | GTCTAAAAAACATAA |
| | | ATTTCTTTAATCATT |
| | | TTGCCTCTTTTCTCT |
| | | GTGCTTCAATTAATA |
| | | AAAAATGGAAAGAAT |
| | | CTAATAGAGTGGTAC |
| | | AGCACTGTTATTTTT |
| | | CAAAGATGTGTTGCT |
| | | ATCCTGAAAATTCTG |
| | | TAGGTTCTGTGGAAG |
| | | TTCCAGTGTTCTCTC |
| | | TTATTCCACTTCGGT |
| | | AGAGGATTTCTAGTT |
| | | TCTTGTGGGCTAATT |
| | | AAATAAATCATTAAT |
| | | ACTCTTCTAAGTTAT |
| | | GGATTATAAACATTC |
| | | AAAATAATATTTTGA |
| | | CATTATGATAATTCT |
| | | GAATAAAAGAACAAA |
| | | AACCATGGTATAGGT |
| | | AAGGAATATAAAACA |
| | | TGGCTTTTACCTTAG |
| | | AAAAAACAATTCTAA |
| | | AATTCATATGGAATC |
| | | AAAAAAGAGCCTGCA |
| 87 | WPREs | AATCAACCTCTGGAT |
| | (WPRE | TACAAAATTTGTGAA |
| | without X- | AGATTGACTGATATT |
| | protein | CTTAACTATGTTGCT |
| | sequence) | CCTTTTACGCTGTGT |
| | | GGATATGCTGCTTTA |
| | | ATGCCTCTGTATCAT |
| | | GCTATTGCTTCCCGT |
| | | ACGGCTTTCGTTTTC |
| | | TCCTCCTTGTATAAA |
| | | TCCTGGTTGCTGTCT |
| | | CTTTATGAGGAGTTG |
| | | TGGCCCGTTGTCCGT |
| | | CAACGTGGCGTGGTG |
| | | TGCTCTGTGTTTGCT |
| | | GACGCAACCCCCACT |
| | | GGCTGGGGCATTGCC |
| | | ACCACCTGTCAACTC |
| | | CTTTCTGGGACTTTC |
| | | GCTTTCCCCCTCCCG |
| | | ATCGCCACGGCAGAA |
| | | CTCATCGCCGCCTGC |
| | | CTTGCCCGCTGCTGG |
| | | ACAGGGGCTAGGTTG |
| | | CTGGGCACTGATAAT |
| | | TCCGTGGTGTTGTCG |
| | | GTACC |

What is claimed is:

1. A viral vector comprising a therapeutic cargo portion, wherein the therapeutic cargo portion comprises:
a codon-optimized PAH sequence or variant thereof;
a promoter; and
a liver-specific enhancer,
wherein the codon-optimized PAH sequence or variant thereof is operatively controlled by both the promoter and the liver-specific enhancer.

2. The viral vector of claim 1, wherein the codon-optimized PAH sequence or variant thereof comprises a sequence having at least 80 percent, at least 85 percent, at least 90 percent, or at least 95 percent sequence identity with SEQ ID NO: 70.

3. The viral vector of claim 2, wherein the codon-optimized PAH sequence or variant thereof comprises the sequence of SEQ ID NO: 70.

4. The viral vector of claim 1, wherein the codon-optimized PAH sequence or variant thereof comprises a sequence having at least 80 percent, at least 85 percent, at least 90 percent, or at least 95 percent sequence identity with SEQ ID NO: 71.

5. The viral vector of claim 4, wherein the codon-optimized PAH sequence or variant thereof comprises the sequence of SEQ ID NO: 71.

6. The viral vector of claim 1, wherein the codon-optimized PAH sequence or variant thereof comprises a sequence having at least 80 percent, at least 85 percent, at least 90 percent, or at least 95 percent sequence identity with SEQ ID NO: 72.

7. The viral vector of claim 6, wherein the codon-optimized PAH sequence or variant thereof comprises the sequence of SEQ ID NO: 72.

8. The viral vector of claim 1, wherein the codon-optimized PAH sequence or variant thereof comprises a sequence having at least 80 percent, at least 85 percent, at least 90 percent, or at least 95 percent sequence identity with SEQ ID NO: 73.

9. The viral vector of claim 8, wherein the codon-optimized PAH sequence or variant thereof comprises the sequence of SEQ ID NO: 73.

10. The viral vector of claim 1, wherein the codon-optimized PAH sequence or variant thereof comprises a sequence having at least 80 percent, at least 85 percent, at least 90 percent, or at least 95 percent sequence identity with SEQ ID NO: 74.

11. The viral vector of claim 10, wherein the codon-optimized PAH sequence or variant thereof comprises the sequence of SEQ ID NO: 74.

12. The viral vector of claim 1, wherein the a codon-optimized PAH sequence or variant thereof comprises a sequence having at least 80 percent, at least 85 percent, at least 90 percent, or at least 95 percent sequence identity with SEQ ID NO: 75.

13. The viral vector of claim 12, wherein the codon-optimized PAH sequence or variant thereof comprises the sequence of SEQ ID NO: 75.

14. The viral vector of claim 1, wherein the codon-optimized PAH sequence or variant thereof comprises a sequence having at least 80 percent, at least 85 percent, at least 90 percent, or at least 95 percent sequence identity with SEQ ID NO: 76.

15. The viral vector of claim 14, wherein the codon-optimized PAH sequence or variant thereof comprises the sequence of SEQ ID NO: 76.

16. The viral vector of claim 1, wherein the liver-specific enhancer comprises a prothrombin enhancer.

17. The viral vector of claim 1, wherein the promoter comprises a liver-specific promoter.

18. The viral vector of claim 17, wherein the liver-specific promoter comprises a hAAT promoter.

19. The viral vector of claim 1, wherein the therapeutic cargo portion further comprises a beta globin intron.

20. The viral vector of claim 1, wherein the therapeutic cargo portion further comprises at least one small RNA sequence.

21. The viral vector of claim 1, wherein the viral vector is a lentiviral vector or an adeno-associated viral vector.

22. The viral vector of claim 21, wherein the viral vector a lentiviral vector.

23. A lentiviral particle produced by a packaging cell and capable of infecting a target cell, the lentiviral particle comprising an envelope protein capable of infecting a target cell; and the viral vector of claim 1.

24. A method of treating phenylketonuria (PKU) in a subject, the method comprising administering to the subject a therapeutically effective amount of the lentiviral particle of claim 23.

25. Use of a codon-optimized PAH sequence or variant thereof for treating PKU in a subject.


SEQUENCE LISTING (87 sequences)

SEQ ID NO: 1 - DNA, 1359 bp, Artificial Sequence
  hPAH
atgtccactgcggtcctggaaaacccaggcttgggcaggaaactctctgactttggacaggaaacaagctatattgaagacaactgcaatcaaaatggtgccatatcactgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcaattcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctgtattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaagtaa

SEQ ID NO: 2 - DNA, 1359 bp, Artificial Sequence
  Codon-optimized PAH (Opt2)
atgagtacggctgtgctcgagaatccaggtttgggccgaaagctgtctgattttggacaggagacatcttatattgaagacaactgcaaccagaatggtgcgatatcccttattttttctctgaaagaagaagtaggtgcgctggcaaaggtcttgcggctgtttgaagagaacgatgttaatcttactcatattgagtccagaccatcacggctgaaaaaagacgagtacgaattttttactcacttggacaaacgaagcttgccggctcttactaatatcattaagatcctccggcatgacataggggcgacagtgcatgagctttcaagggataaaaagaaagataccgtcccctggtttccaaggaccatacaagaactcgaccgattcgcgaaccagatcctttcatatggtgctgagttggatgctgaccaccccggcttcaaagacccggtctaccgagcgcggcggaaacaatttgctgacatcgcatacaattacaggcatggccagccaattcctagagtagaatacatggaagaagagaaaaaaacctggggtaccgtcttcaagacgctgaaatcattgtataaaactcatgcatgttacgaatataaccatatttttccgttgctcgagaaatattgcgggttccacgaagataacatcccacaactcgaggatgtatctcagttcctccagacctgtacggggtttcgacttaggcctgtcgcgggtttgctcagttctcgagacttcctgggtggattggcgtttcgggtattccattgcacgcagtatatccgacacggaagtaagccaatgtacacgccagaacccgatatctgtcacgaattgcttggacacgttcctctgttttctgatcgatcattcgctcagttttcacaggaaatcggcctggcatctttgggagcgccggatgaatatattgagaagctcgctacaatttactggttcacggtagaatttgggttgtgcaagcagggtgatagtattaaagcatacggtgcgggattgctgtcctcattcggggagcttcagtattgcctgtccgagaaacccaagctgttgccgttggaattggaaaaaaccgctatccaaaattacacagtaacggagttccaacctttgtactacgtagccgagtcatttaacgatgcaaaggagaaggtcagaaattttgctgcgacgatacccagaccgttctcagtaaggtacgatccttacactcagaggattgaagtcctggataatacgcaacagctcaagatcctggcagactccataaattctgaaatcggcatcttgtgttcagcactgcaaaagataaaataa

SEQ ID NO: 3 - DNA, 120 bp, Artificial Sequence
  Prothrombin enhancer (Pro)
gcgagaacttgtgcctccccgtgttcctgctctttgtccctctgtcctacttagactaatatttgccttgggtactgcaaacaggaaatgggggagggacaggagtagggcggagggtag

SEQ ID NO: 4 - DNA, 397 bp, Artificial Sequence
  Human alpha-1 anti-trypsin promoter (hAAT)
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 5 - DNA, 573 bp, Artificial Sequence
  Rabbit beta globin intron
gtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacag

SEQ ID NO: 6 - DNA, 544 bp, Artificial Sequence
  Human beta globin intron
ggatcctgagaacttcagggtgagtctatgggacgcttgatgttttctttccccttcttttctatggttaagttcatgtcataggaaggggataagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaag

SEQ ID NO: 7 - DNA, 13 bp, Artificial Sequence
  1X Hepatocyte Nuclear Factor 1 (1XHNF1)
gttaatcattaac

SEQ ID NO: 8 - DNA, 65 bp, Artificial Sequence
  5X Hepatocyte Nuclear Factor 1 (5XHNF1)
gttaatcattaacgttaatcattaacgttaatcattaacgttaatcattaacgttaatcattaac

SEQ ID NO: 9 - DNA, 30 bp, Artificial Sequence
  1X Hepatocyte Nuclear Factor 1/4 (1XHNF1/4)
gttaatcattaacgcttgtactttggtaca

SEQ ID NO: 10 - DNA, 90 bp, Artificial Sequence
  3X Hepatocyte Nuclear Factor 1/4 (3XHNF1/4)
gttaatcattaacgcttgtactttggtacagttaatcattaacgcttgtactttggtacagttaatcattaacgcttgtactttggtaca

SEQ ID NO: 11 - DNA, 53 bp, Artificial Sequence
  PAH shRNA sequence #1
tcgcatttcatcaagattaatctcgagattaatcttgatgaaatgcgattttt

SEQ ID NO: 12 - DNA, 53 bp, Artificial Sequence
  PAH shRNA sequence #2
actcataaaggagcatataagctcgagcttatatgctcctttatgagtttttt

SEQ ID NO: 13 - DNA, 228 bp, Artificial Sequence
  Rous Sarcoma virus (RSV) promoter
gtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacg

SEQ ID NO: 14 - DNA, 180 bp, Artificial Sequence
  5 Long terminal repeat (LTR)
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 15 - DNA, 41 bp, Artificial Sequence
  Psi Packaging signal (RNA packaging site)
tacgccaaaaattttgactagcggaggctagaaggagagag

SEQ ID NO: 16 - DNA, 233 bp, Artificial Sequence
  Rev response element (RRE)
aggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcc

SEQ ID NO: 17 - DNA, 118 bp, Artificial Sequence
  Central polypurine tract (cPPT) (polypurine tract)
ttttaaaagaaaaggggggattggggggtacagtgcaggggaaagaatagtagacataatagcaacagacatacaaactaaagaattacaaaaacaaattacaaaattcaaaatttta

SEQ ID NO: 18 - DNA, 592 bp, Artificial Sequence
  Long WPRE sequence
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 19 - DNA, 250 bp, Artificial Sequence
  delta U3 3LTR
tggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtca

SEQ ID NO: 20 - DNA, 217 bp, Artificial Sequence
  H1 Promoter
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactt

SEQ ID NO: 21 - DNA, 642 bp, Artificial Sequence
  CMV enhancer/chicken beta actin promoter
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 22 - DNA, 1503 bp, Artificial Sequence
  HIV Gag
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataa

SEQ ID NO: 23 - DNA, 1872 bp, Artificial Sequence
  HIV Pol
atgaatttgccaggaagatggaaaccaaaaatgatagggggaattggaggttttatcaaagtaggacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaaatatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagatgggttggtcagtgctggaatcaggaaagtacta

SEQ ID NO: 24 - DNA, 867 bp, Artificial Sequence
  HIV Integrase (HIV Int)
tttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaa

SEQ ID NO: 25 - DNA, 234 bp, Artificial Sequence
  HIV RRE
aggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcct

SEQ ID NO: 26 - DNA, 351 bp, Artificial Sequence
  HIV Rev
atggcaggaagaagcggagacagcgacgaagaactcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatag

SEQ ID NO: 27 - DNA, 615 bp, Artificial Sequence
  CMV Promoter
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactg

SEQ ID NO: 28 - DNA, 1536 bp, Artificial Sequence
  Vesicular stomatitis Indiana virus glycoprotein VSV-G
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtga

SEQ ID NO: 29 - DNA, 141 bp, Artificial Sequence
  Left ITR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 30 - DNA, 228 bp, Artificial Sequence
  PolyA Element
gactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggc

SEQ ID NO: 31 - DNA, 141 bp, Artificial Sequence
  Right ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 32 - DNA, 1470 bp, Artificial Sequence
  E2A Element
ttaaaagtcgaaggggttctcgcgctcgtcgttgtgcgccgcgctggggagggccacgttgcggaactggtacttgggctgccacttgaactcggggatcaccagtttgggcactggggtctcggggaaggtctcgctccacatgcgccggctcatctgcagggcgcccagcatgtcaggcgcggagatcttgaaatcgcagttggggccggtgctctgcgcgcgcgagttgcggtacactgggttgcagcactggaacaccatcagactggggtacttcacactagccagcacgctcttgtcgctgatctgatccttgtccaggtcctcggcgttgctcaggccgaacggggtcatcttgcacagctggcggcccaggaagggcacgctctgaggcttgtggttacactcgcagtgcacgggcatcagcatcatccccgcgccgcgctgcatattcgggtagagggccttgacgaaggccgcgatctgcttgaaagcttgctgggccttggccccctcgctgaaaaacaggccgcagctcttcccgctgaactgattattcccgcacccggcatcatggacgcagcagcgcgcgtcatggctggtcagttgcaccacgctccgtccccagcggttctgggtcaccttggccttgctgggttgctccttcagcgcacgctgcccgttctcactggtcacatccatctccaccacgtggtccttgtggatcatcaccgtcccatgcagacacttgagctggccttccacctcggtgcagccgtggtcccacagggcactgccggtgcactcccagttcttgtgcgcgatcccgctgtggctgaagatgtaaccttgcaacaggcgacccatgatggtgctaaagctcttctgggtggtgaaggtcagttgcagaccgcgggcctcctcgttcatccaggtctggcacatcttttggaagatctcggtctgctcgggcatgagcttgtaagcatcgcgcaggccgctgtcgacgcggtagcgttccatcagcacattcatggtatccatgcccttctcccaggacgagaccagaggcagactcagggggttgcgcacgttcaggacaccgggggtcgcgggctcgacgatgcgttttccgtccttgccttccttcaacagaaccggcggctggctgaatcccactcccacgatcacggcttcttcctggggcatctcttcgtctgggtctaccttggtcacatgcttggtctttctggcttgcttcttttttggagggctgtccacggggaccacgtcctcctcggaagacccggatcccacccgctgatactttcggcgcttggttggcagaggaggtggcggcgaggggctcctctcctgctccggcggatagcgcgctgaaccgtggccccggggcggagtggcctctcggtccatgaaccggcgcacgtcctgactgccgccggccat

SEQ ID NO: 33 - DNA, 393 bp, Artificial Sequence
  E4 element
tcatgtatctttattgatttttacaccagcacgggtagtcagtctcccaccaccagcccatttcacagtgtaaacaattctctcagcacgggtggccttaaatagggcaatattctgattagtgcgggaactggacttggggtctataatccacacagtttcctggcgagccaaacgggggtcggtgattgagatgaagccgtcctctgaaaagtcatccaagcgagcctcacagtccaaggtcacagtattatgataatctgcatgatcacaatcgggcaacaggggatgttgttcagtcagtgaagccctggtttcctcatcagatcgtggtaaacgggccctgcgatatggatgatggcggagcgagctggattgaatctcggtttgcat

SEQ ID NO: 34 - DNA, 162 bp, Artificial Sequence
  VA RNA
agcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttt

SEQ ID NO: 35 - DNA, 2208 bp, Artificial Sequence
  AAV2 Rep
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 36 - DNA, 1866 bp, Artificial Sequence
  AAV2 Cap
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 37 - DNA, 1608 bp, Artificial Sequence
  AAV8 Cap
atggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 38 - DNA, 2214 bp, Artificial Sequence
  AAV DJ Cap
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctccgaccaccttcaaccagtcaaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 39 - DNA, 960 bp, Artificial Sequence
  Chicken beta actin intron
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgg

SEQ ID NO: 40 - DNA, 448 bp, Artificial Sequence
  Rabbit beta globin poly A
agatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatc

SEQ ID NO: 41 - DNA, 31 bp, Artificial Sequence
  Forward Primer
taagcagaattcatgaatttgccaggaagat

SEQ ID NO: 42 - DNA, 36 bp, Artificial Sequence
  Reverse Primer
ccatacaatgaatggacactaggcggccgcacgaat

SEQ ID NO: 43 - DNA, 2745 bp, Artificial Sequence
  Gag, Pol, Integrase fragment
gaattcatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaa

SEQ ID NO: 44 - DNA, 1586 bp, Artificial Sequence
  DNA Fragment containing the RRE, REV, and rabbit beta globin poly A sequence
tctagaatggcaggaagaagcggagacagcgacgaagagctcatcagaacagtcagactcatcaagcttctctatcaaagcaacccacctcccaatcccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagaggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcagcggccgccccggg

SEQ ID NO: 45 - DNA, 1614 bp, Artificial Sequence
  DNA fragment containing the CAG enhancer/ promoter/intron sequence
acgcgttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgggaattc

SEQ ID NO: 46 - DNA, 884 bp, Artificial Sequence
  RSV promoter and HIV Rev
caattgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcacctccaagctcgagctcgtttagtgaaccgtcagatcgcctggagacgccatccacgctgttttgacctccatagaagacaccgggaccgatccagcctcccctcgaagctagcgattaggcatctcctatggcaggaagaagcggagacagcgacgaagaactcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtctaga

SEQ ID NO: 47 - DNA, 1104 bp, Artificial Sequence
  Elongation Factor-1 alpha (EF-1 alpha) promoter
ccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 48 - DNA, 511 bp, Artificial Sequence
  PGK Promoter
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 49 - DNA, 1162 bp, Artificial Sequence
  UbC Promoter
gcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaa

SEQ ID NO: 50 - DNA, 120 bp, Artificial Sequence
  SV40 Poly A
gtttattgcagcttataatggttacaaataaagcaatagcatcacaaatttcacaaataaagcatttttttcactgcattctagttgtggtttgtccaaactcatcaatgtatcttatca

SEQ ID NO: 51 - DNA, 228 bp, Artificial Sequence
  bHG Poly
agactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 52 - DNA, 1695 bp, Artificial Sequence
  RD114 Envelope
atgaaactcccaacaggaatggtcattttatgtagcctaataatagttcgggcagggtttgacgacccccgcaaggctatcgcattagtacaaaaacaacatggtaaaccatgcgaatgcagcggagggcaggtatccgaggccccaccgaactccatccaacaggtaacttgcccaggcaagacggcctacttaatgaccaaccaaaaatggaaatgcagagtcactccaaaaaatctcacccctagcgggggagaactccagaactgcccctgtaacactttccaggactcgatgcacagttcttgttatactgaataccggcaatgcagggcgaataataagacatactacacggccaccttgcttaaaatacggtctgggagcctcaacgaggtacagatattacaaaaccccaatcagctcctacagtccccttgtaggggctctataaatcagcccgtttgctggagtgccacagcccccatccatatctccgatggtggaggacccctcgatactaagagagtgtggacagtccaaaaaaggctagaacaaattcataaggctatgcatcctgaacttcaataccaccccttagccctgcccaaagtcagagatgaccttagccttgatgcacggacttttgatatcctgaataccacttttaggttactccagatgtccaattttagccttgcccaagattgttggctctgtttaaaactaggtacccctacccctcttgcgatacccactccctctttaacctactccctagcagactccctagcgaatgcctcctgtcagattatacctcccctcttggttcaaccgatgcagttctccaactcgtcctgtttatcttcccctttcattaacgatacggaacaaatagacttaggtgcagtcacctttactaactgcacctctgtagccaatgtcagtagtcctttatgtgccctaaacgggtcagtcttcctctgtggaaataacatggcatacacctatttaccccaaaactggacaggactttgcgtccaagcctccctcctccccgacattgacatcatcccgggggatgagccagtccccattcctgccattgatcattatatacatagacctaaacgagctgtacagttcatccctttactagctggactgggaatcaccgcagcattcaccaccggagctacaggcctaggtgtctccgtcacccagtatacaaaattatcccatcagttaatatctgatgtccaagtcttatccggtaccatacaagatttacaagaccaggtagactcgttagctgaagtagttctccaaaataggaggggactggacctactaacggcagaacaaggaggaatttgtttagccttacaagaaaaatgctgtttttatgctaacaagtcaggaattgtgagaaacaaaataagaaccctacaagaagaattacaaaaacgcagggaaagcctggcatccaaccctctctggaccgggctgcagggctttcttccgtacctcctacctctcctgggacccctactcaccctcctactcatactaaccattgggccatgcgttttcaatcgattggtccaatttgttaaagacaggatctcagtggtccaggctctggttttgactcagcaatatcaccagctaaaacccatagagtacgagccatga

SEQ ID NO: 53 - DNA, 2013 bp, Artificial Sequence
  GALV Envelope
atgcttctcacctcaagcccgcaccaccttcggcaccagatgagtcctgggagctggaaaagactgatcatcctcttaagctgcgtattcggagacggcaaaacgagtctgcagaataagaacccccaccagcctgtgaccctcacctggcaggtactgtcccaaactggggacgttgtctgggacaaaaaggcagtccagcccctttggacttggtggccctctcttacacctgatgtatgtgccctggcggccggtcttgagtcctgggatatcccgggatccgatgtatcgtcctctaaaagagttagacctcctgattcagactatactgccgcttataagcaaatcacctggggagccatagggtgcagctaccctcgggctaggaccaggatggcaaattcccccttctacgtgtgtccccgagctggccgaacccattcagaagctaggaggtgtggggggctagaatccctatactgtaaagaatggagttgtgagaccacgggtaccgtttattggcaacccaagtcctcatgggacctcataactgtaaaatgggaccaaaatgtgaaatgggagcaaaaatttcaaaagtgtgaacaaaccggctggtgtaaccccctcaagatagacttcacagaaaaaggaaaactctccagagattggataacggaaaaaacctgggaattaaggttctatgtatatggacacccaggcatacagttgactatccgcttagaggtcactaacatgccggttgtggcagtgggcccagaccctgtccttgcggaacagggacctcctagcaagcccctcactctccctctctccccacggaaagcgccgcccacccctctacccccggcggctagtgagcaaacccctgcggtgcatggagaaactgttaccctaaactctccgcctcccaccagtggcgaccgactctttggccttgtgcagggggccttcctaaccttgaatgctaccaacccaggggccactaagtcttgctggctctgtttgggcatgagccccccttattatgaagggatagcctcttcaggagaggtcgcttatacctccaaccatacccgatgccactggggggcccaaggaaagcttaccctcactgaggtctccggactcgggtcatgcatagggaaggtgcctcttacccatcaacatctttgcaaccagaccttacccatcaattcctctaaaaaccatcagtatctgctcccctcaaaccatagctggtgggcctgcagcactggcctcaccccctgcctctccacctcagtttttaatcagtctaaagacttctgtgtccaggtccagctgatcccccgcatctattaccattctgaagaaaccttgttacaagcctatgacaaatcaccccccaggtttaaaagagagcctgcctcacttaccctagctgtcttcctggggttagggattgcggcaggtataggtactggctcaaccgccctaattaaagggcccatagacctccagcaaggcctaaccagcctccaaatcgccattgacgctgacctccgggcccttcaggactcaatcagcaagctagaggactcactgacttccctatctgaggtagtactccaaaataggagaggccttgacttactattccttaaagaaggaggcctctgcgcggccctaaaagaagagtgctgtttttatgtagaccactcaggtgcagtacgagactccatgaaaaaacttaaagaaagactagataaaagacagttagagcgccagaaaaaccaaaactggtatgaagggtggttcaataactccccttggtttactaccctactatcaaccatcgctgggcccctattgctcctccttttgttactcactcttgggccctgcatcatcaataaattaatccaattcatcaatgataggataagtgcagtcaaaattttagtccttagacagaaatatcagaccctagataacgaggaaaacctttaa

SEQ ID NO: 54 - DNA, 1530 bp, Artificial Sequence
  FUG Envelope
atggttccgcaggttcttttgtttgtactccttctgggtttttcgttgtgtttcgggaagttccccatttacacgataccagacgaacttggtccctggagccctattgacatacaccatctcagctgtccaaataacctggttgtggaggatgaaggatgtaccaacctgtccgagttctcctacatggaactcaaagtgggatacatctcagccatcaaagtgaacgggttcacttgcacaggtgttgtgacagaggcagagacctacaccaactttgttggttatgtcacaaccacattcaagagaaagcatttccgccccaccccagacgcatgtagagccgcgtataactggaagatggccggtgaccccagatatgaagagtccctacacaatccataccccgactaccactggcttcgaactgtaagaaccaccaaagagtccctcattatcatatccccaagtgtgacagatttggacccatatgacaaatcccttcactcaagggtcttccctggcggaaagtgctcaggaataacggtgtcctctacctactgctcaactaaccatgattacaccatttggatgcccgagaatccgagaccaaggacaccttgtgacatttttaccaatagcagagggaagagagcatccaacgggaacaagacttgcggctttgtggatgaaagaggcctgtataagtctctaaaaggagcatgcaggctcaagttatgtggagttcttggacttagacttatggatggaacatgggtcgcgatgcaaacatcagatgagaccaaatggtgccctccagatcagttggtgaatttgcacgactttcgctcagacgagatcgagcatctcgttgtggaggagttagttaagaaaagagaggaatgtctggatgcattagagtccatcatgaccaccaagtcagtaagtttcagacgtctcagtcacctgagaaaacttgtcccagggtttggaaaagcatataccatattcaacaaaaccttgatggaggctgatgctcactacaagtcagtccggacctggaatgagatcatcccctcaaaagggtgtttgaaagttggaggaaggtgccatcctcatgtgaacggggtgtttttcaatggtataatattagggcctgacgaccatgtcctaatcccagagatgcaatcatccctcctccagcaacatatggagttgttggaatcttcagttatccccctgatgcaccccctggcagacccttctacagttttcaaagaaggtgatgaggctgaggattttgttgaagttcacctccccgatgtgtacaaacagatctcaggggttgacctgggtctcccgaactggggaaagtatgtattgatgactgcaggggccatgattggcctggtgttgatattttccctaatgacatggtgcagagttggtatccatctttgcattaaattaaagcacaccaagaaaagacagatttatacagacatagagatgaaccgacttggaaagtaa

SEQ ID NO: 55 - DNA, 1497 bp, Artificial Sequence
  LCMV Envelope
atgggtcagattgtgacaatgtttgaggctctgcctcacatcatcgatgaggtgatcaacattgtcattattgtgcttatcgtgatcacgggtatcaaggctgtctacaattttgccacctgtgggatattcgcattgatcagtttcctacttctggctggcaggtcctgtggcatgtacggtcttaagggacccgacatttacaaaggagtttaccaatttaagtcagtggagtttgatatgtcacatctgaacctgaccatgcccaacgcatgttcagccaacaactcccaccattacatcagtatggggacttctggactagaattgaccttcaccaatgattccatcatcagtcacaacttttgcaatctgacctctgccttcaacaaaaagacctttgaccacacactcatgagtatagtttcgagcctacacctcagtatcagagggaactccaactataaggcagtatcctgcgacttcaacaatggcataaccatccaatacaacttgacattctcagatcgacaaagtgctcagagccagtgtagaaccttcagaggtagagtcctagatatgtttagaactgccttcggggggaaatacatgaggagtggctggggctggacaggctcagatggcaagaccacctggtgtagccagacgagttaccaatacctgattatacaaaatagaacctgggaaaaccactgcacatatgcaggtccttttgggatgtccaggattctcctttcccaagagaagactaagttcttcactaggagactagcgggcacattcacctggactttgtcagactcttcaggggtggagaatccaggtggttattgcctgaccaaatggatgattcttgctgcagagcttaagtgtttcgggaacacagcagttgcgaaatgcaatgtaaatcatgatgccgaattctgtgacatgctgcgactaattgactacaacaaggctgctttgagtaagttcaaagaggacgtagaatctgccttgcacttattcaaaacaacagtgaattctttgatttcagatcaactactgatgaggaaccacttgagagatctgatgggggtgccatattgcaattactcaaagttttggtacctagaacatgcaaagaccggcgaaactagtgtccccaagtgctggcttgtcaccaatggttcttacttaaatgagacccacttcagtgatcaaatcgaacaggaagccgataacatgattacagagatgttgaggaaggattacataaagaggcaggggagtacccccctagcattgatggaccttctgatgttttccacatctgcatatctagtcagcatcttcctgcaccttgtcaaaataccaacacacaggcacataaaaggtggctcatgtccaaagccacaccgattaaccaacaaaggaatttgtagttgtggtgcatttaaggtgcctggtgtaaaaaccgtctggaaaagacgctga

SEQ ID NO: 56 - DNA, 1692 bp, Artificial Sequence
  FPV Envelope
atgaacactcaaatcctggttttcgcccttgtggcagtcatccccacaaatgcagacaaaatttgtcttggacatcatgctgtatcaaatggcaccaaagtaaacacactcactgagagaggagtagaagttgtcaatgcaacggaaacagtggagcggacaaacatccccaaaatttgctcaaaagggaaaagaaccactgatcttggccaatgcggactgttagggaccattaccggaccacctcaatgcgaccaatttctagaattttcagctgatctaataatcgagagacgagaaggaaatgatgtttgttacccggggaagtttgttaatgaagaggcattgcgacaaatcctcagaggatcaggtgggattgacaaagaaacaatgggattcacatatagtggaataaggaccaacggaacaactagtgcatgtagaagatcagggtcttcattctatgcagaaatggagtggctcctgtcaaatacagacaatgctgctttcccacaaatgacaaaatcatacaaaaacacaaggagagaatcagctctgatagtctggggaatccaccattcaggatcaaccaccgaacagaccaaactatatgggagtggaaataaactgataacagtcgggagttccaaatatcatcaatcttttgtgccgagtccaggaacacgaccgcagataaatggccagtccggacggattgattttcattggttgatcttggatcccaatgatacagttacttttagtttcaatggggctttcatagctccaaatcgtgccagcttcttgaggggaaagtccatggggatccagagcgatgtgcaggttgatgccaattgcgaaggggaatgctaccacagtggagggactataacaagcagattgccttttcaaaacatcaatagcagagcagttggcaaatgcccaagatatgtaaaacaggaaagtttattattggcaactgggatgaagaacgttcccgaaccttccaaaaaaaggaaaaaaagaggcctgtttggcgctatagcagggtttattgaaaatggttgggaaggtctggtcgacgggtggtacggtttcaggcatcagaatgcacaaggagaaggaactgcagcagactacaaaagcacccaatcggcaattgatcagataaccggaaagttaaatagactcattgagaaaaccaaccagcaatttgagctaatagataatgaattcactgaggtggaaaagcagattggcaatttaattaactggaccaaagactccatcacagaagtatggtcttacaatgctgaacttcttgtggcaatggaaaaccagcacactattgatttggctgattcagagatgaacaagctgtatgagcgagtgaggaaacaattaagggaaaatgctgaagaggatggcactggttgctttgaaatttttcataaatgtgacgatgattgtatggctagtataaggaacaatacttatgatcacagcaaatacagagaagaagcgatgcaaaatagaatacaaattgacccagtcaaattgagtagtggctacaaagatgtgatactttggtttagcttcggggcatcatgctttttgcttcttgccattgcaatgggccttgttttcatatgtgtgaagaacggaaacatgcggtgcactatttgtatataa

SEQ ID NO: 57 - DNA, 1266 bp, Artificial Sequence
  RRV Envelope
agtgtaacagagcactttaatgtgtataaggctactagaccatacctagcacattgcgccgattgcggggacgggtacttctgctatagcccagttgctatcgaggagatccgagatgaggcgtctgatggcatgcttaagatccaagtctccgcccaaataggtctggacaaggcaggcacccacgcccacacgaagctccgatatatggctggtcatgatgttcaggaatctaagagagattccttgagggtgtacacgtccgcagcgtgctccatacatgggacgatgggacacttcatcgtcgcacactgtccaccaggcgactacctcaaggtttcgttcgaggacgcagattcgcacgtgaaggcatgtaaggtccaatacaagcacaatccattgccggtgggtagagagaagttcgtggttagaccacactttggcgtagagctgccatgcacctcataccagctgacaacggctcccaccgacgaggagattgacatgcatacaccgccagatataccggatcgcaccctgctatcacagacggcgggcaacgtcaaaataacagcaggcggcaggactatcaggtacaactgtacctgcggccgtgacaacgtaggcactaccagtactgacaagaccatcaacacatgcaagattgaccaatgccatgctgccgtcaccagccatgacaaatggcaatttacctctccatttgttcccagggctgatcagacagctaggaaaggcaaggtacacgttccgttccctctgactaacgtcacctgccgagtgccgttggctcgagcgccggatgccacctatggtaagaaggaggtgaccctgagattacacccagatcatccgacgctcttctcctataggagtttaggagccgaaccgcacccgtacgaggaatgggttgacaagttctctgagcgcatcatcccagtgacggaagaagggattgagtaccagtggggcaacaacccgccggtctgcctgtgggcgcaactgacgaccgagggcaaaccccatggctggccacatgaaatcattcagtactattatggactataccccgccgccactattgccgcagtatccggggcgagtctgatggccctcctaactctggcggccacatgctgcatgctggccaccgcgaggagaaagtgcctaacaccgtacgccctgacgccaggagcggtggtaccgttgacactggggctgctttgctgcgcaccgagggcgaatgca

SEQ ID NO: 58 - DNA, 1938 bp, Artificial Sequence
  MLV 10A1 Envelope
atggaaggtccagcgttctcaaaaccccttaaagataagattaacccgtggaagtccttaatggtcatgggggtctatttaagagtagggatggcagagagcccccatcaggtctttaatgtaacctggagagtcaccaacctgatgactgggcgtaccgccaatgccacctcccttttaggaactgtacaagatgccttcccaagattatattttgatctatgtgatctggtcggagaagagtgggacccttcagaccaggaaccatatgtcgggtatggctgcaaataccccggagggagaaagcggacccggacttttgacttttacgtgtgccctgggcataccgtaaaatcggggtgtggggggccaagagagggctactgtggtgaatggggttgtgaaaccaccggacaggcttactggaagcccacatcatcatgggacctaatctcccttaagcgcggtaacaccccctgggacacgggatgctccaaaatggcttgtggcccctgctacgacctctccaaagtatccaattccttccaaggggctactcgagggggcagatgcaaccctctagtcctagaattcactgatgcaggaaaaaaggctaattgggacgggcccaaatcgtggggactgagactgtaccggacaggaacagatcctattaccatgttctccctgacccgccaggtcctcaatatagggccccgcatccccattgggcctaatcccgtgatcactggtcaactacccccctcccgacccgtgcagatcaggctccccaggcctcctcagcctcctcctacaggcgcagcctctatagtccctgagactgccccaccttctcaacaacctgggacgggagacaggctgctaaacctggtagaaggagcctatcaggcgcttaacctcaccaatcccgacaagacccaagaatgttggctgtgcttagtgtcgggacctccttattacgaaggagtagcggtcgtgggcacttataccaatcattctaccgccccggccagctgtacggccacttcccaacataagcttaccctatctgaagtgacaggacagggcctatgcatgggagcactacctaaaactcaccaggccttatgtaacaccacccaaagtgccggctcaggatcctactaccttgcagcacccgctggaacaatgtgggcttgtagcactggattgactccctgcttgtccaccacgatgctcaatctaaccacagactattgtgtattagttgagctctggcccagaataatttaccactcccccgattatatgtatggtcagcttgaacagcgtaccaaatataagagggagccagtatcgttgaccctggcccttctgctaggaggattaaccatgggagggattgcagctggaatagggacggggaccactgccctaatcaaaacccagcagtttgagcagcttcacgccgctatccagacagacctcaacgaagtcgaaaaatcaattaccaacctagaaaagtcactgacctcgttgtctgaagtagtcctacagaaccgaagaggcctagatttgctcttcctaaaagagggaggtctctgcgcagccctaaaagaagaatgttgtttttatgcagaccacacgggactagtgagagacagcatggccaaactaagggaaaggcttaatcagagacaaaaactatttgagtcaggccaaggttggttcgaagggcagtttaatagatccccctggtttaccaccttaatctccaccatcatgggacctctaatagtactcttactgatcttactctttggaccctgcattctcaatcgattggtccaatttgttaaagacaggatctcagtggtccaggctctggttttgactcaacaatatcaccagctaaaacctatagagtacgagccatga

SEQ ID NO: 59 - DNA, 2030 bp, Artificial Sequence
  EboV Envelope
atgggtgttacaggaatattgcagttacctcgtgatcgattcaagaggacatcattctttctttgggtaattatccttttccaaagaacattttccatcccacttggagtcatccacaatagcacattacaggttagtgatgtcgacaaactggtttgccgtgacaaactgtcatccacaaatcaattgagatcagttggactgaatctcgaagggaatggagtggcaactgacgtgccatctgcaactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccacaaagagggtgctttcttcctgtatgaccgacttgcttccacagttatctaccgaggaacgactttcgctgaaggtgtcgttgcatttctgatactgccccaagctaagaaggacttcttcagctcacaccccttgagagagccggtcaatgcaacggaggacccgtctagtggctactattctaccacaattagatatcaagctaccggttttggaaccaatgagacagagtatttgttcgaggttgacaatttgacctacgtccaacttgaatcaagattcacaccacagtttctgctccagctgaatgagacaatatatacaagtgggaaaaggagcaataccacgggaaaactaatttggaaggtcaaccccgaaattgatacaacaatcggggagtgggccttctgggaaactaaaaaaacctcactagaaaaattcgcagtgaagagttgtctttcacagctgtatcaaacagagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctgacaacccttgccacaatctccacgagtcctcaaccccccacaaccaaaccaggtccggacaacagcacccacaatacacccgtgtataaacttgacatctctgaggcaactcaagttgaacaacatcaccgcagaacagacaacgacagcacagcctccgacactccccccgccacgaccgcagccggacccctaaaagcagagaacaccaacacgagcaagggtaccgacctcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggaggagagctcgaagagaagcaattgtcaatgctcaacccaaatgcaaccctaatttacattactggactactcaggatgaaggtgctgcaatcggactggcctggataccatatttcgggccagcagccgagggaatttacatagaggggctgatgcacaatcaagatggtttaatctgtgggttgagacagctggccaacgagacgactcaagctcttcaactgttcctgagagccacaaccgagctacgcaccttttcaatcctcaaccgtaaggcaattgatttcttgctgcagcgatggggcggcacatgccacattttgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggcgttataattgcagttatcgctttattctgtatatgcaaatttgtcttttag

SEQ ID NO: 60 - DNA, 218 bp, Artificial Sequence
  Thyroxin binding globulin promoter(TBG)
ctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgag

SEQ ID NO: 61 - DNA, 523 bp, Artificial Sequence
  DNA fragment containing prothrombin enhancer and human alpha-1 anti-trypsin promoter
gcgagaacttgtgcctccccgtgttcctgctctttgtccctctgtcctacttagactaatatttgccttgggtactgcaaacaggaaatgggggagggacaggagtagggcggagggtagcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 62 - DNA, 558 bp, Artificial Sequence
  DNA fragment containing prothrombin enhancer, human alpha-1 anti-trypsin promoter, and five HNF1 binding sites
gttaatcattaacgttaatcattaacgttaatcattaacgttaatcattaacgttaatcattaacatcgatgcgagaacttgtgcctccccgtgttcctgctctttgtccctctgtcctacttagactaatatttgccttgggtactgcaaacaggaaatgggggagggacaggagtagggcggagggtaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 63 - DNA, 589 bp, Artificial Sequence
  DNA fragment containing prothrombin enhancer, human alpha-1 anti-trypsin promoter, and three HNF1/HNF4 binding sites
gttaatcattaacgcttgtactttggtacagttaatcattaacgcttgtactttggtacagttaatcattaacgcttgtactttggtacaatcgatgcgagaacttgtgcctccccgtgttcctgctctttgtccctctgtcctacttagactaatatttgccttgggtactgcaaacaggaaatgggggagggacaggagtagggcggagggtagcccggggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 64 - DNA, 24 bp, Artificial Sequence
  hPAH FAM TaqMan Probe
tcgtgaaagctcatggacagtggc

SEQ ID NO: 65 - DNA, 22 bp, Artificial Sequence
  PAH TaqMan Forward Primer
agatcttgaggcatgacattgg

SEQ ID NO: 66 - DNA, 22 bp, Artificial Sequence
  PAH TaqMan Reverse Primer
gtccagctcttgaatggttctt

SEQ ID NO: 67 - DNA, 20 bp, Artificial Sequence
  Actin FAM Probe
agcgggaaatcgtgcgtgac

SEQ ID NO: 68 - DNA, 21 bp, Artificial Sequence
  Actin Forward Primer
ggacctgactgactacctcat

SEQ ID NO: 69 - DNA, 22 bp, Artificial Sequence
  Actin Reverse Primer
cgtagcacagcttctccttaat

SEQ ID NO: 70 - DNA, 1359 bp, Artificial Sequence
  Codon-optimized PAH (OPT3)
atgtctaccgccgtgctggaaaatcctggcctgggcagaaagctgagcgacttcggccaagagacaagctacatcgaggacaactgcaaccagaacggcgccatcagcctgatcttcagcctgaaagaagaagtgggcgccctggccaaggtgctgagactgttcgaagagaacgacgtgaacctgacacacatcgagagcagacccagcagactgaagaaggacgagtacgagttcttcacccacctggacaagcggagcctgcctgctctgaccaacatcatcaagatcctgcggcacgacatcggcgccacagtgcacgaactgagccgggacaagaaaaaggacaccgtgccatggttccccagaaccatccaagagctggacagattcgccaaccagatcctgagctatggcgccgagctggacgctgatcaccctggctttaaggaccccgtgtaccgggccagaagaaagcagtttgccgatatcgcctacaactaccggcacggccagcctattcctcgggtcgagtacatggaagaggaaaagaaaacctggggcaccgtgttcaagaccctgaagtccctgtacaagacccacgcctgctacgagtacaaccacatcttcccactgctcgagaagtactgcggcttccacgaggacaatatccctcagctcgaggacgtgtcccagttcctgcagacctgcaccggctttagactgaggcctgttgccggactgctgagcagcagagattttctcggcggcctggccttcagagtgttccactgtacccagtacatcagacacggcagcaagcccatgtacacccctgagcctgatatctgccacgagctgctgggacatgtgcccctgttcagcgatagaagcttcgcccagttcagccaagagatcggactggcttctctgggagcccctgacgagtacattgagaagctggccaccatctactggttcaccgtggagttcggcctgtgcaagcagggcgatagcatcaaggcttatggcgctggcctgctgtctagctttggcgagctgcagtactgtctgagcgagaagcctaagctgctgcccctggaactggaaaagaccgccatccagaactacaccgtgaccgagttccagcctctgtactacgtggccgagagcttcaacgacgccaaagaaaaagtgcggaacttcgccgccaccattcctcggcctttcagcgtcagatacgacccctacacacagcggatcgaggtgctggacaacacacagcagctgaaaattctggccgacagcatcaacagcgagatcggcatcctgtgcagcgccctgcagaaaatcaagtga

SEQ ID NO: 71 - DNA, 1359 bp, Artificial Sequence
  Codon-optimized PAH (OPT2/3)
atgagtacggctgtgctcgagaatccaggtttgggccgaaagctgtctgattttggacaggagacatcttatattgaagacaactgcaaccagaatggtgcgatatcccttattttttctctgaaagaagaagtaggtgcgctggcaaaggtcttgcggctgtttgaagagaacgatgttaatcttactcatattgagtccagaccatcacggctgaaaaaagacgagtacgaattttttactcacttggacaaacgaagcttgccggctcttactaatatcattaagatcctccggcatgacataggggcgacagtgcatgagctttcaagggataaaaagaaagataccgtcccctggtttccaaggaccatacaagaactcgaccgattcgcgaaccagatcctttcatatggtgctgagttggatgctgaccaccccggcttcaaagacccggtctaccgagcgcggcggaaacaatttgctgacatcgcatacaattacaggcatggccagccaattcctagagtagaatacatggaagaagagaaaaaaacctggggtaccgtcttcaagacgctgaaatcattgtataaaactcatgcatgttacgaatataaccatatttttccgttgctcgagaaatattgcgggttccacgaagataacatcccacaactcgaggatgtatctcagttcctccagacctgtacggggtttcgacttaggcctgttgccggactgctgagcagcagagattttctcggcggcctggccttcagagtgttccactgtacccagtacatcagacacggcagcaagcccatgtacacccctgagcctgatatctgccacgagctgctgggacatgtgcccctgttcagcgatagaagcttcgcccagttcagccaagagatcggactggcttctctgggagcccctgacgagtacattgagaagctggccaccatctactggttcaccgtggagttcggcctgtgcaagcagggcgatagcatcaaggcttatggcgctggcctgctgtctagctttggcgagctgcagtactgtctgagcgagaagcctaagctgctgcccctggaactggaaaagaccgccatccagaactacaccgtgaccgagttccagcctctgtactacgtggccgagagcttcaacgacgccaaagaaaaagtgcggaacttcgccgccaccattcctcggcctttcagcgtcagatacgacccctacacacagcggatcgaggtgctggacaacacacagcagctgaaaattctggccgacagcatcaacagcgagatcggcatcctgtgcagcgccctgcagaaaatcaagtga

SEQ ID NO: 72 - DNA, 1359 bp, Artificial Sequence
  Codon-optimized PAH (OPT3/2)
atgtctaccgccgtgctggaaaatcctggcctgggcagaaagctgagcgacttcggccaagagacaagctacatcgaggacaactgcaaccagaacggcgccatcagcctgatcttcagcctgaaagaagaagtgggcgccctggccaaggtgctgagactgttcgaagagaacgacgtgaacctgacacacatcgagagcagacccagcagactgaagaaggacgagtacgagttcttcacccacctggacaagcggagcctgcctgctctgaccaacatcatcaagatcctgcggcacgacatcggcgccacagtgcacgaactgagccgggacaagaaaaaggacaccgtgccatggttccccagaaccatccaagagctggacagattcgccaaccagatcctgagctatggcgccgagctggacgctgatcaccctggctttaaggaccccgtgtaccgggccagaagaaagcagtttgccgatatcgcctacaactaccggcacggccagcctattcctcgggtcgagtacatggaagaggaaaagaaaacctggggcaccgtgttcaagaccctgaagtccctgtacaagacccacgcctgctacgagtacaaccacatcttcccactgctcgagaagtactgcggcttccacgaggacaatatccctcagctcgaggacgtgtcccagttcctgcagacctgcaccggctttagactgaggcctgtcgcgggtttgctcagttctcgagacttcctgggtggattggcgtttcgggtattccattgcacgcagtatatccgacacggaagtaagccaatgtacacgccagaacccgatatctgtcacgaattgcttggacacgttcctctgttttctgatcgatcattcgctcagttttcacaggaaatcggcctggcatctttgggagcgccggatgaatatattgagaagctcgctacaatttactggttcacggtagaatttgggttgtgcaagcagggtgatagtattaaagcatacggtgcgggattgctgtcctcattcggggagcttcagtattgcctgtccgagaaacccaagctgttgccgttggaattggaaaaaaccgctatccaaaattacacagtaacggagttccaacctttgtactacgtagccgagtcatttaacgatgcaaaggagaaggtcagaaattttgctgcgacgatacccagaccgttctcagtaaggtacgatccttacactcagaggattgaagtcctggataatacgcaacagctcaagatcctggcagactccataaattctgaaatcggcatcttgtgttcagcactgcaaaagataaaataa

SEQ ID NO: 73 - DNA, 15 bp, Artificial Sequence
  DNA Fragment of OPT3
agaaccatccaagag

SEQ ID NO: 74 - DNA, 19 bp, Artificial Sequence
  DNA Fragment of OPT3
tattcctcgggtcgagtac

SEQ ID NO: 75 - DNA, 16 bp, Artificial Sequence
  DNA Fragment of OPT3
agagatcggactggct

SEQ ID NO: 76 - DNA, 15 bp, Artificial Sequence
  DNA Fragment of OPT3
tcctcggcctttcag

SEQ ID NO: 77 - DNA, 529 bp, Artificial Sequence
  DNA fragment containing prothrombin enhancer, human alpha-1, anti-trypsin promoter, and one HNF1/HNF4 binding site
gttaatcattaacgcttgtactttggtacaatcgatgcgagaacttgtgcctccccgtgttcctgctctttgtccctctgtcctacttagactaatatttgccttgggtactgcaaacaggaaatgggggagggacaggagtagggcggagggtagcccggggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 78 - DNA, 615 bp, Artificial Sequence
  Prothrombin enhancer-hAAT promoter-Minute Virus of Mouse intron
gcgagaacttgtgcctccccgtgttcctgctctttgtccctctgtcctacttagactaatatttgccttgggtactgcaaacaggaaatgggggagggacaggagtagggcggagggtagcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaataagaggtaagggtttaagggatggttggttggtggggtattaatgtttaattacctggagcacctgcctgaaatcactttttttcaggttgg

SEQ ID NO: 79 - DNA, 334 bp, Artificial Sequence
  hAAT promoter-Transthyretin enhancer-Minute Virus of Mouse intron
gggggaggctgctggtgaatattaaccaaggtcaccccagttatcggaggagcaaacaggggctaagtccaccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcaaagaggtaagggtttaagggatggttggttggtggggtattaatgtttaattacctggagcacctgcctgaaatcactttttttcaggttgg

SEQ ID NO: 80 - DNA, 92 bp, Artificial Sequence
  Minute virus of Mouse intron
aagaggtaagggtttaagggatggttggttggtggggtattaatgtttaattacctggagcacctgcctgaaatcactttttttcaggttgg

SEQ ID NO: 81 - DNA, 171 bp, Artificial Sequence
  Transthyretin enhancer
ccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatca

SEQ ID NO: 82 - DNA, 71 bp, Artificial Sequence
  hAAT promoter
gggggaggctgctggtgaatattaaccaaggtcaccccagttatcggaggagcaaacaggggctaagtcca

SEQ ID NO: 83 - DNA, 1654 bp, Artificial Sequence
  PAH optimized version 3-PAH 3UTR
atgtctaccgccgtgctggaaaatcctggcctgggcagaaagctgagcgacttcggccaagagacaagctacatcgaggacaactgcaaccagaacggcgccatcagcctgatcttcagcctgaaagaagaagtgggcgccctggccaaggtgctgagactgttcgaagagaacgacgtgaacctgacacacatcgagagcagacccagcagactgaagaaggacgagtacgagttcttcacccacctggacaagcggagcctgcctgctctgaccaacatcatcaagatcctgcggcacgacatcggcgccacagtgcacgaactgagccgggacaagaaaaaggacaccgtgccatggttccccagaaccatccaagagctggacagattcgccaaccagatcctgagctatggcgccgagctggacgctgatcaccctggctttaaggaccccgtgtaccgggccagaagaaagcagtttgccgatatcgcctacaactaccggcacggccagcctattcctcgggtcgagtacatggaagaggaaaagaaaacctggggcaccgtgttcaagaccctgaagtccctgtacaagacccacgcctgctacgagtacaaccacatcttcccactgctcgagaagtactgcggcttccacgaggacaatatccctcagctcgaggacgtgtcccagttcctgcagacctgcaccggctttagactgaggcctgttgccggactgctgagcagcagagattttctcggcggcctggccttcagagtgttccactgtacccagtacatcagacacggcagcaagcccatgtacacccctgagcctgatatctgccacgagctgctgggacatgtgcccctgttcagcgatagaagcttcgcccagttcagccaagagatcggactggcttctctgggagcccctgacgagtacattgagaagctggccaccatctactggttcaccgtggagttcggcctgtgcaagcagggcgatagcatcaaggcttatggcgctggcctgctgtctagctttggcgagctgcagtactgtctgagcgagaagcctaagctgctgcccctggaactggaaaagaccgccatccagaactacaccgtgaccgagttccagcctctgtactacgtggccgagagcttcaacgacgccaaagaaaaagtgcggaacttcgccgccaccattcctcggcctttcagcgtcagatacgacccctacacacagcggatcgaggtgctggacaacacacagcagctgaaaattctggccgacagcatcaacagcgagatcggcatcctgtgcagcgccctgcagaaaatcaagtgagtcgacagccatggacagaatgtggtctgtcagctgtgaatctgttgatggagatccaactatttctttcatcagaaaaagtccgaaaagcaaaccttaatttgaaataacagccttaaatcctttacaagatggagaaacaacaaataagtcaaaataatctgaaatgacaggatatgagtacatactcaagagcataatggtaaatcttttggggtcatctttgatttagagatgataatcccatactctcaattgagttaaatcagtaatctgtcgcatttcatcaagatta

SEQ ID NO: 84 - DNA, 1995 bp, Artificial Sequence
  PAH optimized version 3-Albumin 3UTR
atgtctaccgccgtgctggaaaatcctggcctgggcagaaagctgagcgacttcggccaagagacaagctacatcgaggacaactgcaaccagaacggcgccatcagcctgatcttcagcctgaaagaagaagtgggcgccctggccaaggtgctgagactgttcgaagagaacgacgtgaacctgacacacatcgagagcagacccagcagactgaagaaggacgagtacgagttcttcacccacctggacaagcggagcctgcctgctctgaccaacatcatcaagatcctgcggcacgacatcggcgccacagtgcacgaactgagccgggacaagaaaaaggacaccgtgccatggttccccagaaccatccaagagctggacagattcgccaaccagatcctgagctatggcgccgagctggacgctgatcaccctggctttaaggaccccgtgtaccgggccagaagaaagcagtttgccgatatcgcctacaactaccggcacggccagcctattcctcgggtcgagtacatggaagaggaaaagaaaacctggggcaccgtgttcaagaccctgaagtccctgtacaagacccacgcctgctacgagtacaaccacatcttcccactgctcgagaagtactgcggcttccacgaggacaatatccctcagctcgaggacgtgtcccagttcctgcagacctgcaccggctttagactgaggcctgttgccggactgctgagcagcagagattttctcggcggcctggccttcagagtgttccactgtacccagtacatcagacacggcagcaagcccatgtacacccctgagcctgatatctgccacgagctgctgggacatgtgcccctgttcagcgatagaagcttcgcccagttcagccaagagatcggactggcttctctgggagcccctgacgagtacattgagaagctggccaccatctactggttcaccgtggagttcggcctgtgcaagcagggcgatagcatcaaggcttatggcgctggcctgctgtctagctttggcgagctgcagtactgtctgagcgagaagcctaagctgctgcccctggaactggaaaagaccgccatccagaactacaccgtgaccgagttccagcctctgtactacgtggccgagagcttcaacgacgccaaagaaaaagtgcggaacttcgccgccaccattcctcggcctttcagcgtcagatacgacccctacacacagcggatcgaggtgctggacaacacacagcagctgaaaattctggccgacagcatcaacagcgagatcggcatcctgtgcagcgccctgcagaaaatcaagtgagtcgacattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaagttatggattataaacattcaaaataatattttgacattatgataattctgaataaaagaacaaaaaccatggtataggtaaggaatataaaacatggcttttaccttagaaaaaacaattctaaaattcatatggaatcaaaaaagagcctgca

SEQ ID NO: 85 - DNA, 255 bp, Artificial Sequence
  PAH 3UTR
agccatggacagaatgtggtctgtcagctgtgaatctgttgatggagatccaactatttctttcatcagaaaaagtccgaaaagcaaaccttaatttgaaataacagccttaaatcctttacaagatggagaaacaacaaataagtcaaaataatctgaaatgacaggatatgagtacatactcaagagcataatggtaaatcttttggggtcatctttgatttagagatgataatcccatactctcaattgagt

SEQ ID NO: 86 - DNA, 630 bp, Artificial Sequence
  Albumin 3UTR
attcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaagttatggattataaacattcaaaataatattttgacattatgataattctgaataaaagaacaaaaaccatggtataggtaaggaatataaaacatggcttttaccttagaaaaaacaattctaaaattcatatggaatcaaaaaagagcctgca

SEQ ID NO: 87 - DNA, 395 bp, Artificial Sequence
  WPREs (WPRE without X-protein sequence)
aatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggtacc